(19) United States
(12) Patent Application Publication
(10) Pub. No.: US 2002/0128301 A1
(43) Pub. Date: Sep. 12, 2002

(54) Non-nucleoside reverse transcriptase inhibitors

(75) Inventors: Xiao-Xiong Zhou, Huddinge (SE); Nils-Gunnar Johansson, Enhorna (SE); Horst Wahling, Skarholmen (SE); Christian Sund, Varby (SE); Lourdes Salvador, Norrkoping (SE); Stefan Lindstrom, Uppsala (SE); Hans Wallberg, Huddinge (SE); Christer Sahlberg, Hagersten (SE)

Correspondence Address: BIRCH STEWART KOLASCH & BIRCH, PO BOX 747, FALLS CHURCH, VA, 22040-0747, US

(73) Assignee: Medivir AB, Huddinge (SE)

(21) Appl. No.: 09/927,254

(22) Filed: Aug. 10, 2001

Related U.S. Application Data
(63) Continuation-in-part of application No. 09/249,317, filed on Feb. 12, 1999.
(63) Continuation-in-part of application No. PCT/SE99/1403, filed on Aug. 18, 1999.

(30) Foreign Application Priority Data
Feb. 13, 1998 (SE) 9800452-6
Apr. 3, 1998 (SE) 9801216-4
Feb. 16, 1998 (SE) 9800469-0
Oct. 7, 1998 (SE) 9803438-2
Aug. 13, 1998 (ZA) 98/7267
Aug. 14, 1998 (SE) PCT/SE98/01467
Feb. 15, 1999 (SE) PCT/SE99/00194

(51) Int. Cl. A61K 31/4172; A61K 31/404; A61K 31/401; A61K 31/277; A61K 31/223

(52) U.S. Cl. 514/400; 514/419; 514/423; 514/521; 514/534; 514/551; 514/550; 548/339.1; 548/496; 548/552; 558/269; 560/40; 560/155

(57) ABSTRACT

Non-nucleoside reverse transcriptase inhibitors of formula (P-1) wherein: Ar1 is an unsaturated, optionally substituted, mono or bicyclic ring structure comprising 0 to 3 hetero atoms selected from S, O and N; Ar2 is an aromatic, optionally substituted, monocyclic ring structure comprising at least one nitrogen hetero atom and zero to two further hetero atoms selected from S, O and N; R4 and R5 are independently H or C₃-C₈ cycloalkyl, C₁-C₆ alkyl, C₂-C₆ alkenyl, C₂-C₆ alkynyl, C₁-C₅ alkoxy, C₁-C₄ alkanoyloxy, C₁-C₄ alkylthio, amino, carboxy, carbamoyl, cyano, halo, hydroxy, aminomethyl, hydroxymethyl, carboxymethyl, or halo substituted C₁-C₆ alkyl mercapto, nitro; or R4 and RS join to form a 3-6 membered, optionally substituted ring structure; R6 is 0 or S; Rx is the residue of a natural or unnatural amino acid; and L* is a linker moiety which is ether-, carbonate- or ester-bound to the adjacent oxygen and ester linked to Rx; and pharmaceutically acceptable salts thereof are anti-HIV agents with favourable pharmacokinetic properties.

### TECHNICAL FIELD

This invention relates to non-nucleoside reverse transcriptase inhibitors active against HIV-1 and having improved pharmacokinetic properties. The invention further relates to the synthesis of such compounds and their use in antiviral methods and compositions.

### BACKGROUND OF THE INVENTION

Non nucleoside reverse transcriptase inhibitors (NNRTI) bind to an allosteric site on reverse transcriptase and represent an important development in the arsenal of drugs against HIV, particularly HIV-1. International patent application WO 93/03022, discloses thiourea NNRTI, later denoted PETT (phenyl ethyl thiazolyl thiourea) compounds in J Med Chem 39 6 1329-1335 (1995) and J Med Chem 39 21 4261-4274 (1996). International patent application no WO95/06034 discloses urea isosteres of PETT NNRTIs. PCT/SE99/00053 published after the priority and international filing dates of the present application discloses particularly active urea NNTRI compounds with the formula:

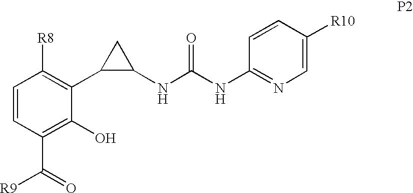

wherein

R8 is halo;

R9 is C₁-C₃ alkyl;

R10 is halo, especially bromo or cyano.

The contents of each of the above documents are specifically incorporated by reference.

Although the urea and thiourea NNRTI disclosed in the above documents are extremely active against reverse transcriptase, especially that of HIV-1, the nature of the HIV virus with its extreme lack of replicative fidelity and consequent tendency to rapid resistance development prompts a demand for further antiretroviral agents. Additionally, modern HIV therapy regimes, denoted HAART, Highly Active Anti Retroviral Therapy, administer antivirals as combinations of three or more antivirals of various classes, which combinations are administered for prolonged periods, if not for life. HAART requires the patient to follow a complicated dosing schedule with sometimes dozens of tablets per day taken at various times of the day in some cases before and in other cases after the ingestion of food. There is thus a need for antiretroviral preparations allowing greater flexibility in dosing to facilitate patient compliance.

To pursuit of such flexibility of dosing, the abovementioned PCT/SE99/00053 proposes certain prodrugs of the compounds of formula P2 above building on the phenolic hydroxy group. These prodrugs comprise aminobenzoyl or pyridyl esters of this phenolic hydroxy, wherein the amino group may be optionally substituted with an amino acid peptidically bound thereto.

### BRIEF DESCRIPTION OF THE INVENTION

We have now discovered that a particular class of prodrug provides enhanced pharmacokinetic performance in respect of NNRTIs of the PETT class.

Accordingly a first aspect of the invention provides compounds of the formula

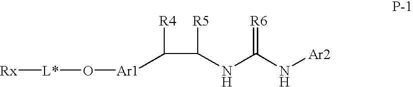

wherein:

Ar1 is an unsaturated, optionally substituted, mono or bicyclic ring structure comprising 0 to 3 hetero atoms selected from S, O and N;

Ar2 is an aromatic, optionally substituted, monocyclic ring structure comprising at least one nitrogen hetero atom and zero to two further hetero atoms selected from S, O and N;

R4 and R5 are independently H, a substituent selected from C₃-C₈ cycloalkyl, C₁-C₆ alkyl, C₂-C₆ alkenyl, C₂-C₈ alkynyl, C₁-C₅ alkoxy, C₁-C₄ alkanoyloxy, C₁-C₄ alkylthio, amino, carboxy, carbamoyl, cyano, halo, hydroxy, aminomethyl, hydroxymethyl, carboxymethyl, or halo substituted C₁-C₆ alkyl mercapto, nitro, or R4 and R5 join to form a 3-6 membered, optionally substituted ring structure;

R6 is O or S;

L* is a linker moiety which is ether-, carbonate- or ester-bound to the adjacent oxygen and ester linked to Rx;

Rx is the residue of a natural or unnatural amino acid;

and pharmaceutically acceptable salts thereof.

Favoured Ar1 groups include phenyl, pyridyl, napthyl, indanyl, indolyl, quinolyl, isoquinolyl, benzopyridyl, benzoimidazol, benzothiazolyl, benzopyrazinyl, benzotriazolyl, benzopyrimidyl, benzopyridazinyl, purinyl, pyrazinyl, pyrrolyl pyrazolyl, pyrimidinyl, pyridazinyl, triazolyl, cyclohexenyl and the like, especially pyrid-2-yl, pyrid-3-yl and phenyl. The —O—L*—Rx moiety is conveniently located at the 2 or 5 position of ring Ar1 relative to the linkage to the rest of the molecule.

Favoured Ar2 groups include pyridyl, pyrimidinyl, pyrrolyl, pyrazinyl, pyridazinyl, triazolyl, thiazolyl, thiadiazolyl and the like, especially pyridyl. Preferably Ar2 comprises a nitrogen atom at the 2-position relative to the linkage to the (thio)urea. The optional substituent to Ar2, if present, is preferably located para to the linkage to the (thio)urea.

R6 is preferably oxygen, thus defining a urea PETT derivative. R4 and R5 as a ring structure can comprise optionally substituted cyclopropyl, cyclobutyl, cyclopentyl or the like, preferably cis-cyclopropyl. Alternatively R4 and R5 are conveniently H.

The optional substituents described herein conveniently comprise up to three substituents selected from C₁-C₆ alkyl, C₁-C₆ alkoxy, C₂-C₆ alkenyl, C₂-C₈ alkynyl, C₂-C₈ alkenoxy, C₁-C₆ alkoxy C₁-C₆ alkyl, C₁-C₆ alkanoyl, haloC₁-C₆ alkyl, C₁-C₄ alkanoyloxy, C₁-C₄ alkylthio, amino, carboxy, carbamoyl, cyano, halo, hydroxy, aminomethyl, carboxymethyl, hydroxymethyl, nitro, aryl, substituted (as herein defined) aryl, or —SO₂D or —C(═O)D where D is C₁-C₆ alkyl, aryl, substituted (as herein defined) aryl or amino;

The optional substituent to Ar2 preferably comprises halo, especially bromo or cyano. The optional substituent(s) to Ar1 comprise one or two members independently selected from the group consisting H, fluoro, bromo, chloro, hydroxy, methoxy, ethoxy, amino, dimethylamino, acetyl, propionyl, butyryl and the like. Where Ar2 is a six membered ring, the optional substituents are conveniently located at positions 2 and/or 6 relative to the linkage to the rest of the molecule.

C₁-Cₙ alkyl includes such groups as methyl, ethyl, n-propyl, isopropyl, n-butyl, s-butyl, t-butyl, n-pentyl, n-hexyl, 3-methyl pentyl and the like. The term halo refers to chloro, bromo, fluoro and iodo. C₁-Cₙ alkoxy refers to groups such as methoxy, ethoxy, propoxy, t-butoxy and the like. C₂-Cₙ alkenyl,refers to groups such as vinyl, 1-propen-2-yl, 1-buten-4-yl, 1-penten-5-yl, 1-buten-1-yl and the like. C₁-Cₙ alkylthio includes methylthio, ethylthio, t-butylthio and the like. C₁-Cₙ alkanoyloxy includes acetoxy, propionoxy, formyloxy, butyryloxy and the like. C₂-Cₙ alkenoxy includes ethenyloxy, propenyloxy, isobutoxyethenyl and the like. HaloC₁-Cₙ alkyl includes alkyls as defined herein substituetd 1 to 3 times bya halogen including trifluormethyl, 2-dichloroethyl, 3,3-difluoropropyl and the like. The term amine includes goups such as NH₂, NHMe, N(Me)₂ which may optionally be substituted with halogen, C₁-C₇ acyloxy, C₁-C₆ alkyl, C₁-C₆ alkoxy, nitro, carboxy, carbamoyl, carbamoyloxy cyano, methylsulphonylamino and the like. Carboxy, carboxymethyl and carbamoyl include the corresponding pharmaceutically acceptable C₁-C₆ alkyl and aryl esters.

Particularly preferred compounds of the present invention have the formula

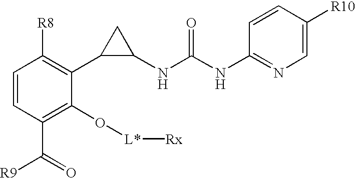

wherein

R8 is halo, especially halo;

R9 is C₁-C₃ alkyl;

R10 is halo or cyano;

and L*Rx is as defined above.

A preferred subset of compounds within Formula P2, particularly with regard to pharmacokinetics, has R10 as cyano. A further favoured subset of compounds within Formula P2, particularly with regard to ease of forming prodrugs, comprise compounds wherein R10 is bromo. Preferably R8 is chloro and more preferably fluoro. Suitable R9 groups include methyl, isopropyl, n-propyl and preferably ethyl.

As depicted in Formula P2, the cyclopropyl ring is in the cis configuration, allowing two enantiomers, 1S,2S and 1R,2R

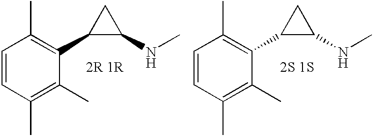

Each of these enantiomers are potent antiretrovirals, although the different enantiomers can display subtle differences in physiological properties. For instance the 1S,2S and 1R,2R enantiomers can show a different pattern of metabolism within the P450 system. The 1S,2S enantiomer of compounds wherein Rp10 is cyano is particularly preferred as it appears unique in being able to avoid key components of the P450 system. Other retroviral agents such as the HIV protease inhibitor ritonavir interact extensively with the P450 system, leading to an array of undesirable physiological responses including extensive alteration of the metabolism of other co-administered drugs. This is of particular concern with pharmaceuticals administered for a chronic infection where patients can expect to take a number of pharmaceuticals for years, if not decades.

Preferred NNRTI mother compounds for preparing prodrugs in accordance with the invention thus include:

(1S,2S)-N-[cis-2-(6-fluoro, 2-hydroxy, 3-propionylphenyl)-cyclopropyl]-N′-(5-cyanopyrid-2-yl)-urea,

(1S,2S)-N-[cis-2-(6-fluoro, 2-hydroxy, 3-butyrylphenyl)-cyclopropyl]-N′-(5-cyanopyrid-2-yl)-urea,

(1S,2S)-N-[cis-2-(6-fluoro, 2-hydroxy, 3-acetylphenyl)-cyclopropyl]-N′-(5-cyanopyrid-2-yl)-urea,

(1S,2S)-N-[cis-2-(6-fluoro, 2-hydroxy, 3-propionylphenyl)-cyclopropyl]-N′-(5-bromopyrid-2-yl)-urea,

(1S,2S)-N-[cis-2-(6-fluoro, 2-hydroxy, 3-butyrylphenyl)-cyclopropyl]-N′-(5-bromopyrid-2-yl)-urea,

(1S,2S)-N-[cis-2-(6-fluoro, 2-hydroxy, 3-acetylphenyl)-cyclopropyl]-N′-(5-bromopyrid-2-yl)-urea,

and the corresponding 1R,2R enantiomers.

As the compounds of formula P1 and in particular P2 generally include an electron withdrawing group on the phenol/heteroarylol ring to which the prodrug moiety is attached, it is generally preferred to avoid a simple ester linkage for L*. Accordingly L* preferably comprises an ether or carbonate linkage to the phenolic (or other heteroarylic) hydroxy of the mother compound. A particularly convenient ether linkage has the formula:

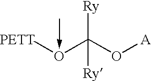

Where PETT is the dehydroxy residue of the NNRTI, the arrowed oxygen is the phenolic (or other heterocyclic) hydroxy, Ry and Ry′ are independently H S or C₁-C₃ alkyl and A is the esterified amino acid residue Rx. Alternatively, A can comprise an intermediate linking moiety to which Rx is esterified. Examples of such an intermediate linking moiety include a straight or branched chain hydroxycarboxylic acid derivative, such as 3-hydroxypropionate, 4-hydroxybutyrate and in particular L-lactic acid, wherein the carboxy function is esterified to the non-arrowed oxygen above and the hydroxy function is esterified to the amino acid. The intemediate linking moiety may comprise an arylic or heteroaryl component bearing an (alkylenyl)carboxy function amenable for esterification with the amino acid of Rx. The form and preparation of linking groups suitable for use in the compounds of the invention is extensively described in our copending application no PCT/SE99/00194, the contents of which are incorporated by reference.

Alternatively A as an intermediate linking moiety can comprise a carbonate linkage to the alkyloxy group depicted above, in which case A further comprises a branched or straight chain hydroxycarboxylic acid derivative amenable to esterification with the amino acid of Rx. Similarly a carbonate linkage bearing A moiety may comprise an arylic or heteroaryl component bearing an (alkylenyl)carboxy function amenable for esterification with the amino acid of Rx.

Thus a convenient group of prodrugs within the scope of the invention include those of the formula P3:

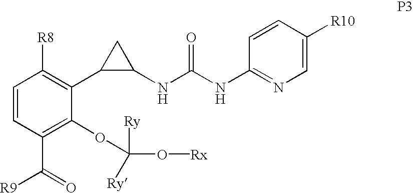

wherein

R8, R9, R10 are as described above;

Ry and Ry′ are independently H or C₁-C₃ alkyl;

Rx is the ester residue of a natural or unnatural amino acid;

and pharmaceutically acceptable salts thereof.

Typically both of Ry and Ry′ are H. Rx is preferably the ester residue of an aliphatic amino acid such as alanine, tertiary leucine or leucine, preferably isoleucine and especially valine, Preferably Rx is derived from an L-amino acid.

Preferred compounds within Formula P3 thus include;

(1S,2S)-N-{cis-2-[6-fluoro-2-(L-valyloxymethyloxy)-3-propionylphenyl]cyclopropyl}-N′-[2-(5-cyanopyridyl)]urea,

(1S,2S)-N-{cis-2-[6-fluoro-2-(L-isoleucyloxymethyloxy)-3-propionylphenyl]cyclopropyl}-N′-[2-(5-cyanopyridyl)]urea,

(1R,2R)-N-{cis-2-[6-fluoro-2-(L-valyloxymethyloxy)-3-propionylphenyl]cyclopropyl}-N′-[2-(5-cyanopyridyl)]urea,

(1R,2R)-N-{cis-2-[6-fluoro-2-(L-isoleucyloxymethyloxy)-3-propionylphenyl]cyclopropyl}-N′-[2-(5-cyanopyridyl)]urea,

(1S,2S)-N-{cis-2-[6-fluoro-2-(L-valyloxymethyloxy)-3-propionylphenyl]cyclopropyl}-N′-[2-(5-bromopyridyl)]urea,

(1S,2S)-N-{cis-2-[6-fluoro-2-(L-isoleucyloxymethyloxy)-3-propionylphenyl]cyclopropyl}-N′-[2-(5-bromopyridyl)]urea,

(1R,2R)-N-{cis-2-[6-fluoro-2-(L-valyloxymethyloxy)-3-propionylphenyl]cyclopropyl}-N′-[2-(5-bromopyridyl)]urea,

(1R,2R)-N-{cis-2-[6-fluoro-2-(L-isoleucyloxymethyloxy)-3-propionylphenyl]cyclopropyl}-N′-[2-(5-bromopyridyl)]urea,

and pharmaceutically acceptable salts thereof.

An alternative preferred group of prodrugs of the invention have the Formula P4:

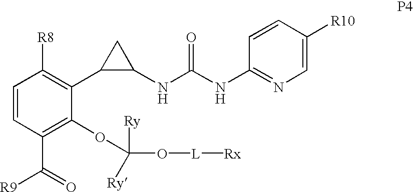

where R8, R9, R10, Ry and Ry′ are as defined above. L and R₂ define a linker group and residue of an amino acid, such as those depicted in copending application PCT/SE99/00194.

Typically both of Ry and Ry′ are H. Rx is preferably the ester residue of an aliphatic amino acid such as alanine, tertiary leucine or leucine, preferably isoleucine and especially valine, Preferably Rx is derived from an L-amino acid.

Favoured compounds within formula P4 include those of the formula P5:

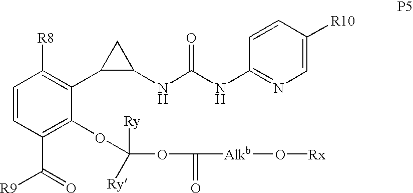

where R8 R9, R10, Ry, Ry′ and Rx are as defined above and Alk^{b} is C₁-C₆ optionally branched, optionally monounsaturated alkyl.

Favoured compounds within Formula P5 thus include:

(1S,2S)-N-{cis-2-[6-fluoro-2-(2,2-dimethyl-3-(L-valyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(2-methyl-3-(L-valyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(3-(L-valyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(2,2-dimethyl-3-(L-valyloxy)-propionyl-oxymethyloxy)-3-propionylphenyl]-cyclopropyl}-N′-[2-(5-bromopyridyl)]urea,

(1S,2S)-N-{cis-2-[6-fluoro-2-(2-methyl-3-(L-valyloxy)-propionyl-oxymethyloxy)-3-propionylphenyl]-cyclopropyl}-N′-[2-(5-bromopyridyl)]urea,

(1S,2S)-N-{cis-2-[6-fluoro-2-(3-(L-valyloxy)-propionyl-oxymethyloxy)-3-propionylphenyl]-cyclopropyl}-N′-[2-(5-bromopyridyl)]urea,

(1S,2S)-N-{cis-2-[6-fluoro-2-(2,2-dimethyl-3-(L-isoleucyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(2-methyl-3-(L-isoleucyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(3-(L-isoleucyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(2,2-dimethyl-3-(L-isoleucyloxy)-propionyl-oxymethyloxy)-3-propionylphenyl]-cyclopropyl}-N′-[2-(5-bromopyridyl)]urea,

(1S,2S)-N-{cis-2-[6-fluoro-2-(2-methyl-3-(L-isoleucyloxy)-propionyl-oxymethyloxy)-3-propionylphenyl]-cyclopropyl}-N′-[2-(5-bromopyridyl)]urea,

(1S,2S)-N-{cis-2-[6-fluoro-2-(3-(L-isoleucyloxy)-propionyl-oxymethyloxy)-3-propionylphenyl]-cyclopropyl}-N′-[2-(5-bromopyridyl)]urea,

(1S,2S)-N-{cis-2-[6-fluoro-2-(4-(L-valyloxy)-butyryl-oxymethyloxy)-3-propionylphenyl]-cyclopropyl}-N′-[2-(5-cyanopyridyl)]urea,

(1S,2S)-N-{cis-2-[6-fluoro-2-(4-(L-isoleucyloxy)-butyryl-oxymethyloxy)-3-propionylphenyl]-cyclopropyl}-N′-[2-(5-cyanopyridyl)]urea,

(1S,2S)-N-{cis-2-[6-fluoro-2-(4-(L-valyloxy)-butyryl-oxymethyloxy)-3-propionylphenyl]-cyclopropyl}-N′-[2-(5-bromopyridyl)]urea,

(1S,2S)-N-{cis-2-[6-fluoro-2-(4-(L-isoleucyloxy)-butyryl-oxymethyloxy)-3-propionylphenyl]-cyclopropyl}-N′-[2-(5-bromopyridyl)]urea,

(1S,2S)-N-{cis-2-[6-fluoro-2-(2-(L-valyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(2-(L-isoleucyloxy)-propionyl-oxymethyloxy)-3-propionylphenyl]-cyclopropyl}-N′-[2-(5-cyanopyridyl)]urea,

(1S,2S)-N-{cis-2-[6-fluoro-2-(2-(L-valyloxy)-propionyl-oxymethyloxy)-3-propionylphenyl]-cyclopropyl}-N′-[2-(5-bromopyridyl)]urea,

(1S,2S)-N-{cis-2-[6-fluoro-2-(2-(L-isoleucyloxy)-propionyl-oxymethyloxy)-3-propionylphenyl]-cyclopropyl}-N′-[2-(5-bromopyridyl)]urea,

and the corresponding (1R,2R) enantiomers thereof.

One variant of a branched Alk^{b} in Formula P5 can be substituted with hydroxy which in turn is esterified with a further Rx, thus defining a compound of the Formula P6:

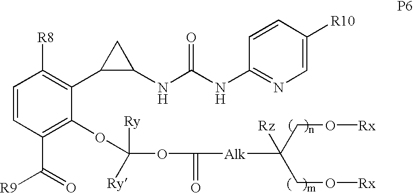

where R8, R9, R10, Alk, Ry, Ry′, and Rx are as defined above, Rz is H or C₁-C₃ alkyl ( ) represents a methylene group and m and n are independently 0, 1 or 2.

Particularly favoured values for Alk, m and n include: methylene:1:1 and absent: 1:0 respectively.

A further favoured group of compounds has the Formula P7:

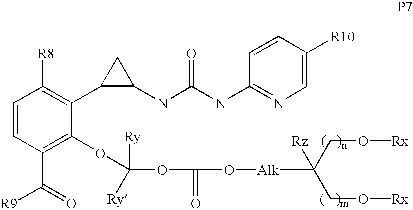

where R8, R9, R10, Alk, Ry, Ry′, m, n Rz and Rx are as defined above or wherein the —( )ₘ—O—R₂ arm is absent.

Typically both of Ry and Ry′ are H. Preferably Rz is H. Rx is preferably the ester residue of an aliphatic amino acid such as alanine, tertiary leucine or leucine, preferably isoleucine and especially valine, Preferably Rx is derived from an L-amino acid.

Particularly favoured values for Alk, m and n include:absent:1:1, thus defining a glycerol derivative.

Where the —( )ₘ—O—R₂ arm is absent in formula P7, the compounds have the structure P7′:

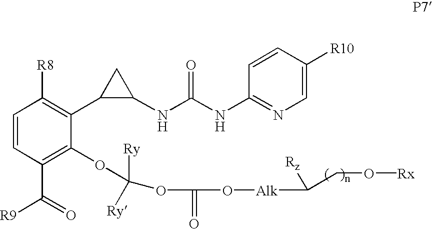

Convenient values for Alk and n include absent:1 with Ry, Ry and Rz′ as H.

Favoured compounds within Formula P-7 thus include

(1S,2S)-N-[cis-2-(6-fluoro-2-(1,3-bis-L-valyloxy-2-(oxycarbonylmethoxy)propyl)-3-propionylphenyl)cyclopropyl]-N′-[2-(5-cyanopyridyl)]urea,

(1S,2S)-N-[cis-2-(6-fluoro-2-(1,3-bis-L-isoleucyloxy-2-(oxycarbonylmethoxy)propyl)-3-propionylphenyl)cyclopropyl]-N′-[2-(5-cyanopyridyl)]urea,

(1S,2S)-N-[cis-2-(6-fluoro-2-(1,3-bis-L-valyloxy-2-(oxycarbonylmethoxy)propyl)-3-propionylphenyl)cyclopropyl]-N′-[2-(5-bromopyridyl)]urea,

(1S,2S)-N-[cis-2-(6-fluoro-2-(1,3-bis-L-isoleucyloxy-2-(oxycarbonylmethoxy)propyl)-3-propionylphenyl)cyclopropyl]-N′-[2-(5-bromopyridyl)]urea,

(1S,2S)-N-{cis-2-[6-fluoro-2-(2-(L-valyloxy)-ethoxycarbonyloxymethyloxy)-3-propionylphenyl)]cyclopropyl}-N′-[2-(5-cyanopyridyl)]urea,

(1S,2S)-N-{cis-2-[6-fluoro-2-(2-(L-isoleucyloxy)-ethoxycarbonyloxymethyloxy)-3-propionylphenyl)]cyclopropyl}-N′-[2-(5-cyanopyridyl)]urea,

(1S,2S)-N-{cis-2-[6-fluoro-2-(2-(L-valyloxy)-ethoxycarbonyloxymethyloxy)-3-propionylphenyl)]cyclopropyl}-N′-[2-(5-bromopyridyl)]urea,

(1S,2S)-N-{cis-2-[6-fluoro-2-(2-(L-isoleucyloxy)-ethoxycarbonyloxymethyloxy)-3-propionylphenyl)]cyclopropyl}-N′-[2-(5-bromocyanopyridyl)]urea,

and the corresponding R,R enantiomers.

A further favoured group of compounds omit the alkyleneoxy group immediately adjacent the phenolic (or other heteroaryl) hydroxy function of the compound of formula P1 or P2. An example of such compounds has the formula P8:

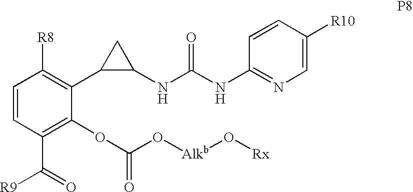

where R8, R9, R10, Rx, and Alk^{b} are as defined above.

Currently favoured values for Alk^{b} include methylene, ethylene, 1,1-dimethylethylene, 2,2-isopropylene, butylene and, in the case of said -ORx substitution described below, glycerol.

Favoured compounds within formula P-8 thus include:

(1S,2S)-N-[cis-2-(6-fluoro-2-(L-valyloxymethoxycarbonyloxy)-3-propionylphenyl)cyclopropyl]-N′-[2-(5-cyanopyridyl)]urea,

(1S,2S)-N-[cis-2-(6-fluoro-2-(L-isoleucyloxymethoxycarbonyloxy)-3-propionylphenyl)cyclopropyl]-N′-[2-(5-cyanopyridyl)]urea, (1S,2S)-N-[cis-2-(6-fluoro-2-(L-valyloxymethoxycarbonyloxy)-3-propionylphenyl)cyclopropyl]-N′-[2-(5-bromopyridyl)]urea,

(1S,2S)-N-[cis-2-(6-fluoro-2-(L-isoleucyloxymethoxycarbonyloxy)-3-propionylphenyl)cyclopropyl]-N′-[2-(5-bromopyridyl)]urea,

(1S,2S)-N-[cis-2-(6-fluoro-2-(2-(L-valyloxy)ethoxycarbonyloxy)-3-propionylphenyl)cyclopropyl]-N′-[2-(5-cyanopyridyl)]urea,

(1S,2S)-N-[cis-2-(6-fluoro-2-(2-(L-isoleucyloxy)ethoxycarbonyloxy)-3-propionylphenyl)cyclopropyl]-N′-[2-(5-cyanopyridyl)]urea,

(1S,2S)-N-[cis-2-(6-fluoro-2-(2-(L-valyloxy)ethoxycarbonyloxy)-3-propionylphenyl)cyclopropyl]-N′-[2-(5-bromopyridyl)]urea,

(1S,2S)-N-[cis-2-(6-fluoro-2-(2-L-isoleucyloxy)ethoxycarbonyloxy)-3-propionylphenyl)cyclopropyl]-N′-[2-(5-bromopyridyl)]urea,

(1S,2S)-N-[cis-2-(6-fluoro-2-(3-(L-valyloxy)propoxycarbonyloxy)-3-propionylphenyl)cyclopropyl]-N′-[2-(5-cyanopyridyl)]urea,

(1S,2S)-N-[cis-2-(6-fluoro-2-(3-(L-isoleucyloxy)propoxycarbonyloxy)-3-propionylphenyl)cyclopropyl]-N′-[2-(5-cyanopyridyl)]urea,

(1S,2S)-N-[cis-2-(6-fluoro-2-(3-(L-valyloxy)propoxycarbonyloxy)-3-propionylphenyl)cyclopropyl]-N′-[2-(5-bromopyridyl)]urea,

(1S,2S)-N-[cis-2-(6-fluoro-2-(3-(L-isoleucyloxy)propoxycarbonyloxy)-3-propionylphenyl)cyclopropyl]-N′-[2-(5-bromopyridyl)]urea,

(1S,2S)-N-[cis-2-(6-fluoro-2-(4-(L-valyloxy)butoxycarbonyloxy)-3-propionylphenyl)cyclopropyl]-N′-[2-(5-cyanopyridyl)]urea,

(1S,2S)-N-[cis-2-(6-fluoro-2-(4-(L-isoleucyloxy)butoxycarbonyloxy)-3-propionylphenyl)cyclopropyl]-N′-[2-(5-cyanopyridyl)]urea,

(1S,2S)-N-[cis-2-(6-fluoro-2-(4-(L-valyloxy)butoxycarbonyloxy)-3-propionylphenyl)cyclopropyl]-N′-[2-(5-bromopyridyl)]urea,

(1S,2S)-N-[cis-2-(6-fluoro-2-(4-(L-isoleucyloxy)butoxycarbonyloxy)-3-propionylphenyl)cyclopropyl]-N′-[2-(5-bromopyridyl)]urea,

and the corresponding R, R enantiomers

As with Formula P5/P6 and P7/P7′, Alk^{b} in formula P8 can comprise an additional —O—Rz substitution to define a compound of the formula P8′

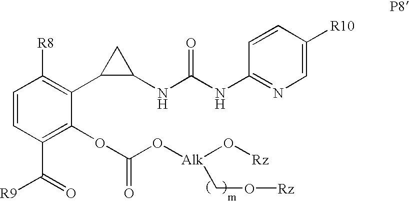

where each of the variables is as defined above.

The invention further provides pharmaceutical compositions comprising the compounds of formula P1 and pharmaceutically acceptable carriers or diluents therefor. Additional aspects of the invention provide methods for the inhibition of HIV comprising administering a compound of the formula I to a subject afflicted with HIV. The invention also extends to the use of the compounds of formula I in therapy, such as in the preparation of a medicament for the treatment of HIV infections.

In treating conditions caused by HIV, the compounds of formula I are preferably administered in an amount to achieve a plasma level of the compounds of Formula P1 of around 10 to 1000 nM and more preferably 100 to 500 nM. This corresponds to a dosage rate, depending on the bioavailability of the formulation, of the order 0.01 to 10 mg/kg/day, preferably 0.1 to 2 mg/kg/day. A typical dosage rate for a normal adult will be around 0.05 to 5 g per day, preferably 0.1 to 2 g such as 500-750 mg, in one to four dosage units per day.

In keeping with the usual practice with HIV inhibitors it is advantageous to co-administer one to three additional antivirals to provide synergistic responses and to ensure complementary resistance patterns. Such additional antivirals may include AZT, ddI, ddC, D4T, 3TC, abacavir, adefovir, adefovir dipivoxil, bis-POC-PMPA, foscarnet, hydroxyurea, Hoechst-Bayer HBY 097, efavirenz, trovirdine, nevirapine, delaviridine, PFA, H2G (tamociclovir), ABT 606 (valtamociclovir stearate) DMP-450, loviride, ritonavir, saquinavir, indinavir, amprenavir (Vertex VX 478), nelfinavir and the like, typically at molar ratios reflecting their respective activities and bioavailabilities. Generally such ratio will be of the order of 25:1 to 1:25, relative to the compound of formula I.

Compounds of the invention are typically prepared by alkylation of the corresponding mother compounds of Formula P1 or especially P2, which are prepared by conventional means, such as the methodology described in WO95/06034 or PCT/SE99/00053.

In particular, the preparation of compounds of formula P-3 or P-4 generally proceeds by alkylation using conventional coupling conditions of a compound of the formula P-2 with the corresponding intermediate:

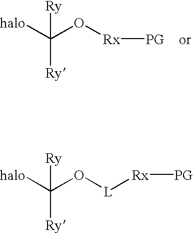

where Ry, Ry′ and L are as defined above and Rx—PG is Rx as defined, but N-protected with a conventional N-protecting group. Preferably the halogen activating group is iodo, which is in turn prepared by iodination of the corresponding chloro analogue. Typical coupling conditions include treatment with a base in an organic solvent such as THF prior to addition of the activated (halogenated) intermediate followed by conventional deprotection of the Rx N-protecting group.

Compounds of formula P8 are generally prepared by esterification of a compound of the formula P2 with an intermediate of the formula:

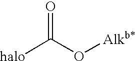

where Alk^{b}* is a functionalised Alk^{b} as described above, for example chloromethyl chloroformate, in an organic solvent, followed by iodination of the terminal chloro with NaI (or other activation of the functionalising group) and esterification/alkylation with an N-protected Rx.

After coupling of the activated L*-(N-protected)-Rx group to the mother NNRTI (or the two step coupling of the activated L* moiety followed by coupling of the N-protected Rx) the N-protecting group on the amino acid Rx is removed by conventional techniques such as palladium catalysed reduction or treatment with an organic acid such as trifluoroacetic acid.

Typical intermediates for a one step coupling thus comprise

iodomethyloxy-N-CBz-valyl,

iodomethyloxy-N-Boc-valyl,

iodomethyloxy-N-Fmoc-valyl

iodomethyloxy-N-CBz-isoleucyl,

iodomethyloxy-N-Boc-isoleucyl,

iodomethyloxy-N-Fmoc-isoleucyl,

iodomethyloxy-N-CBz-tertiaryleucyl,

iodomethyloxy-N-Boc-tertiaryleucyl,

iodomethyloxy-N-Fmoc-tertiaryleucyl,

and corresponding derivatives bearing other N-protecting groups.

Further typical intermediates for a one step coupling include

2,2-dimethyl-3-(N-Boc-L-valyloxy)propionic acid iodomethyl ester

3,3-bis(N-CBz-L-valyloxymethyl)-propionic acid iodomethyl ester,

2-(N-CBz-L-valyloxy)ethoxycarbonyloxymethyl iodide

Iodomethyl 1,3-bis(N-benzyloxycarbonyl-L-valyloxy)-2-propyl carbonate,

Iodomethyl 2-methyl-2-(N-benzyloxycarbonyl-L-valyloxymethyl)propionate,

Iodomethyl 2-(N-benzyloxycarbonyl-L-valyloxy)-DL-propionate.

Iodomethyl 2-(N-benzyloxycarbonyl-L-valyloxy)isobutyrate.

Iodomethyl 2-(N-benzyloxycarbonyl-L-valyloxy)-3-methyl-(S)-(+)-butyrate.

Iodomethyl 2-O-(N-benzyloxycarbonyl-L-valyloxy)-2-phenyl-DL-acetate

Iodomethyl 4-(N-benzyloxycarbonyl-L-valyloxy) benzoate.

Iodomethyl 5-(N-CBz-L-valyloxy)-2,2-dimethylvalerate

2-(N-CBz-L-valyloxy)-ethyl iodomethyl carbonate

4-(N-CBz-L-valyloxy) butyric acid iodomethyl ester

Iodomethyl-3-(N-benzyloxycarbonyl-L-valyloxy)-benzoate

Iodomethyl-3-(N-benzyloxycarbonyl-L-valyloxy)-propionate

1,3-bis(N-tert-butoxycarbonyl-L-valyloxy)-2-propyl 1-iodoethyl carbonate

3-(N-benzyloxycarbonyl-L-valyloxy)-2,2-dimethylpropyl iodomethyl carbonate

Iodomethyl 3,4-di-(N-CBZ-L-valyloxy)hydrocinnamate

3-(N-CBZ-L-valyloxy)phenyl iodomethyl carbonate

Iodomethyl 2-(N-CBZ-L-valyloxy)phenylacetate

Iodomethyl 4-(N-CBZ-L-valyloxyxy)phenylacetate

Iodomethyl 4-(2-N-benzyloxycarbonyl-L-valyloxyethyl)benzoate

Iodomethyl 4-(N-benzyloxycarbonyl-L-valyloxy)cyclohexanoate.

Iodomethyl 2-(N-benzyloxycarbonyl-L-valyloxymethyl )-2-ethyl butyrate

2-(N-(iodomethoxycarbonyl)-amino)-2-methyl-1-(N-benzyloxycarbonyl-L-valyloxy)-propane

1-(2-N-CBz-L-valyloxyethyl)-6-oxo-1,6-dihydro-pyridine-3-carboxylic acid iodomethyl ester

Iodomethyl 5-[(N-benzyloxycarbonyl-L-valyloxy)methyl]-2-furoate

Iodomethyl 4-(2-N-benzyloxycarbonyl-L-valyloxyethoxy)-benzoic acid

2,2-dimethyl-3-(N-Boc-L-isoleucyloxy)propionic acid iodomethyl ester

3,3-bis(N-CBz-L-isoleucyloxymethyl)-propionic acid iodomethyl ester,

2-(N-CBz-L-isoleucyloxy)ethoxycarbonyloxymethyl iodide

Iodomethyl 1,3-bis(N-benzyloxycarbonyl-L-isoleucyloxy)-2-propyl carbonate,

Iodomethyl 2-methyl-2-(N-benzyloxycarbonyl-L-isoleucyloxymethyl)propionate,

Iodomethyl 2-(N-benzyloxycarbonyl-L-isoleucyloxy)-DL-propionate.

Iodomethyl 2-(N-benzyloxycarbonyl-L-isoleucyloxy)isobutyrate.

Iodomethyl 2-(N-benzyloxycarbonyl-L-isoleucyloxy)-3-methyl-(S)-(+)-butyrate.

Iodomethyl 2-(N-benzyloxycarbonyl-L-isoleucyloxy)-2-phenyl-DL-acetate

Iodomethyl 4-(N-benzyloxycarbonyl-L-isoleucyloxy) benzoate.

Iodomethyl 5-(N-CBz-L-isoleucyloxy)-2,2-dimethylvalerate

2-(N-CBz-L-isoleucyloxy)-ethyl iodomethyl carbonate

4-(N-CBz-L-isoleucyloxy) butyric acid iodomethyl ester

Iodomethyl-3-(N-benzyloxycarbonyl-L-isoleuclyloxy)-benzoate

Iodomethyl-3-(N-benzyloxycarbonyl-L-isoleucyloxy)-propionate

1,3-bis(N-tert-butoxycarbonyl-L-isoleucyloxy)-2-propyl 1-iodoethyl carbonate

3-(N-benzyloxycarbonyl-L-isoleucyloxy)-2,2-dimethylpropyl iodomethyl carbonate

Iodomethyl 3,4-di-(N-CBz-L-isoleucyloxy)hydrocinnamate

3-(N-CBz-L-isoleucyloxy)phenyl iodomethyl carbonate

Iodomethyl 2-(N-CBz-L-isoleucyloxy)phenylacetate

Iodomethyl 4-(N-CBz-L-isoleucyloxy)phenylacetate

Iodomethyl 4-(2-N-benzyloxycarbonyl-L-isoleucyloxyethyl)benzoate

Iodomethyl 4-(N-benzyloxycarbonyl-L-isoleucyloxy)cyclohexanoate,

Iodomethyl 2-(N-benzyloxycarbonyl-L-isoleucyloxymethyl)-2-ethyl butyrate,

2-(N-(iodomethoxycarbonyl)-amino)-2-methyl-1-(N-benzyloxycarbonyl-L-isoleucyloxy)-propane,

1-(2-N-CBz-L-isoleucyloxyethyl)-6-oxo-1,6-dihydro-pyridine-3-carboxylic acid iodomethyl ester

iodomethyl 5-[(N-benzyloxycarbonyl-L-isoleucyloxy)methyl]-2-furoate

iodomethyl 4-(2-N-benzyloxycarbonyl-L-isoleucyloxyethoxy)-benzoic acid

and the corresponding chloromethyl analogues.

Although the above list describes Boc and CBz N-protecting groups on the amino acid, it will be appreciated that other N-protecting groups are also available.

Copending international application no PCT/SE99/00194 comprehensively describes the preparation of analogous intermediates.

The term “N-protecting group” or “N-protected” as used herein refers to those groups intended to protect the N-terminus of an amino acid or peptide or to protect an amino group against undesirable reactions during synthetic procedures. Commonly used N-protecting groups are disclosed in Greene, “Protective Groups in Organic Synthesis” (John Wiley & Sons, New York, 1981), which is hereby incorporated by reference. N-protecting groups include acyl groups such as formyl, acetyl, propionyl, pivaloyl, t-butylacetyl, 2-chloroacetyl, 2-bromoacetyl, trifluoracetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl, and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butoxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like; alkyl gropus such as benzyl, triphenylmethyl, benzyloxymethyl and the like; and silyl groups such as trimethylsilyl and the like. Favoured N-protecting groups include formyl, acetyl, benzoyl, pivaloyl, t-butylacetyl, phenylsulfonyl, benzyl, t-butoxycarbonyl (BOC) and benzyloxycarbonyl (Cbz).

Hydroxy and/or carboxy protecting groups are also extensively reviewed in Greene ibid and include ethers such as methyl, substituted methyl ethers such as methoxymethyl, methylthiomethyl, benzyloxymethyl, t-butoxymethyl, 2-methoxyethoxymethyl and the like, silyl ethers such as trimethylsilyl (TMS), t-butyldimethylsilyl (TBDMS) tribenzylsilyl, triphenylsilyl, t-butyldiphenylsilyl triisopropyl silyl and the like, substituted ethyl ethers such as 1-ethoxymethyl, 1-methyl-1-methoxyethyl, t-butyl, allyl, benzyl, p-methoxybenzyl, dipehenylmethyl, triphenylmethyl and the like, aralkyl groups such as trityl, and pixyl (9-hydroxy-9-phenylxanthene derivatives, especially the chloride). Ester hydroxy protecting groups include esters such as formate, benzylformate, chloroacetate, methoxyacetate, phenoxyacetate, pivaloate, adamantoate, mesitoate, benzoate and the like. Carbonate hydroxy protecting groups include methyl vinyl, allyl, cinnamyl, benzyl and the like.

The compounds of the invention can form salts which form an additional aspect of the invention. Appropriate pharmaceutically acceptable salts of the compounds of Formula I include salts of organic acids, especially carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-napthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. The compounds of the invention I may in some cases be isolated as the hydrate.

While it is possible for the active agent to be administered alone, it is preferable to present it as part of a pharmaceutical formulation. Such a formulation will comprise the above defined active agent together with one or more acceptable carriers or excipients and optionally other therapeutic ingredients. The carrier(s) must be acceptable in the sense of being compatible with the other ingredients of the formulation and not deleterious to the recipient.

The formulations include those suitable for rectal, nasal, topical (including buccal and sublingual), vaginal or parenteral (including subcutaneous, intramuscular, intravenous and intradermal) administration, but preferably the formulation is an orally administered formulation. The formulations may conveniently be presented in unit dosage form, e.g. tablets and sustained release capsules, and may be prepared by any methods well known in the art of pharmacy.

Such methods include the step of bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of Formula I or its pharmaceutically acceptable salt in conjunction or association with a pharmaceutically acceptable carrier or vehicle. If the manufacture of pharmaceutical formulations involves intimate mixing of pharmaceutical excipients and the active ingredient in salt form, then it is often preferred to use excipients which are non-basic in nature, i.e. either acidic or neutral. Formulations for oral administration in the present invention may be presented as discrete units such as capsules, cachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion and as a bolus etc.

With regard to compositions for oral administration (e.g. tablets and capsules), the term suitable carrier includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, stearic acid, glycerol stearate, silicone fluid, talc waxes, oils and colloidal silica. Flavouring agents such as peppermint, oil of wintergreen, cherry flavouring or the like can also be used. It may be desirable to add a colouring agent to make the dosage form readily identifiable. Tablets may also be coated by methods well known in the art. A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

### DETAILED DESCRIPTION

Preparation of Intermediates

### EXAMPLE AA-I-1

### 2,3-Bis-(N-CBz-L-valyloxy)-propionic acid

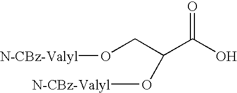

### a) t-Butyl 2,3-bis(N-CBz-L-valyloxy)propionate

To a solution of t-butyl 2,3-dihydroxypropionate (2.43 g, 15 mmole), N-CBz-L-valine (7.54 g, 30 mmole) and DMAP (0.37 g, 3 mmole) in 150 ml dichloromethane was added DCC (7.2 g 35 mmole) and the mixture was stirred for two days at room temperature. The mixture was cooled to about 5° C. and the urethane was filtered. The filtrate was evaporated, ethyl acetate was added and the organic phase washed twice with 5% acetic acid, 5% sodium hydrogen carbonate and water. The organic phase was dried with sodium sulfate filtered and evaporated under reduced pressure. The product was isolated by silica gel column chromatography. Yield: 8.2 g=86%

### b) 2,3-Bis-(N-CBz-L-valyloxy)-propionic acid

To a solution of t-butyl -2,3-bis-(N-CBz-L-valyloxy)-propionate (7.2 g, 11.4 mmole) in dichloromethane (25 ml) was added trifluoroacetic acid (25 ml) and the solution was stirred for five hours at room temperature. The solution was evaporated under reduced pressure and coevaporated two times with toluene. The product was isolated by silica gel column chromatography. Yield: 5.9 g=90% The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

¹H-NMR (DMSO-d6) 0.92 (m, 12H) 2.08 (m, 2H) 3.92-4.17 (m, 2H) 4.30-4.67 (m, 2H) 5.04 (s, 4H) 5.28 (m, 1H) 7.32 (m, 10H) 7.70 (m, 2H)

### EXAMPLE AA-I-2

### (S)-(+)-2-(N-CBz-L-valyloxy)propionic acid

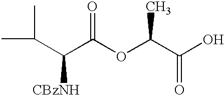

### a) 4-Methoxybenzyl (S)(+)-2-hydroxypropionate

To a stirred solution of (S)(+)2 hydroxypropionic acid (9.0 g, 100 mmole) in 100 ml dry DMF was added potassium tert-butoxide (12.34 g, 110 mmole) and the mixture was stirred for one hour at 25° C. 4-Methoxybenzyl chloride (18.8 g 120 mmole) was added and the mixture was stirred for six hours at 60° C. The mixture was evaporated under reduced pressure and 250 ml ethyl acatate was added. The organic phase was washed four times with water. The organic phase was dried with sodium sulfate and concentrated in vacuo. Yield: 15.6 g=74%

### b) 4-Methoxybenzyl (S)-(+)-2-(N-CBz-L-valyloxy)propionate

To a solution of 4-methoxybenzyl (S)-(+)-2-hydroxypropionate (7.6 g, 36 mmole), N-CBZ-L-valine (10.05 g, 40 mmole) and DMAP (0.98 g, 8 mmole) in 150 ml dichloromethane was added a solution of DCC (8.3 g, 40 mmole) and the mixture was stirred overnight at room temperature. The mixture was cooled to about 5° C. and the urethane was filtered. The filtrate was evaporated and the product was isolated by silica gel column chromatography. Yield: 14.4 g=90%

### c) (S)-(+)-2-(N-CBz-L-valyloxy)propionic acid

To a solution of 4-methoxybenzyl (S)-(+)-2-(N-CBz-L-valyloxy)propionate (14.0 g, 31.5 mmole) in dichloromethane (50 ml) was added trifluoroacetic acid (25 ml) and the solution was stirred for five hours at room temperature. The solution was evaporated under reduced pressure and coevaporated two times with toluene. The product was isolated by silica gel column chromatography. Yield: 9.4 g=92%. The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

¹H-NMR (DMSO-d6) 0.94 (m, 6H) 1.46 (d, 3H) 2.12 (m, 1H) 4.05 (m, 1H) 4.92 (m, 1H) 5.06 (s, 2H) 7.34 (m, 5H) 7.68 (d, 1H)

### EXAMPLE AA-I-3

### 3,3-Bis(N-CBz-L-valyloxymethyl)-propionic acid

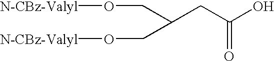

### a) 4,4-bis(N-CBZ-L-valyloxymethyl)-but-1-ene

To a solution of 2-allyl-1,3-propanediol (2.32 g, 20 mmole), N-CBZ-L-valine (10.06 g, 40 mmole) and DMAP (0.488 g, 4 mmole) in 120 ml dichloromethane was added DCC (9.08 g, 44 mmole) in portions and the mixture was stirred overnight at room temperature. The mixture was cooled to 5° C, and the urethane was filtered. The filtrate was evaporated and the product was isolated by silica gel column chromatography. Yield: 9.0 g

### b) 3,3-Bis(N-CBZ-L-valyloxymethyl)-propionic acid

To a cooled solution of 4,4-bis(N-CBZ-L-valyloxymethyl)-but-1-ene (14.6 g, 25 mmole) and tetrabutylammonium bromide (1.3 g, 4 mmole) in 120 ml benzene was added 100 ml water. Under strong stirring potassium permanganate (15.8 g, 100 mmole) was addded in portions and the mixture was stirred for 2 hours between 15° C. and 20° C. A sodium bisulfite aqueous solution was added to the slurry until the mixture was discolored. The mixture was acidified with 2N hydrochloric acid and extracted four times with ethyl acetate. The organic phase was washed two times with water, dried with sodium sulfate and evaporated under reduced pressure. The product was isolated by silica gel column chromatography. Yield: 7.5 g. The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

¹H-NMR (CDCl₃) 0.89 (m, 12H) 2.05 (m, 2H) 2.46 (m, 2H) 2.62 (m, 1H) 4.20 (m, 6H) 5.11 (s, 4H) 5.30 (m, 2H) 7.35 (m, 10H)

### EXAMPLE AA-I-4

### 2-(N-CBZ-L-valyloxy)-propionic acid

### a) 4-methoxybenzyl 2-hydroxypropionate

To a stirred solution of DL -2 hydroxypropionic acid (9.0 g, 100 mmole) in 100 ml dry DMF was added potassium tert-butoxide (1 2.34 g, 110 mmole) and the mixture was stirred for one hour at 60° C. 4-methoxybenzyl chloride (18.8 g 120 mmole) was added and the mixture was stirred for eight hours at 60° C. The mixture was evaporated under reduced pressure and 250 ml ethyl acatate was added. The organic phase was washed four times with water. The organic phase was dried with sodium sulfate and concentrated in vacuo. Yield: 16.8 g

### b) 4-methoxybenzyl 2-(N-CBZ-L-valyloxy)propionate

To a solution of 4-methoxybenzyl 2-hydroxypropionate (4.2 g, 20 mmole), N-CBZ-L-valine (5.02 g, 20 mmole) and DMAP (0.24 g, 2 mmole) in 100 ml dichloromethane was added a solution of DCC (4.54 g, 22 mmole) and the mixture was stirred overnight at room temperature. The mixture was cooled to 5° C. and the urethane was filtered. The filtrate was evaporated and the product was isolated by silica gel column chromatography. Yield: 7.9 g

### c) 2-(N-CBZ-L-valyloxy)-propionic acid

To a solution of 4-methoxybenzyl 2-(N-CBZ-L-valyloxy)-propionate (7.8 g, 17.5 mmole) in dichloromethane (100 ml) was added trifluoroacetic acid (10 ml) and the solution was stirred for one hour at room temperature. The solution was evaporated under reduced pressure and the product was isolated by silica gel column chromatography. Yield: 5.0 g. The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

¹H-NMR (CDCl₃) 0.94 (m, 6H) 1.56 (d, 3H) 2.30 (m, 1H) 4.42 (m, 1H) 5.12-5.30 (m, 4H) 7.28 (m, 5H)

### EXAMPLE AA-I-5

### Succinic acid 2,3-bis-(N-CBZ-L-valyloxy)propyl ester

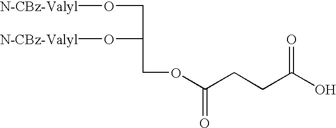

### a) 4-Methoxybenzyl succinate monoester

To a mixture of succinic anhyride (75 g, 750 mmole) and 4-methoxybenzyl alcohol (69.1 g, 500 mmole) in 1,4-dioxane (300 ml) was added pyridine (79.1 g, 1000 mmole) and the mixture was stirred for five hours at 80° C. The mixture was evaporated under reduced pressure and 600 ml of ethyl acetate and 60 ml of acetic acid were added. The organic phase was washed three times with water, dried with sodium sulfate and evaporated under reduced pressure. The product was recrystallized from toluene. Yield: 104 g.

### b) Succinic acid 2,3-dihydroxy-propyl ester, 4-methoxybenzyl ester

To a solution of glycerol (23.0 g, 250 mmole), 4-methoxybenzyl succinate monoester (5.96 g, 25 mmole) and DMAP (0.36 g, 3 mmole) in DMF (200 ml) was added DCC (6.2 g 30 mmole) and the mixture was stirred overnight at room temperature. The mixture was evaporated under reduced pressure and 150 ml dichloromethane was added. The mixture was filtered and the solution washed twice with water. The water phase was extracted two times with dichloromethane and the combined organic phases were dried with sodium sulfate. The solution was evaporated under reduced pressure and the product was isolated by silica gel column chromatography. Yield: 3.0 g

### c) Succinic acid 2,3-bis-(N-CBZ-L-valyloxy)-propyl ester, 4-methoxybenzyl ester

To a stirred solution of succinic acid 2,3-dihydroxy-propyl ester, 4-methoxybenzyl ester (2.9 g, 9.28 mmole), N-CBZ-L-valine (5.03 g, 20 mmole) and DMAP (0.244 g, 2 mmole) in dichloromethane (60 ml) was added DCC (4.5 g, 22 mmole) and the mixture was stirred overnight at room temperature. The mixture was filtered and the solution was evaporated under reduced pressure. The product was isolated by silica gel column chromatography. Yield: 2.5 g

### d) Succinic acid 2,3-bis-(N-CBZ-L-valyloxy)propyl ester

To a solution of the above intermediate (2.3 g, 2.95 mmole) in dichloromethane (25 ml) was added trifluoroacetic acid (2.5 ml) and the solution was stirred for two hours at roomtemperature. The solution was evaporated under reduced pressure and the product was isolated by silica gel column chromatography. Yield: 1,8 g. The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

¹H-NMR (CDCl₃) 0.92 (m, 12H) 2.12 (m, 2H) 2.64 (m, 4H) 4.32 (m, 4H) 5.10 (s, 4H) 5.22-5.50 (m, 3H) 7.34 (m, 10H)

### EXAMPLE AA-I-6

### Succinic acid 1,3-bis-(N-CBZ-L-valyloxy)-2-propyl ester

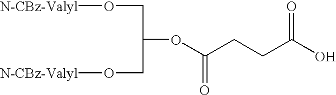

### a) Succinic acid 1,3-dibromo-2-propyl ester, 4-methoxybenzyl ester

To a solution of 1,3-dibromopropan-2-ol (21.8 g, 100 mmole), succinic acid 4-methoxybenzyl ester (28.6 g, 120 mmole) and DMAP (1.22 g, 10 mmole) in dichloromethane (400 ml) was added DCC (24.8 g, 120 mmole) in portions at about 10° C. The mixture was stirred overnight at room temperature and cooled to about 5° C. The mixture was filtered and the solution was evaporated under reduced pressure. 600 ml of ethyl acetate was added and the organic phase was washed twice with 5% acetic acid, 5% sodium hydrogen carbonate and water. The solution was dried with sodium sulfate and evaporated under reduced pressure. The product was isolated by silica gel column chromatography.Yield: 34.8 g.

### b) Succinic acid 1,3-bis-(N-CBZ-L-valyloxy)-2-propyl ester, 4-methoxybenzyl ester

To a solution of N-CBZ-L-valine (58.5 g, 232.8 mmole) in dried DMF (300 ml) was added potassium-tert.-butoxide (24.68 g, 220 mmole) and the mixture was stirred for one hour at room temperature. A solution of succinic acid 1,3-dibromo-2-propyl ester, 4-methoxybenzyl ester (34 g, 77.6 mmole) in dried DMF (50 ml) was added and the mixture was stirred for eighteen hours at 60° C. The potassium bromide was filtered and the solution was evaporated under reduced pressure. 600 ml of ethyl acetate was added and the organic phase washed twice with 5% sodium hydrogen carbonate and with water. The organic phase was dried with sodium sulfate and evaporated under reduced pressure. The product was isolated by silica gel column chromatography. Yield: 45 g

### c) Succinic acid 1,3-bis-(N-CBZ-L-valyloxy)-2-propyl ester

To a cooled solution of the intermediate immediately above (44.5 g, 57.1 mmole) in dichloromethane (500 ml) was added trifluoroacetic acid (50 ml) between 5° C. and 10° C. and the solution was stirred for two hours at 10° C. The solution was evaporated under reduced pressure and two times coevaporated with toluene. 400 ml of ethanol was added and the mixture was stirred for 30 minutes at 40° C. The mixture was cooled and the biproduct filtered. The solution was evaporated under reduced pressure and the product was isolated by silica gel column chromatography.

Yield: 33 g. The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

¹H-NMR (DMSO-d6) 0.88 (m, 12H) 2.04 (m, 2H) 2.46 (m, 4H) 3.94-4.40 (m, 6H) 5.02 (s, 4H) 5.18 (m, 1H) 7.32 (m, 10H) 7,74 (d, 2H)

### EXAMPLE M-I-7

### Alternative Route to Succinic Acid 1,3-bis-(N-CBZ-L-valyloxy)-2-propyl ester

### a) Succinic acid 1,3-dibromo-2-propyl ester, 1,1-dimethylethyl ester

To a solution of 1,3-dibromopropan-2-ol (10.9 g 50 mmole), succinic acid 1,1-dimethylethyl ester (J. Org.Chem 59 (1994) 4864) (10.45 g, 60 mmole) and DMAP (0.61 g, 5 mmole) in dichloromethane (180 ml) was added DCC (12.4 g, 60 mmole) in portions at about 10° C. The mixture was stirred overnight at room temperature and cooled to about 5° C. The mixture was filtered and the solution was evaporated under reduced pressure. 250 ml ethyl acetate was added and the organic phase was washed twice with 5% citric acid, 5% sodium hydrogen carbonate and water. The solution was dried with sodium sulfate and evaporated under reduced pressure. The product was distilled in vacuo. (bp 0,5 135-140° C.) Yield: 16.8 g

### b) Succinic acid 1,3-bis-(N-CBZ-L-valyloxy)-2-propyl ester, 1,1-dimethylethyl ester

To a solution of N-CBZ-L-valine (18.85 g, 75 mmole) in dried DMF (100 ml) was added potassium tert.-butoxide (7.85 g, 70 mmole) and the mixture was stirred for one hour at room temperature. A solution of succinic acid 1,3-dibromo-2-propyl ester, 1,1-dimethylethyl ester (9.35 g, 25 mmole) in dried DMF (20 ml) was added and the mixture was stirred for eighteen hours at 60° C. The potassium bromide was filtered and the solution evaporated under reduced pressure. 300 ml of ethyl acetate were added and the organic phase washed twice with 5% sodium hydrogen carbonate and with water. The organic phase was dried with sodium sulfate and evaporated under reduced pressure. The product was isolated by silica gel column chromatography. Yield: 14 g

### c) 1,3-bis-(N-CBZ-L-valyloxy )-2-propyl succinate monoester

To a cooled solution of succinic acid 1,3-bis-(N-CBZ-L-valyloxy)-2-propyl ester, 1,1-dimethylethyl ester (13 g, 18.18 mmole) in dichloromethane (100 ml) was added trifluoroacetic acid (20 ml) and the solution was stirred for six hours at room temperature. The solution was evaporated under reduced pressure.

200 ml ethyl acetate was added and the organic phase was washed with 5% sodium hydrogen carbonate and water. The solution was evaporated under reduced pressure. Yield: 11.7 g. The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

### EXAMPLE AA-I-8

### 3-benzyloxycarbonylpropionic acid chloromethyl ester

### a) Succinic acid monobenzyl ester

Succinic anhydride (30 g, 300 mmole) was dissolved in methylene chloride (300 ml). To the solution were added benzyl alcohol (10.2 ml, 100 mmole), 4-dimethylaminopyridine (1.22 g, 10 mmole) and pyridine (48 ml). After 3 hours the reaction mixture was poured in to citric acid aqueous solution. The organic phase was concentrated to small volume and sodium hydrogen carbonate and water were added. Then mixture was stirred for 30 min. The aqueous phase was collected, and to it was added citric acid aqueous solution. The product precipitated out, was collected and dried. 15.3 g.

### b) 3-benzyloxycarbonylpropionic acid chloromethyl ester

Succinic acid monobenzyl ester (4.16 g, 20 mmole) was dissolved in dioxane (20 ml). To the solution was added tetrabutylammonium hydroxide aqueous solution (40%, 11.6 ml, 18 mmole). The solution was dried in vacuo and coevaporated with toluene several times. The residue was dissolved in methylene chloride (60 ml) and then chloroiodomethane (14.5 ml, 200 mmole) was added to the solution. The reaction solution was stirred for 18 hr and then evaporated and the product was isolated with silica gel column chromatography. 3.64 g

### c) 3-Benzyloxycarbonylpropionic acid iodomethyl ester

3-Benzyloxycarbonylpropionic acid chloromethyl ester (2 g, 1.38 mmole) was dissolved in acetonitrile (30 ml). Sodium iodide (1.6 g, 10.9 mmole) was added to the solution. After reaction at 70° C. for 3 hr, the reaction mixture was filtered and the residue was dissolved in methylene chloride (20 ml) and refiltered. The solution was dried and gave intermediate 3-benzyloxycarbonylpropionic acid iodomethyl ester in quantitative yield. This intermediate is bonded to an accessible function of a drug, such as a ring hydroxy or carboxy function using conventional alkylation/acylation conditions as described generally herein. Following deprotection of the terminal carboxy, a di/trifunctional linker bearing R₂, such as 1,3-bis-O-(L-valyl)glycerol or iodomethyloxy-L-valyl is acylated/alkylated thereon or R₂ amide bonded thereon by conventional techniques as described herein, such as with DCC coupling agent.

### EXAMPLE AA-I-9

### 1,3-bis(N-tert-butoxycarbonyl-L-valyloxy)-2-propyl 1-iodoethyl carbonate

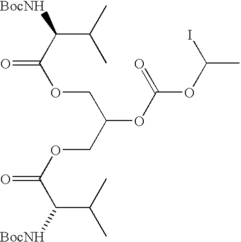

### (a) 1,3-bis(N-tert-butoxycarbonyl-L-valyloxy)-2-propyl 1-chloroethyl carbonate

To a solution of 1,3-bis(N-tert-butoxycarbonyl-L-valyloxy)-2-propanol (0.545 g, 1.11 mmol) in 5 mL dry CH₂Cl₂ were added pyridine (540 L, 6.68 mmol), with cooling and stirring in an ice bath, followed by 1-chloroethyl chloroformate (242 L, 2.22 mmol). After 1 h, the reaction mixture was diluted with 5 mL CH₂Cl₂ and washed with water (5 mL) and brine (5 mL). The organic phase was dried over anhydrous Na₂SO₄ and concentrated on a rotavapor, coevapoating several times with toluene. Column chromatogaphy (silica, 4/1 petroleum ether-ethyl acetate) gave the chloride (596 mg, 90%) as a white solid.

### (b) 1,3-bis(N-tert-butoxycarbonyl-L-valyloxy)-2-propyl 1-iodoethyl carbonate

A mixture of the chloride (596 mg, 1.0 mmol) from step (a) and Nal (684 mg, 4.57 mmol) in 10 ml dry MeCN was refluxed at 80° C. for 4 h. The reaction mixture was concentrated under vacuum and then partitioned between 30 mL diethyl ether and 10 mL water. The organic phase was washed with 5% aqueous sodium thiosulfate (2×5 mL), and the last aqueous layer was reextracted with ether (5 mL). The organic phases were combined, washed with brine, dried over Na₂SO₄, and concentrated. Flash column chromatography (silica, 4/1 petroleum ether—ethyl acetate) gave a fraction (275 mg) containing 80% iodide, as determined from ¹H NMR, and small amounts of the starting chloride and alkene from the elimination side reaction.

¹H NMR (250 MHz, CDCl₃) 0.81-0.85 (m, 6H), 0.88-0.92 (m, 6H), 1.37 (s, 18H), 2.05 (m, 2H), 2.17 (d, 3H, J=6.1 Hz), 4.12-4.46 (m, 6H), 5.00 (d, 2J=8.8 Hz), 5.11 (m, 1H), 6.68 and 6.69 (2 sets of q, 1H, J=6.1 Hz).

### EXAMPLE A-I-10

### 3-(N-benzyloxycarbonyl-L-valyloxy)-2,2-dimethylpropyl iodomethyl carbonate

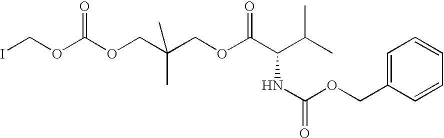

### (a) 3-(N-benzyloxycarbonyl-L-valyloxy)-2,2-dimethyl-1-propanol

A mixture of N-benzyloxycarbonyl-L-valine (2.50 g, 10.0 mmol), 2,2-dimethyl -1,3-propanediol (5.30 g, 50.9 mmol), dicyclohexylcarbodiimide (2.60 g, 12.6 mmol), and 4-dimethylaminopyridine (125 mg, 1.0 mmol) in 100 mL dry CH₂Cl₂ was stirred for 23 h. The reaction mixture was filtered and washed successively with 50 mL each of water, saturated aqueous NH₄Cl, saturated aqueous NaHCO₃, and water. The organic phase was dried over anhydrous Na₂SO₄ and concentrated. The title compound (2.99 g, 87%) was isolated by flash column chromatography (silica, 2/1 petroleum ether—ethyl acetate) as a white waxy solid.

### (b) 3-(N-benzyloxycarbonyl-L-valyloxy)-2,2-dimethylpropyl chloromethyl carbonate

Chloromethyl chloroformate (1.50 mL, 16.6 mmol) was added to a solution of the alcohol (2.74 g, 8.12 mmol) from step (a) and pyridine (4.9 mL, 61 mmol) in 40 mL dry CH₂Cl₂, in an ice bath. After stirring for 1 h, the mixture was diluted with CH₂Cl₂ and washed successively with water, saturated NaHCO₃, and brine. The organic phase was dried over anhydrous Na₂SO₄ and concentrated, coevaporating several times with toluene on a rotavapor. Flash column chromatography (silica, 2/1 petroleum ether—ethyl acetate) gave 3.31 g (95%) of the title compound.

### c) 3-(N-benzyloxycarbonyl-L-valyloxy)-2,2-dimethylpropyl iodomethyl carbonate

A mixture of the chloride (3.14 g, 7.30 mmol) from step (b) and Nal (4.37 g, 29.2 mmol) in 73 mL dry MeCN was refluxed at 80° C. for 3 h. After removal of solvent under vacuum, the mixture was partitioned between 80 mL ethyl acetate and 40 mL water. The organic phase was washed with 5% Na₂S₂O₃, and then brine, dried over anhydrous Na₂SO₄, and concentrated. Flash column chromatography (silica, petroleum ether—ethyl acetate) gave 3.68 g (97%) of the title compound.

¹H NMR (250 MHz, CDCl₃) 0.88 and 0.96 (2d, 3H each), 0.98 (s, 6H), 2.18 (m, 1H), 3.94 and 4.02 (2s, 2H each), 4.32 (dd, 1H, J=9.0, 4.7 Hz), 5.11 (s, 2H), 5.26 (d, 1H), 5.92 and 5.93 (ABq, 2H, J_{AB}=5.1 Hz), 7.35 (s, 5H).

### EXAMPLE AA-I-11

### 4-(N-Boc-L-valyloxy)butyric acid

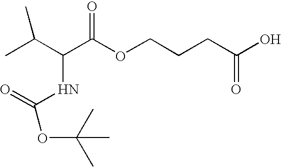

### a) Preparation of 4-bromobutyric acid benzyl ester

4-bromobutyric acid (10.6 g, 60 mmole) was dissolved in thionyl chloride (20 mml), and the reaction was kept for 4 hr. The solution was evaporated and coevaporated with toluene several times. The residue was redissolved in dichloromethane (120 ml), and then benzyl alcohol (4.14 ml, 40 mmole) was added. The solution was cooled down to −50° C. and triethylamine (10 ml, 72 mmole) was added. The reaction mixture was slowly warmed to room temperature. After 3 hr, the reaction mixture was poured into sodium bicarbonate aqueous solution and the organic phase was washed with water and dried, giving the titled product, 6.8 g.

### b) Preparation of 4-(N-Boc-L-valyloxy)butyric acid benzyl ester

N-Boc-L-valine (1.3 g, 6 mmole) was dissolved in dioxane (5 ml). To the solution was added tetrabutylammonium hydroxide aqueous solution (40%, 3.8 ml, 6 mmole), and the solution was evaporated and coevaporated with toluene several times. The residue was dissolved in DMF (15 ml) and 4-bromobutyric acid benzyl ester (1.28 g, 5 mmole) was added to it. The reaction was kept for 18 hr, and then poured into sodium bicarbonate aqueous solution and extracted with dichloromethane. The organic phase was dried and the product was isolated with silica gel column chromatography, 1.2 g.

### c) 4-(N-Boc-L-valyloxy)butyric acid

To a solution of 4-(N-Boc-L-valyloxy)butyric acid benzyl ester (1.2 g , 3 mmole) in ethyl acetate/methanol (5 ml/5 ml) was added palladium black (20 mg). The reaction mixture was kept under hydrogen at atmospheric pressure for 2 hr. The suspension was filtered through Celite and dried, giving the title product, 840 mg. The intermediate may be esterified to the phenolic hydroxy group of the NNRTI using conventional esterification (ie a coupling agent such as DCC/DMAP but is preferably used as an intermediate linking moiety in conjunction with an methyloxy moiety as described above.

¹H-NMR (CDCl₃): 5.05 (d, 1H) 4.20 (m, 3H) 2.48 (t, 2H) 2.00 (m, 2H) 1.46 (s, 9H) 0.96 (m, 6H).

### EXAMPLE AA-I-12

### N-BOC-L-isoleucine iodomethyl ester

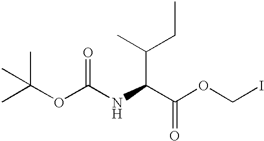

### a) N-BOC-L-isoleucine chloromethyl ester

To a solution of N-BOC-L-isoleucine (23.1 g, 0.1 mol) in dioxane (500 mL), was added dropwise a 40% aqueous solution of tetrabutylammonium hydroxide (65.6 mL, 0.1 mol). After stirring for 15 min, the solution was evaporated to dryness through co-evaporation with dioxane and toluene. The residue was dissolved in dichloromethane (500 mL) and then chloroiodomethane (72.8 mL, 1 mol) was added and the solution was stirred for 6 h at room temperature. The solution was concentrated under reduced pressure and the residue was shaken with hexane/ethyl acetate (1:1 v/v, 400 mL). The yellow crystalline solid was filtered off and the filtrate was washed with aqueous solution of sodium thiosulfate (0.1 M) and then filtered through anhydrous sodium sulfate and evaporated to dryness. The residue was column chromatographed (silica gel, 1-2% MeOH in CH₂Cl₂), to give 20.8 g of N-BOC-L-isoleucine chloromethyl ester.

### b) N-BOC-L-isoleucine iodomethyl ester

To a solution of N-BOC-L-isoleucine chloromethyl ester (19.6 g, 70 mmol) in acetonitrile (300 mL), was added sodium iodide (31.5 g, 210 mmol). The solution was stirred for 4 h at 60° C. The resulting suspension was filtered and the filtrate was evaporated. The residue was dissolved in CH₂Cl₂ and washed with aqueous sodium thiosulfate (0.1 M). The organic phase was dried (Na₂SO₄) and concentrated under reduced pressure. The crude product was column chromatographed (silica gel, 2% MeOH in CH₂Cl₂), to give 22.6 g of N-BOC-L-isoleucine iodomethyl ester.

¹H-NMR (CDCl₃): 6.04 (d, 1H), 5.82 (d, 1H), 4.97 (d, 1H), 4.25 (dd, 1H), 1.98-1.80 (m, 1H), 1.43 (s, 9H), 1.50-1.05 (m, 2H), 0.97-0.88 (m, 6H).

### EXAMPLE AA-I-13

### 2,2-dimethyl-3-(N-Boc-L-valyloxy)propionic acid iodomethyl ester

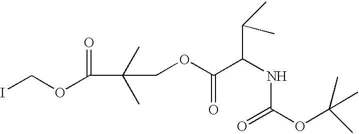

### a) 2,2-dimethyl-3-(N-Boc-L-valyloxy)propionic acid

N-Boc-L-valine (10.8 g, 50 mmole), 4-dimethylaminopyridine (610 mg, 5 mmole) and DCC (6.18 g, 30 mmole) were dissolved in methylene chloride (100 ml). After stirring for 2 hour the mixture was filtered. To the filtrate were added 2,2-dimethyl-3-hydroxy-propionic acid (3.54 g, 30 mmole) and pyridine (10 ml). After 18 hr, the reaction mixture was filtered, and the filtrate was poured into sodium hydrogen carbonate aqueous solution, the organic phase was then washed with citric acid aqueous solution and water succesively. After evaporation the product was isolated with silica gel column chromatography to yield 4.4 g.

### b) 2,2-dimethyl-3-(N-Boc-L-valyloxy)propionic acid chloromethyl ester

2,2-dimethyl-3-(N-Boc-L-valyloxy)propionic acid (3.9 g, 12.3 mmole) was dissolved in dioxane (60 ml). To the solution was added tetrabutylammonium hydroxide aqueous solution (40%, 7.78 ml, 12 mmole). The solution was dried in vacuo, and it was coevaporated with toluene for several times. The residue was dissolved in methylene chloride and then chloroiodomethane (18.9 ml, 260 mmole) was added to the solution. After 18 hr, the reaction solution was evaporated and the product was isolated with silica gel column chromatography to yield 3.7 g.

### c) 2,2-dimethyl-3-(N-Boc-L-valyloxy)propionic acid iodomethyl ester

2,2-Dimethyl-3-(N-Boc-L-valyloxy)propionic acid chloromethyl ester (3.6 g, 10 mmole ) was dissolved in acetonitrile (50 ml). Sodium iodide (2.1 g, 14 mmole) was added to the solution. After reaction at 70° C. for 2 hr, the reaction mixture was filtered and the residue was dissolved in methylene chloride (20 ml) and refiltered. The solution was dried and gave 4.34 g of the titled product.

¹H-NMR (CDCl₃): 5.92 (dd, 2H) 5.10 (d, 1H) 4.24 (m, 1H) 4.15 (dd, 2H) 2.01 (m, 1H) 1.44 (s, 9H) 1.25 (d, 6H) 0.91 (m, 6H)

### EXAMPLE AA-I-14

### 3,3-bis(N-CBz-L-valyloxymethyl)-propionic acid iodomethyl ester

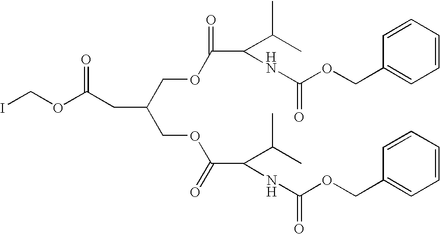

### a) Preparation of 3,3-bis(N-CBz-L-valyloxymethyl)-propionic acid chloromethyl ester

3,3-bis(N-CBz-L-valyloxymethyl)-propionic acid (3 g, 5 mmole) was dissolved in dioxane (20 ml). To the solution was added tetrabutylammonium hydroxide aqueous solution (40%, 3.11 ml, 4.8 mmole). The solution was dried in vacuo, and it was coevaporated with toluene several times. The residue was dissolved in methylene chloride (15 ml) and then chloroiodomethane (7.3 ml, 100 mmole) was added to the solution. The reaction solution was refluxed for 18 hr and then evaporated and the product was isolated with silica gel column chromatography. 900 mg.

### b) 3,3-bis-(N-CBz-L-valyloxymethyl)propionic acid iodomethyl ester

3,3-bis(N-CBz-L-valyloxymethyl)-propionic acid chloromethyl ester (900 mg, 1.38 mmole) was dissolved in acetonitrile (5 ml). Sodium iodide (289 mg, 1.93 mmole) was added to the solution. After reaction at 70° C. for 3 hr, the reaction mixture was filtered and the residue was dissolved in methylene chloride (5 ml) and refiltered. The solution was dried and gave the titled product. 800 mg.

¹H-NMR (CDCl₃): 7.35 (m, 10 H) 5.88 (dd, 2H) 5.25 (d, 2H) 4.29 (m, 2H) 4.18 (m, 4H) 2.56 (m, 1H) 2.42 (d, 2H) 2.16 (m, 2H) 0.93 (m 12 H)

### EXAMPLE AA-I-15

### 2-(N-CBz-L-valyloxy)ethoxycarbonyloxymethyl iodide

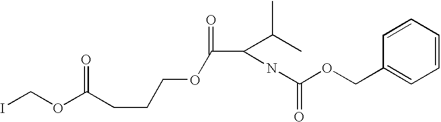

2-(N-CBz-L-valyloxy)ethoxycarbonyloxymethyl chloride (1.16 g, 3 mmole) was dissolved in acetonitrile (10 ml). Sodium iodide (630 g, 4.2 mmole) was added to the solution. After reaction at 65° C. for 2.5 hr, the reaction mixture was cooled down to room temperature and filtered and the residue was dissolved in methylene chloride (5 ml) and refiltered. The solution was dried and gave the titled product. 1.2 g.

¹H-NMR (CDCl₃): 7.35 (m, 5H) 5.93 (dd, 2H) 5.26 (d, 1H) 5.11 (s, 2H) 4.39 (m, 5H) 2.18 (m, 1H) 0.94 (m, 6 H).

### EXAMPLE AA-I-16

### 1,3-bis(N-tert-butoxycarbonyl-L-valyloxy)-2-propyl iodomethyl carbonate)

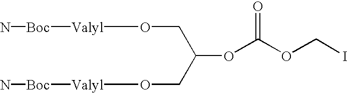

### a) 1-O-(N-tert-butoxycarbonyl-L-valyl)glycerol

N-tert-Butoxycarbonyl-L-valine (32.53 g, 0.150 mol), N,N′-dicyclohexyl-carbodiimide (37.85 g, 0.183 mol, and 4-dimethylaminopyridine (1.83 g, 0.015 mol) were added to glycerol (138.12 g., 1.5 mol) in 500 mL dry DMF and the mixture was stirred at rt under N₂ for 3 days. The reaction mixture was filtered, concentrated under vacuum, and then partitioned between 300 mL EtOAc and 150 mL H₂O. The aqueous phase was reextracted with 150 mL EtOAc. The organic phases were combined and washed successively with 100 mL each of saturated aqueous NaHCO₃, saturated NH₄Cl, and brine. Drying over anhydrous Na₂SO4, and concentration under vacuum gave a viscous light yellow oil as crude product. Flash column chromatography on silica gel with 4/1 EtOAc-petroleum ether (BP 40-60° C.) gave 18.27 g (42%) of product (alternative nomenclature: 3-(N-tert-butoxycarbonyl-L-valyloxy)-1,2-propanediol). Reactions done overnight gave similar yields.

### b) 1,3-di-O-(N-tert-butoxycarbonyl-L-valyl)glycerol

1-O-(N-tert-butoxycarbonyl-L-valyl)glycerol (17.95 g. 61.6 mmol), Boc-L-valine (6.69 g, 30.8 mmol), DMAP (0.38 g, 3.1 mmol), and DCC (7.10 g, 34.4 mmol) in 240 mL CH₂Cl₂ and 60 mL DMF were stirred at rt under N₂ for 18 h. The reaction mixture was filtered, concentrated under vacuum, and redissolved in 200 mL EtOAc. The organic solution was washed with 50 mL saturated NH₄Cl. The aqueous phase was reextracted with 50 mL EtOAc. The organic phases were combined, washed successively with 50 mL saturated NaHCO₃ and 50 mL brine, dried over Na₂SO₄, and concentrated under vacuum. Flash column chromatography of the crude material on silica gel (eluent 2/1 petroleum ether—EtOAc, and then EtOAc) gave 7.41 g (49%) of the title compound (alternative nomenclature: 1,3-bis(N-tert-butoxycarbonyl-L-valyloxy)-2-propanol).

### c) 2-O-chloromethoxycarbonyl-1,3-di-O-(N-tert-butoxycarbonyl-L-valyl)glycerol

Chloromethyl chloroformate (2.70 mL, 30 mmol) was added to a solution of 1,3-di-O-(N-tert-butoxycarbonyl-L-valyl)glycerol (7.27 g, 14.8 mmol) and pyridine (7.2 mL, 89 mmol) in 60 mL dry CH₂Cl₂, in an ice bath, under N₂. After stirring for 1 h 45 min, the reaction mixture was diluted with 100 mL CH₂Cl₂ and washed with 40 mL water. The aqueous phase was reextracted with 20 mL H₂O. The organic phases were combined, washed with 40 mL saturated NaHCO₃, followed by 2×50 mL brine, dried over Na₂SO₄, and concentrated under vacuum. Flash column chromatography on silica gel with 2/1 hexane-EtOAc gave 8.03 g (93%) of the title compound (alternative nomenclature:1,3-bis(N-tert-butoxycarbonyl-L-valyloxy)-2-propyl chloromethyl carbonate).

### d) 2-O-iodomethoxycarbonyl-1,3-di-O-(N-tert-butoxycarbonyl-L-valyl)glycerol

A solution of 2-O-chloromethoxycarbonyl-1,3-di-O-(N-tert-butoxycarbonyl-L-valyl)propane-1,2,3-triol (7.86 g, 13.5 mmol) and Nal (8.09 g, 54.0 mmol) in 135 mL dry acetonitirile was refluxed at 80° C. for 4 h under N₂. The reaction mixture was concentrated under vacuum, and then partitioned between 150 mL diethyl ether and 50 mL H₂O. The aqueous layer was reextracted with 2×25 mL ether. The combined organic phases were washed successively with 25 mL aqueous Na₂S₂O₃ and 50 mL brine, dried over Na₂SO₄, and concentrated. Flash column chromatography (silica gel, 2/1 hexane-ethyl acetate gave 8.38 g (92%) title product (alternative name: 2-iodomethoxycarbonyloxy-1,3-bis-(N-tert-butoxycarbonyl-L-valyloxy)propane or 1,3-bis(N-tert-butoxycarbonyl-L-valyloxy)-2-propyl iodomethyl carbonate) as a white solid.

¹H NMR (250 MHz, CDCl₃) 0.81 (d, 6H), 0.88 (m, 6H), 1.36 (s, 18H), 2.06 (m, 2H), 4.11-4.46 (m, 6H), 5.0 (b rd, 2H), 5.12 (m, 1H), 5.88 (s, 2H).

### EXAMPLE A-I-1

### Iodomethyl 2-methyl-2-(N-benzyloxycarbonyl-L-valyloxymethyl) propionate

### a) 4-Methoxybenzyl 2-(hydroxymethyl)-2-methyl propionate

2-(Hydroxymethyl)-2-methyl propionic acid was esterified by alkylation with 4-methoxybenzyl chloride by conventional means, namely treatment with aqueous NaOH, followed by evaporation and dissolution in an organic solvent such as DMF to which the 4-methoxybenzyl chloride is added and the reaction warmed and agitated, such as stirring at 60° C. for one hour. The reaction mixture is cooled, concentrated by rotavapor and the resulting concentrated suspension partitioned between water and dichloromethane. The organic phase is evaporated and the reside subjected to silica gel column chromatography, for example with 0, 2, 4% EtOH in dichloromethane to yield the title compound (7.10 g). R_{f} (2%MeOH/CHCl₃) 0.40.

### b) 4-Methoxybenzyl 2-(N-benzyloxycarbonyl-L-valyloxymethyl)-2-methyl propionate

4-Methoxybenzyl 2-(hydroxymethyl)-2-methyl propionate (2.50 g, 10.5 mmol), N-benzyloxy carbonyl-L-valine (2.51 g, 10 mmole), 4-dimethylaminopyridine (183 mg) and 1-hydroxybenzotriazole (1.35 g, 10 mmole) were mixed and dissolved in N,N-dimethylformamide (90 ml). Then dicyclohexyl-carbodiimide (2.47 g 12 mmol) was added. After stirring for 3 days at room temperature the suspension was filtered and the filtrate evaporated in vacuo. The residue was partitioned between 0.1 M citric acid and dichloromethane. The organic phase was then extracted with aqueous saturated NaHCO₃ and evaporated in vacuo. The residue was silica gel column chromatographed (0, 1, 2, 3% ethanol in dichloromethane). The appropriate fractions were pooled and evaporated in vacuo to give the title compound (2.72 g).

### d) 2-(N-benzyloxycarbonyl-L-valyloxymethyl)-2-methyl propionic acid

To a solution of 4-methoxybenzyl 2-(N-benzyloxycarbonyl -L-valyloxymethyl)-2-methyl propionate (2.72 g, 5.76 mmole), was added trifluoroacetic acid (11.5 ml) and the emerging dark red solution was stirred for 30 min at room temperature. The solution was evaporated to dryness with dioxane and toluene. The residue was silica gel column chromatographed (2, 3, 4% ethanol in dichloromethane). The appropriate fractions were pooled and evaporated in vacuo to give the title compound (1.86 g).

R_{f} (2%MeOH/CHCl₃) 0.30.

¹H-NMR (CDCl₃): 7.32 (s, 5H), 5.32 (d, 1H), 5.10 (s, 2H), 4.32 (d,d, 1H), 4.21 (d,d, 2H), 2.13 (m, 1H), 1.26 (s, 3H), 1.25 (s, 3H), 0.95 (d, 3H), 0.86 (d, 3H).

### c) Chloromethyl 2-(N-benzyloxycarbonyl-L-valyloxymethyl)-2-methyl propionate

2-(N-benzyloxycarbonyl-L-valyloxymethyl)-2-methyl propionic acid was esterified by conventional techniques, namely dissolution in an organic solvent such as dioxane and dropwise addition of aqueous tetrabutylammonium hydroxide, followed by evaporation. The residue is dissolved in dichloromethane and then chloroiodomethane and the mixture stirrred for 6 hours at room temperature, followed by partition, shaking the filtrate with aqueous sodium thiosulphate. 0.1M, filtration and evaporation. The title compound (1.40 g) was obtained after silica gel column chromatography (0, 1, 2, 3% ethanol in dichloromethane).

### d) Iodomethyl 2-(N-benzyloxycarbonyl-L-valyloxymethyl)-2-methyl propionate

Chloromethyl 2-(N-benzyloxycarbonyl-L-valyloxymethyl)-2-methyl propionate was converted to iodide by conventional techniques, namely addition to NaI in acetonitrile, stirring and heating, for instance to 75° C. for four hours. The resulting suspension is filtered and the filtrate evaporated, dissolved in organic solvent such as toluene and shaken with aqueous sodium thiosulphate (0.1M) and evaporation to give the title compound (1.25 g) practically pure.

R_{f} (2%MeOH/CHCl₃) 0.80.

¹H-NMR (CDCl₃): 7.35 (s, 5H), 5.90 (d,d, 2H), 5.24 (d, 1H), 5.10 (s, 2H), 4.31 (d,d, 1H), 4.14 (d,d, 2H), 2.16 (m, 1H), 1.22 (s, 6H), 0.96 (d, 3H), 0.87 (d, 3H).

### EXAMPLE A-I-2

### Iodomethyl 2-(N-benzyloxycarbonyl-L-valyloxy)-DL-propionate

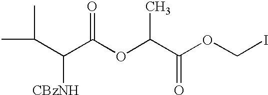

### a) Chloromethyl 2-(N-benzyloxycarbonyl-L-valyloxy)-DL-propionate

2-(N-benzyloxycarbonyl-L-valyloxy) propionic acid (1 g) was esterified by the method described in Example A-I-I, step d. The title compound (0.76 g) was obtained after silica gel column chromatography (0, 1% ethanol in dichloromethane). R_{f} (2%MeOH/CHCl₃) 0.75.

### b) Iodomethyl 2-(N-benzyloxycarbonyl-L-valyloxy)-DL-propionate

Chloromethyl 2-(N-benzyloxycarbonyl-L-valyloxymethyl)-2-methyl propionate was converted to iodide by the method described in Example A-I-1, step e to give the title compound (0.95 g) practically pure. Rf (2%MeOH/CHCl₃) 0.75. ¹H-NMR (CDCl₃): 7.33 (s, 5H), 5.98 (d, 1H), 5.86 (d, 1H), 5.26 (d, 1H), 5.10 (s, 2H), 5.07 (q, 1H), 4.38 (d,d, 1H), 2.30 (m, 1H), 1.49 (d, 3H), 1.03 (d, 3H), 0.95 (d, 3H).

### EXAMPLE A-I-3

### Iodomethyl (1,3-di-(N-benzyloxycarbonyl)-L-valyloxy)-2-propyl carbonate

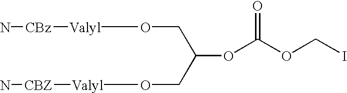

### a) Chloromethyl (1,3-di-(N-benzyloxycarbonyl)-L-valyloxy)-2-propyl carbonate

To a solution of 1,3-di-((N-benzyloxycarbonyl)-L-valyloxy)propan-2-ol (1.34 g, 2.4 mmole) in dichloromethane (10 ml) was added dry pyridine (1.15 ml, 14.4 mmol) and chloromethyl chloroformate (0.43 ml, 4.8 mmole) at 0° C. The reaction was then stirred for 30 min and then poured into aqueous 50% saturated sodium chloride/0.1M citric acid solution and extracted with dichloromethane. The organic phase was evaporated and the residue silica gel column chromatographed (0, 1, 1.5% ethanol in dichloromethane). The appropriate fractions were pooled and evaporated in vacuo to give the title compound (1.26 g). R_{f} (2%MeOH/CHCl₃) 0.85.

### b) Preparation of iodomethyl (1,3-di-(N-benzyloxycarbonyl)-L-valyloxy)-2-propyl carbonate

Chloromethyl (1,3-di-(N-benzyloxycarbonyl)valyloxy)-2-propyl carbonate was converted to iodide by the method described in Example A-I-1, step e) to give the title compound (1.37 g) practically pure.

R_{f} (2%MeOH/CHCl₃) 0.85.

¹H-NMR (CDCl₃): 7.34 (s, 10H), 5.93 (d, 1H), 5.89 (d, 1H), 5.21 (m, 3H), 5.11 (s, 4H), 4.50-4.17 (m, 6H), 2.12 (m, 2H), 0.97 (d, 6H), 0.88 (d, 6H).

### EXAMPLE A-I-4

### Iodomethyl 2-(N-benzyloxycarbonyl-L-valyloxy)isobutyrate

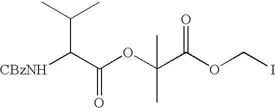

### a) 4-Methoxybenzyl 2-hydroxyisobutyrate

2-hydroxy isobutyric acid (1.56 g) was esterified by alkylation with 4-methoxybenzyl chloride by the method described in Example A-I-1, step a). The title compound (2.65 g) was obtained after silica gel column chromatography (0, 1, 2% ethanol in dichloromethane). R_{f} (2%MeOH/CHCl₃) 0.45.

### b) 4-Methoxybenzyl 2-(N-benzyloxycarbonyl-L-valyloxy) isobutyrate

4-methoxybenzyl 2-hydroxyisobutyrate was acylated with N-benzyloxycarbonyl-L-valine by the method described in Example A-I-1, step b). The title compound (3.21 g) was obtained after silica gel column chromatography (0, 1, 1.5% ethanol in dichloromethane). R_{f} (2%MeOH/CHCl₃) 0.70.

### c) 2-(N-benzyloxycarbonyl-L-valyloxy) isobutyric acid

4-methoxybenzyl 2-(N-benzyloxycarbonyl-L-valyloxy) isobutyrate was de-esterified by the method described in Example A-I-1 step c. The title compound (2.01 g) was obtained after silica gel column chromatography (2, 10, 20% ethanol in dichloromethane). R_{f} (2%MeOH/CHCl₃) 0.30.

¹H-NMR (CDCl₃): 7.32 (s, 5H), 5.33 (d, 1H), 5.10 (s, 2H), 4.31 (d,d, 1H), 2.22 (m, 1H), 1.57 (s, 6H), 0.98 (d, 3H), 0.89 (d, 3H).

### d) Chloromethyl 2-(N-benzyloxycarbonyl-L-valyloxy) isobutyrate

2-(N-benzyloxycarbonyl-L-valyloxy) isobutyric acid was esterified by the method described in Example A-I-1, step d. The title compound (1.90 g) was obtained after silica gel column chromatography (0, 1, 1.5% ethanol in dichloromethane). R_{f} (2%MeOH/CHCl₃) 0.80.

### e) Iodomethyl 2-(N-benzyloxycarbonyl-L-valyloxy) isobutyrate

Chloromethyl 2-(N-benzyloxycarbonyl-L-valyloxy) isobutyrate was converted to iodide by the method described in Example A-I-1, step e to give the title compound (2.32 g) practically pure.

R_{f} (2%MeOH/CHCl₃) 0.80.

¹H-NMR (CDCl₃): 7.33 (s, 5H), 5.89 (s, 2H), 5.22 (d, 1H), 5.11 (s, 2H), 4.29 (d,d, 1H), 2.21 (m, 1H), 1.55 (s, 3H), 1.53 (s, 3H), 1.00 (d, 3H), 0.93 (d, 3H).

### EXAMPLE A-I-5

### Iodomethyl 2-(N-benzyloxycarbonyl-L-valyloxy)-3-methyl-(S)-(+)-butyrate

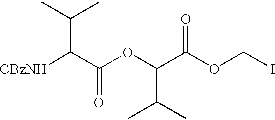

### a) 4-Methoxybenzyl 2-hydroxy-3-methyl-(S)-(+)-butyrate

2-hydroxy-3-methyl-(S)-(+)-butyric acid (1.77 g) was esterified by alkylation with 4-methoxybenzyl chloride by the method described in Example A-I-1, step a. The title compound (3.10 g) was obtained after silica gel column chromatography (0, 1, 2% ethanol in dichloromethane). R_{f} (2%MeOH/CHCl₃) 0.50.

### b) 4-Methoxybenzyl 2-(N-benzyloxycarbonyl-L-valyloxy)-3-methyl-(S)-(+)-butyrate

4-Methoxybenzyl 2-hydroxy-3-methyl-(S)-(+)-butyrate was acylated with N-benzyloxycarbonyl-L-valine by the method described in Example A-I-1, step b. The title compound (5.74 g) was obtained after silica gel column chromatography (0, 1, 1.5% ethanol in dichloromethane). R_{f} (2%MeOH/CHCl₃) 0.70.

### c) 2-(N-benzyloxycarbonyl-L-valyloxy)-3-methyl-(S)-(+)-butyric acid

4-methoxybenzyl 2-(N-benzyloxycarbonyl-L-valyloxy)-3-methyl-(S)-(+)-butyrate was de-esterified by the method described in Example A-I-1, step c. The title compound (3.41 g) was obtained after silica gel column chromatography (2, 10, 20% ethanol in dichloromethane). R_{f} (2%MeOH/CHCl₃) 0.45. ¹H-NMR (CDCl₃): 7.36 (s, 5H), 5.38 (d, 1H), 5.11 (s, 4H), 4.90 (d, 1H), 4.41 (d,d, 1H), 2.28 (m, 2H), 1.04-0.89 (m, 12H).

### d) Chloromethyl 2-(N-benzyloxycarbonyl-L-valyloxy)-3-methyl-(S)-(+)-butyrate

2-(N-benzyloxycarbonyl-L-valyloxy)-3-methyl-(S)-(+)-butyric acid was esterified by the method described in Example A-I-1, step d. The title compound (2.96 g) was obtained after silica gel column chromatography (0, 1, 2% ethanol in dichloromethane). R_{f} (2%MeOH/CHCl₃) 0.85.

### e) Iodomethyl 2-(N-benzyloxycarbonyl-L-valyloxy)-3-methyl-(S)-(+)-butyrate

Chloromethyl 2-(N-benzyloxycarbonyl-L-valyloxy)-3-methyl-(S)-(+)-butyrate was converted to iodide by the method described in Example A-I-1, step e to give the title compound (3.64 g) practically pure.

R_{f} (2%MeOH/CHCl₃) 0.85.

¹H-NMR (CDCl₃): 7.36 (s, 5H), 6.00 (d, 1H), 5.83 (d, 1H), 5.28 (d, 1H), 5.11 (s,4H), 4.83 (d, 1H), 4.41 (d,d, 1H), 2.29 (m, 2H), 1.05-0.90 (m, 12H).

### EXAMPLE A-I-6

### Iodomethyl 2-(N-benzyloxycarbonyl-L-valyloxy)-2-phenyl-DL-acetate

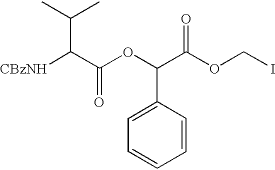

### a) 4-Methoxybenzyl 2-hydroxy-2-phenyl-DL-acetate

DL-mandelic acid (2.28 g) was esterified by alkylation with 4-methoxybenzyl chloride by the method described in Example A-I-1, step a. The title compound (3.69 g) was obtained after silica gel column chromatography (0, 1, 1.5% ethanol in dichloromethane). R_{f} (2%MeOH/CHCl₃) 0.55.

### b) 4-Methoxybenzyl 2-(N-benzyloxycarbonyl-L-valyloxy)-2-phenyl-DL-acetate

4-Methoxybenzyl 2-hydroxy-2-phenyl-DL-acetate was acylated with N-benzyloxycarbonyl-L-valine by the method described in Example A-I-1, step b. The title compound (6.50 g) was obtained after silica gel column chromatography (0, 1, 1.5% ethanol in dichloromethane). R_{f} (2%MeOH/CHCl₃) 0.75.

### c) 2-(N-benzyloxycarbonyl-L-valyloxy)-2-phenyl-DL-acetic acid

4-Methoxybenzyl 2-(N-benzyloxycarbonyl-L-valyloxy)-2-phenyl-DL-acetate was de-esterified by the method described in Example A-I-1, step c. The title compound (4.75 g) was obtained after silica gel column chromatography (2, 10, 20% ethanol in dichloromethane). R_{f} (2%MeOH/CHCl₃) 0.40.

¹H-NMR (CDCl₃): 7.36 (m, 10H), 5.91 (d, 1H), 5.27 (m, 1H), 5.04 (s, 2H), 4.57-4.40 (2xd,d, 1H), 2.30 (m, 1H), 1.01-0.82 (m, 6H).

### d) Chloromethyl 2-(N-benzyloxycarbonyl-L-valyloxy)-2-phenyl-DL-acetate

2-(N-benzyloxycarbonyl-L-valyloxy)-2-phenyl-DL-acetic acid was esterified by the method described in Example A-I-1, step d. The title compound (1.69 g) was obtained after silica gel column chromatography (0, 1, 2% ethanol in dichloromethane). R_{f} (2%MeOH/CHCl₃) 0.80.

### e) Iodomethyl 2-(N-benzyloxycarbonyl-L-valyloxy)-2-phenyl-DL-acetate

Chloromethyl 2-(N-benzyloxycarbonyl-L-valyloxy)-2-phenyl-DL-acetate was converted to iodide by the method described in Example A-I-1, step e to give the title compound (1.89 g) practically pure.

R_{f} (2%MeOH/CHCl₃) 0.80.

¹H-NMR (CDCl₃): 7.36 (m, 10H), 5.94-5.82 (m, 3H), 5.28 (m, 1H), 5.10 (s, 2H), 4.46 (m, 1H), 2.21 (m, 1H), 1.08-0.85 (m, 6H).

### EXAMPLE A-I-7

### Iodomethyl 4-(N-benzyloxycarbonyl-L-valyloxy)benzoate

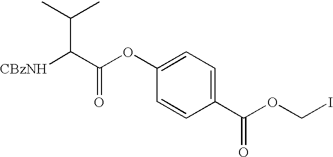

### a) 4-Methoxybenzyl 4-hydroxybenzoate

4-Hydroxybenzoic acid (1.38 g) was esterified by alkylation with 4-methoxybenzyl chloride by the method described in Example A-I-1, step a. The title compound (2.06 g) was obtained after silica gel column chromatography (0, 1, 2, 3% ethanol in dichloromethane). R_{f} (2%MeOH/CHCl₃) 0.40.

### b) 4-Methoxybenzyl 4-(N-benzyloxycarbonyl-L-valyloxy)benzoate

4-Methoxybenzyl 4-hydroxybenzoate was acylated with N-benzyloxycarbonyl-L-valine by the method described in Example A-I-1, step b. The title compound (2.71 g) was obtained after silica gel column chromatography (0, 1% ethanol in dichloromethane). R_{f} (2%MeOH/CHCl₃) 0.70.

### c) 4-(N-benzyloxycarbonyl-L-valyloxy)benzoic acid

4-Methoxybenzyl 4-(N-benzyloxycarbonyl-L-valyloxy)benzoate was de-esterified by the method described in Example A-I-1, step c. The title compound (1.86 g) was obtained after silica gel column chromatography (2, 10, 20% ethanol in dichloromethane). R_{f} (2%MeOH/CHCl₃) 0.20.

¹H-NMR (CDCl₃): 8.15 (d, 2H), 7.34 (m, 5H), 7.22 (d, 2H), 5.38 (d, 1H), 5.17 (s, 2H), 4.58 (d,d, 1H), 2.34 (m, 1H), 1.12 (s, 3H), 0.96 (d, 3H).

### d) Chloromethyl 4-(N-benzyloxycarbonyl-L-valyloxy)benzoate

4-(N-benzyloxycarbonyl-L-valyloxy) benzoic acid was esterified by the method described in Example A-I-1, step d. The title compound (0.95 g) was obtained after silica gel column chromatography (0, 1% ethanol in dichloromethane). R_{f} (2%MeOH/CHCl₃) 0.80.

### e) Iodomethyl 4-(N-benzyloxycarbonyl-L-valyloxy)benzoate

Chloromethyl 4-(N-benzyloxycarbonyl-L-valyloxy)benzoate was converted to iodide by the method described in Example A-I-1, step e to give the title compound (1.16 g) practically pure.

R_{f} (2%MeOH/CHCl₃) 0.80.

¹H-NMR (CDCl₃): 8.11 (d, 2H), 7.35 (m, 5H), 7.21 (d, 2H), 6.15 (s, 2H), 5.32 (d, 1H), 5.14 (s, 2H), 4.55 (d,d, 1H), 2.34 (m, 1H), 1.10 (s, 3H), 1.03 (d, 3H).

### EXAMPLE A-I-8

### Iodomethyl 5-(N-CBz-L-valyloxy)-2 2-dimethylvalerate

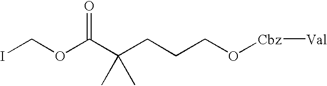

### a) 4-Methoxybenzyl 2,2-dimethyl-4-pentenoate

To a solution of 2,2-dimethyl-4-pentenoic acid (11.5 g, 90 mmol) in DMF (250 mL) at room temperature, was added potassium tert-butoxide (11.1 g, 99 mmol). The reaction mixture was stirred at 60° C. for 1 h. 4-Methoxybenzyl chloride (16.9 g, 108 mmol) was added and the reaction mixture was stirred at 60° C. for 4h. The DMF was evaporated under vacuum, the residue was dissolved in ether (500 mL) and washed with water (3×200 mL). The organic phase was dried with Na₂SO₄ and evaporated to give 21.4 g of 4-methoxybenzyl 2,2-dimethyl-4-pentenoate.

### b) 4-Methoxybenzyl 5-hydroxy-2,2-dimethylvalerate

A mixture of 4-methoxybenzyl 2,2-dimethyl-4-pentenoate (9.50 g, 38 mmol) and 9-BBN (115 mL, 57 mmol, 0.5 M in THF) was stirred at 60° C. for 60 min, whereupon the reaction mixture was cooled to −5° C. H₂O (35 mL) was added, the reaction mixture was stirred for 5 min at −5° C., an aqueous solution of NaOH (35 mL, 3M) was added and the reaction mixture was stirred for a further 10 min at −5° C. An aqueous solution of H₂O₂ (35 mL, 30%) was added dropwise and the temperature of the reaction mixture was allowed to assume room temperature, whereupon the reaction mixture was stirred for 30 min at room temperature. After evaporation, water (200 mL) was added and the resulting mixture was extracted with CH₂Cl₂ (5×200 mL). The combined organic layers were dried (Na₂SO₄) and concentrated under reduced pressure. The crude product was column chromatographed (silica gel, 1→8% MeOH in CH₂Cl₂), to give 6.32 g of 4-methoxybenzyl 5-hydroxy-2,2-dimethylvalerate.

### c) 4-Methoxybenzyl 5-(N-CBz-L-valyloxy)-2,2-dimethylvalerate

To a mixture of DCC (9.41 g, 46 mmol), DMAP (0.586 g, 4.8 mmol) and N-CBz-L-valine (12.1 g, 48 mmol) in CH₂Cl₂ (200 mL) at 0° C., was added dropwise a solution of 4-methoxybenzyl 5-hydroxy-2,2-dimethyl-valerate (6.40 g, 24 mmol) in CH₂Cl₂ (50 mL). After 1 h at 0° C., the temperature of the reaction mixture was allowed to assume room temperature and then the mixture was stirred for 5 h at room temperature. The mixture was filtered through a glass filter and the solvent was removed under reduced pressure. The crude product was column chromatographed (silica gel, 1→4% MeOH in CH₂Cl₂), to give 8.61 g 4-methoxybenzyl 5-(N-CBz-L-valyloxy)-2,2-dimethylvalerate.

### d) 5-(N-CBz-L-valyloxy)-2,2-dimethylvaleric acid

To a solution of 4-methoxybenzyl 5-(N-CBz-L-valyloxy)-2,2-dimethylvalerate (8.24 g, 16.5 mmol) in CH₂Cl₂ (100 mL) at room temperature, was added trifluoroacetic acid (5 mL). After 1 h at room temperature, the reaction mixture was concentrated under reduced pressure. The crude product was column chromatographed (silica gel, 3→5% MeOH in CH₂Cl₂), to give 6.00 g of 5-(N-CBz-L-valyloxy)-2,2-dimethylvaleric acid.

¹H-NMR (CDCl₃): 10.94 (br s,1H), 7.35 (s, 5H), 5.45 (d, 1H), 5.11 (s, 2H), 4.30 (dd, 1H), 4.21-4.00 (m, 2H), 2.28-2.07 (m, 1H), 1.68-1.51 (m, 4H), 1.21 (s, 6H), 0.97 (d, 3H), 0.89 (d, 3H).

### e) Chloromethyl 5-(N-CBz-L-valyloxy)-2,2-dimethylvalerate

To a solution of 5-(N-CBz-L-valyloxy)-2,2-dimethylvaleric acid (5.88 g, 15.5 mmol) in dioxane (100 mL), was added dropwise a 40% aqueous solution of tetrabutylammonium hydroxide (10.1 g). After stirring for 5 min, the solution was evaporated to dryness through co-evaporation with dioxane and toluene. The residue was dissolved in dichloromethane (100 mL) and then chloroiodomethane (11.3 mL, 155 mmol) was added and the solution was stirred for 6 h at room temperature. The solution was concentrated under reduced pressure and the residue was shaken with hexane/ethyl acetate (1:1 v/v, 200 mL). The yellow crystalline solid was filtered off and the filtrate was washed with aqueous solution of sodium thiosulfate (0.1 M) and the filtered through anhydrous sodium sulfate and evaporated to dryness. The residue was column chromatographed (silica gel, 1-4% MeOH in CH₂Cl₂), to give 3.95 g of chloromethyl 5-(N-CBz-L-valyloxy)-2,2-dimethylvalerate.

### f) Iodomethyl 5-(N-CBz-L-valyloxy)-2,2-dimethylvalerate

To a solution of chloromethyl 5-(N-CBz-L-valyloxy)-2,2-dimethylvalerate (3.85 g, 9 mmol) in acetonitrile (50 mL), was added sodium iodide (5.40 g, 36 mmol). The solution was stirred for 4 h at 60° C. The resulting suspension was filtered and the filtrate was evaporated. The residue was dissolved in CH₂Cl₂ and washed with aqueous sodium thiosulfate (0.1 M). The organic phase was dried (Na₂SO₄) and concentrated under reduced pressure. The crude product was column chromatographed (silica gel, 1% MeOH in CH₂Cl₂), to give 4.26 g of iodomethyl 5-(N-CBz-L-valyloxy)-2,2-dimethylvalerate.

¹H-NMR (CDCl₃): 7.34 (s, 5H), 5.90 (s, 2H), 5.32 (d, 1H), 5.10 (s, 2H), 4.29 (dd, 1H), 4.18-4.02 (m, 2H), 2.26-2.08 (m, 1H), 1.65-1.50 (m, 4H), 1.17 (s, 6H), 0.97 (d, 3H), 0.89 (d, 3H).

### EXAMPLE A-I-9

### 2-(N-CBz-L-valyloxy)-ethyl iodomethyl carbonate

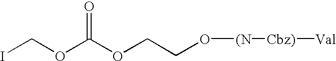

### a) 2-(N-CBz-L-valyloxy)-ethanol

To a mixture of DCC (11.4 g, 55 mmol), DMAP (0.611 g, 5 mmol) and ethyleneglycol (55.8 mL, 1 mol) in CH₂Cl₂ (300 mL) at 0° C., was added dropwise a solution of N-CBz-L-valine (12.6 g, 50 mmol) in CH₂Cl₂ (100 mL). After 1 h at 0° C., the temperature of the reaction mixture was allowed to assume room temperature and then the mixture was stirred for 5 h at room temperature. The mixture was filtered through a glass filter and the solvent was removed under reduced pressure. The crude product was column chromatographed (silica gel, 5→10% MeOH in CH₂Cl₂), to give 12.0 g 2-(N-CBz-L-valyloxy)-ethanol.

### b) 2-(N-CBz-L-valyloxy)-ethyl chloromethyl carbonate

To a mixture of 2-(N-CBz-L-valyloxy)-ethanol (12.0 g, 40.6 mmol) and pyridine (19.7 mL, 0.24 mmol) in CH₂Cl₂ (300 mL) at 0° C., was added dropwise chloromethyl chloroformate (10.5 g, 81.2 mmol). After 30 min at 0° C., the reaction mixture was washed with H₂O (200 mL). The H₂O phase was washed with CH₂Cl₂ (100 mL) and the solvent of the combined organic phases was removed under reduced pressure. The crude product was column chromatographed (silica gel, 0.5→1% MeOH in CH₂Cl₂), to give 8.26 g 2-(N-CBz-L-valyloxy)-ethyl chloromethyl carbonate.

### c) 2-(N-CBz-L-valyloxy)-ethyl iodomethyl carbonate

To a solution of 2-(N-CBz-L-valyloxy)-ethyl chloromethyl carbonate (3.88 g, 10 mmol) in acetonitrile (50 mL), was added sodium iodide (7.50 g, 50 mmol). The solution was stirred for 4 h at 60° C. The resulting suspension was filtered and the filtrate was evaporated. The residue was dissolved in CH₂Cl₂ and washed with aqueous sodium thiosulfate (0.1 M). The organic phase was dried (Na₂SO₄) and concentrated under reduced pressure, to give 4.51 g 2-(N-CBz-L-valyloxy)-ethyl iodomethyl carbonate.

¹H-NMR (CDCl₃): 7.34 (s, 5H), 5.93 (s, 2H), 5.26 (d, 1H), 5.11 (s, 2H), 4.48-4.26 (m, 5H) 2.28-2.10 (m, 1H), 0.97 (d, 3H), 0.90 (d, 3H).

### Example A-I-10

### 2,2-dimethyl-3-(N-CBz-D-valyloxy)-propionic acid iodomethyl ester

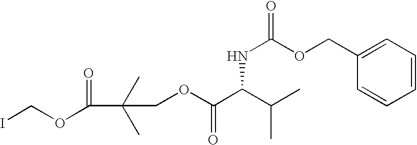

### a) 2,2-dimethyl-3-(N-CBz-D-valyloxy)-propionic acid

To a solution of 2,2-dimethyl propionic acid 4-methoxybenzyl ester (4.7 g, 20 mmole) and N-CBz-D-valine (5.5 g, 22 mmole) in dichloromethane (100 ml) were added 4-dimethyaminopyridine (305 mg, 2.5 mmole) and DCC (5.15 g, 25 mmole). After 18 hr, the solution was washed successively with sodium bicarbonate aqueous solution, citric acid solution and water. The organic phase was dried and the residue was dissolved in dichloromethane (100 ml). To the solution was added trifluoroacetic acid (10 ml). After 3 hr, it was evaporated and the product was isolated with silica gel column chromatography. 4.5 g.

¹H-NMR (CDCl₃): 7.36 (m, 5 H) 5.11 (s, 2H) 4.30 (m, 1H) 4.18 (dd, 2H) 2.17 (m, 1H), 1.23 (d, 6 H) 0.93 (m, 6H).

### b) 2,2-dimethyl-3-(N-CBz-D-Valyloxy )-propionic acid chloromethyl ester

(2,2-dimethyl-3-(N-CBz-D-valyloxy)-propionic acid (4.5 g, 12.8 mmole) was dissolved in dioxane (20 ml). To the solution was added tetrabutylammonium hydroxide aqueous solution (40%, 8.3 ml, 12.8 mmole). The solution was dried in vacuo, and it was coevaporated with toluene several times. The residue was dissolved in methylene chloride and then chloroiodomethane (18 ml, 260 mmole) was added to the solution. After 18 hr, the reaction solution was evaporated and the product was isolated with silica gel column chromatography. 3.5 g.

### c) 2,2-dimethyl-3-(N-CBz-D-valyloxy)-propionic acid iodomethyl ester

2,2-Dimethyl-3-(N-CBz-D-valyloxy)-propionic acid chloromethyl ester (2.4 g, 6 mmole) was dissolved in acetonitrile (30 ml). Sodium iodide (1.26 g, 8.4 mmole) was added to the solution. After reaction at 70° C. for 2 hr, the reaction mixture was filtered and the residue was dissolved in methylene chloride (20 ml) and refiltered. The solution was dried and gave the titled product. 2.68 g.

¹H-NMR (CDCl₃): 7.36 (m, 5 H) 5.90 (dd, 2H) 5.26 (d, 1H) 5.11 (s, 2H) 4.31 (m, 1H) 4.15 (dd, 2H) 2.18 (m, 1H) 1.22 (d, 6H) 0.92 (m, 6H).

### EXAMPLE A-I-11

### 4-(N-CBz-L-valyloxy) butyric acid Iodomethyl ester

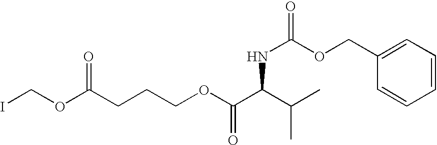

### a) 4-(N-CBz-L-valyloxy) butyric acid t-butyl ester

N-CBz-L-valine (16.25 g, 65 mmole) was dissolved in DMF (40 ml). To the solution was added potassium t-butoxide (7.24 g, 65 mmole). After 10 min, 4-bromobutyric acid t-butyl ester (12 g, 53 mmole) was added. The reaction mixture was kept at 65° C. for 2.5 hr and then poured into sodium bicarbonate aqueous solution and extracted with dichloromethane. The organic phase was dried and the product was isolated with silica gel column chromatography. 20.1 g.

### b) 4-(N-CBz-L-valyloxy)butyric acid chloromethyl ester

4-(N-CBz-L-valyloxy) butyric acid t-butyl ester (20 g, 50.8 mmole) was treated with trifluoroacetic acid (30 ml) at 0° C. for 3 h and then evaporated. The residue was coevaporated with toluene several time. The intermediate acid (2.56 g, 7.6 mmole) was dissolved in dioxane (10 ml) and to the solution was added tetrabutylammonium hydroxide (40%, 4.66 ml, 7.2 mmole). The solution was dried and dissolved in dichloromethane (20 ml) and then chloroiodomethane (10 ml, 144 mmole) was added to the solution. After 18 hr, the reaction solution was evaporated and the product was isolated with silica gel column chromatography. Yield 2.1 g.

### c) 4-(N-CBz-L-valyloxy)butyric acid iodomethyl ester

4-(N-CBz-L-valyloxy) butyric acid chloromethyl ester (1.54 g, 4 mmole) was dissolved in acetonitrile (15 ml). Sodium iodide (840 mg, 5.6 mmole) was added to the solution. After reaction at 55° C. for 3 hr, the reaction mixture was filtered and the residue was dissolved in methylene chloride (20 ml) and refiltered. The solution was dried and gave the titled product. Yield 1.9 g.

¹H-NMR (CDCl₃): 7.36 (m, 5H) 5.90 (dd, 2 H) 5.25 (d, 1H) 5.11 (s, 2H) 4.29 (dd, 1H 4.18 (t, 2H) 2.43 (t, 2H) 2.20 (m, 1H) 2.00 (m, 2H) 0.93 (dd, 6 H).

### EXAMPLE A-I-12

### Iodomethyl 3-(N-benzyloxycarbonyl-L-valyloxy)-benzoate

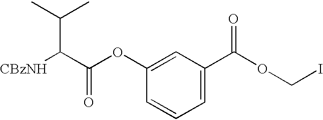

### a) 4-Methoxybenzyl 3-hydroxybenzoate

To a solution of 3-hydroxybenzoic acid (6.9 g, 50 mmole) in DMF (100 ml) was added potassium-tert.-butoxide (6.17 g, 55 mmole) and the mixture was stirred at room temperature for one hour. 4-Methoxybenzyl chloride (9.4 g, 60 mmole) was added and the mixture was stirred for 16 hours at 60° C. The mixture was evaporated under reduced pressure and ethyl acetate (250 ml) were added. The organic phase was washed five times with water, dried with sodium sulfate and evaporated under reduced pressure. The product was isolated by silica gel column chromatography with toluene/acetone. Yield: 10.5 g=81%

### b) 4-Methoxybenzyl 3-(N-benzyloxycarbonyl-L-valyloxy)benzoate

To a cooled solution of 4-methoxybenzyl 3-hydroxybenzoate (7.7 g, 29.8 mmole), 4-dimethylaminopyridine (0.73 g, 6 mmole) and N-benzyloxycarbonyl-L-valine (8.3 g, 33 mmole) in 100 ml dichloromethane was added dicyclohexyl-carbodiimide (7.22 g, 35 mmole) and the mixture was stirred for 2 days at room temperature. The mixture was cooled and the urethane was filtered. The solution was evaporated and ethyl acetate (250 ml) was added. The organic phase was washed twice with 5% acetic acid; 5% sodium hydrogencarbonate and water. The organic phase was dried with sodium sulfate and evaporated under reduced pressure. The product was isolated by silica gel column chromatography with hexane/ethyl acetate. Yield: 13.9 g=94%

### c) 3-(N-benzyloxycarbonyl-L-valyloxy)benzoic acid

To a solution of 4-methoxybenzyl-3-(N-benzyloxycarbonyl-L-valyloxy)-benzoate (13.7 g, 27.8 mmole) in dichloromethane (150 ml) was added trifluoroacetic acid (20 ml) and the mixture was stirred for 2 hours at room temperature. The solution was evaporated under reduced pressure and the product crystallized from toluene. Yield: 10.1 g=87%.

¹H-NMR (DMSO d-6) 1.01 (m, 6H) 2.21 (m, 1H) 4.17(d,d, 1H) 5.08 (s, 2H) 7.28-7.96 (m, 10H)

### d) Chloromethyl 3-(N-benzyloxycarbonyl-L-valyloxy)-benzoate

To a solution of 3-(N-benzyloxycarbonyl-valyloxy)benzoic acid (7.42 g, 20 mmole) in 1,4-dioxane (100 ml) was added a 40% solution of tetrabutylammonium hydroxide (12.97 g, 20 mmole) and the mixture was stirred 2 hours at room temperature. The mixture was evaporated under reduced pressure and co-evaporated two times with 1,4-dioxane and two times with toluene. The dried product was dissolved in dichloromethane (50 ml) and chloroiodomethane (35.3 g, 200 mmole) was added. The solution was stirred for two days at room temperature and evaporated under reduced pressure. Ethyl acetate (100 ml) was added and the organic phase washed twice with water, dried with sodium sulfate and evaporated under reduced pressure: The product was isolated by silica gel column chromatography. Yield: 3.8 g=45%.

### e) Iodomethyl 3-(N-benzyloxycarbonyl-L-valyloxy)-benzoate

To a solution of chloromethyl 3-(N-benzyloxycarbonyl-L-valyloxy)-benzoate (2.0 g, 4.76 mmole) in dry acetone (30 ml) was added sodium iodide (3.15 g, 21 mmole) and the mixture was stirred overnight at room temperature. The mixture was evaporated under reduced pressure and extracted with ethyl actate/water. The organic phase was washed with a 5% sodium thiosulfate solution, dried with sodium sulfate and evaporated under reduced pressure. Yield: 2.3 g=94%.

¹H-NMR (CDCl₃) 1.02 (m, 6H) 2.38 (m, 1H) 4.56 (d, d , 1H) 5.14 (s , 2H) 5.30 (d, 1H) 6.14 (s, 2H) 7.26-7.50 (m, 7H) 7.80(s, 1H) 7.96 (d, 1H)

### EXAMPLE A-I-13

### Iodomethyl 3-(N-benzyloxycarbonyl-L-valyloxy)-propionate

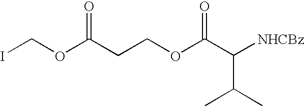

### a) 3-buten-1-yl-3-(N-benzyloxycarbonyl)-propionate

To a solution of 3-buten-1-ol (2.16 g, 30 mmole), N-benzyloxycarbonyl-1-valine (8.29 g, 33 mmole) and 4-dimethylaminopyridine (0.37 g, 3 mmole) in dichloromethane (80 ml) was added dicyclohexyl-carbodiimide (7.22 g, 35 mmole) and the mixture was stirred overnight at room temperature. The mixture was cooled and the urethane was filtered. The solution was evaporated under reduced pressure and ethyl acetate (200 ml) was added. The organic phase was washed twice with 5% acetic acid, 5% sodium hydrogencarbonate and water. The organic phase was dried with sodium sulfate and evaporated under reduced pressure. The product was isolated by silica gel column chromatography with hexane/ethyl acetate. Yield: 8.3 g=90%.

### b) 3-(N-benzyloxycarbonyl-L-valyloxy)-propanoic acid

To a solution of 3-buten-1-yl -3-(N-benzyloxycarbonyl-L-valyloxy)-propionate (9.2 g, 30 mmole) in 150 ml benzene was added tetrabutylammonium bromide (1.62 g, 5 mmole) and 100 ml water. The mixture was cooled to about 5° C. and potassium permanganate (14.82 g, 90 mmole) was added in portions. The mixture was stirred 2 hours at room temperature, diluted with water and decolorizedby the addition of sodium bisulfite. The mixture was acidified with 2M hydrogen chloride and extracted 3 times with ethyl acetate. The combined organic phases were washed with water and dried with sodium sulfate. The solution was evaporated under reduced pressure and the product isolated by silica gel column chromatography with hexane/ethyl acetate. Yield: 5.4 g=55%.

¹H-NMR (DMSO d-6) 0.90 (m, 6H) 2.5 (m, 2H) 3.88 (d, d, 1H) 4.32 (m, 2H) 5.03 (s, 2H) 7.36 (m, 5H) 7.68 (d, 1H)

### c) Chloromethyl 3-(N-benzyloxycarbonyl-L-valyloxy)-propionate

To a solution of 3-(N-benzyloxycarbonyl-L-valyloxy)propanoic acid (5.2 g, 16.08 mmole) in 1,4-dioxane (50 ml) was added a 40% solution of tetrabutylammonium hydroxide (10.43 g, 16.08 mmole) and the mixture was stirred 2 hours at room temperature. The mixture was evaporated under reduced pressure and co-evaporated two times with 1,4-dioxane and two times with toluene. The dried product was dissolved in 40 ml dichloromethane and chloroiodomethane (28.4 g. 160 mmole) was added. The solution was stirred for two days at room temperature and evaporated under reduced pressure. Ethyl acetate (100 ml) was added and the organic phase washed twice with water, dried with sodium sulfate and evaporated under reduced pressure. The product was isolated by silica gel column chromatography. Yield: 2.2 g=35%

### d) Iodomethyl3-(N-benzyloxycarbonyl-L-valyloxy)-propionate

To a solution of chloromethyl 3-(N-benzyloxycarbonyl-L-valyloxy)-propionate (2.05 g, 5.51 mmole) in dry acetone (50 ml) was added sodium iodide (4.12 g, 27.5 mmole) and the mixture was stirred overnight at room temperature. The mixture was evaporated under reduced pressure and extracted with ethyl acetate water. The organic phase was washed with a 5% sodium thiosulfate solution, dried with sodium sulfate and evaporated under reduced pressure. Yield: 2.35 g=92%.

¹H-NMR (CDCl₃) 0.94 (m, 6H) 2.17 (m, 1H) 2.68 (t, 2H) 4.40 (m, 3H) 5.12 (s, 2H) 5.91 (s, 2H) 7.26 (m, 5H).

### EXAMPLE A-I-15

### 1-(N-benzyloxycarbonyl-L-valyloxy)-2-methyl-2-propyl iodomethyl carbonate

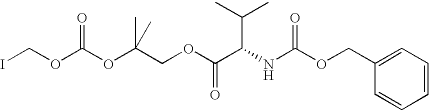

### (a) 1-(N-benzyloxycarbonyl-L-valyloxy)-2-methyl-2-propanol

N-Benzyloxycarbonyl-L-valine (2.02 g, 8.0 mmol), 4-dimethylaminopyridine (100 mg, 0.8 mmol), and ), and dicyclohexylcarbodiimide (2.04 g, 9.9 mmol, in 20 mL CH₂Cl₂) were added to 2-methyl-1,2-propanediol (12.2 mmol) in 30 mL dry CH₂Cl₂, with cooling in an ice bath. DMF (5 mL) was added. After stirring for 5 h at 10° C., the reaction mixture was filtered, concentrated, and then redissolved in ethyl acetate. The organic solution was washed with saturated NaCl, dried over anhydrous Na₂SO₄, and concentrated. Flash column chromatography (silica, 2/1 petroleum ether—ethyl acetate) gave 2.3 g of the title compound.

### (b) 1-(N-benzyloxycarbonyl-L-valyloxy)-2-methyl-2-propyl chloromethyl carbonate

All of the alcohol from above was dissolved in 35 mL dry CH₂Cl₂ and cooled in an ice bath. Pyridine (3.50 mL, 43.4 mmol) was added, followed by chloromethyl chloroformate (1.30 mL, 14.4 mmol). After 1 h, the ice bath was removed and stirring was continued for 2 h at ambient temperature. The mixture was diluted with CH₂Cl₂ (50 mL) and washed with water (50 mL), and then brine (2×25 mL). Drying over anhydrous Na₂SO₄ of the combined organic phases and concentration under vacuum, coevaporating several times with toluene, gave a yellow-brown oil that was subjected to flash column chromatography (silica, 2/1 petroleum ether—ethyl acetate) to yield 2.86 g (86% from N-benzyloxycarbonyl-L-valine) of the title compound.

### c) 1-(N-benzyloxycarbonyl-L-valyloxy)-2-methyl-2-propyl iodomethyl carbonate

A mixture of the chloride (2.84 g, 6.84 mmol) from step (b) and NaI (4.15 g, 27.2 mmol) in 68 mL dry acetonitrile was refluxed at 75° C. for 4 h. After evaporation of solvent under vacuum, the residue was partitioned between ethyl acetate (80 mL) and water (40 mL), and the organic layer was washed with 5% Na₂S₂O₃ (15 mL) and brine (25 mL). Drying the organic phase over anhydrous Na₂SO₄ and concentration gave a yellow oil that was subjected to flash column chromatography (silica, 2/1 petroleum ether—ethyl acetate) to furnish 3.29 g (95%) of the title compound.

¹H NMR (250 MHz, CDCl₃) 0.90and 0.94 (2d, 3H each, J=6.8 Hz), 1.52 (s, 6H), 2.17 (m, 1H), 4.35 (m, 1H), 4.22 and 4.39 (ABq, 2H, J_{AB}=11.7 Hz), 5.10 (s, 2H), 5.30 (d, 1H), 5.86 (s, 2H), 7.34 (s, 5H)

### EXAMPLE A-I-16

### Iodomethyl 3,4-di-(N-CBZ-L-valyloxy)hydrocinnamate

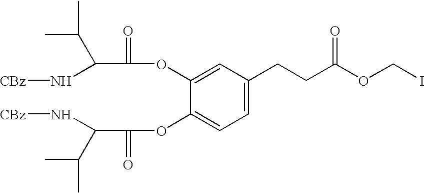

### a) 4-Methoxybenzyl-3,4-dihydroxyhydrocinnamate

3,4-Dihydroxycinnamic acid (6.5 g, 35.7 mmol) was dissolved in DMF (50 ml) and cooled to 0° C. on an ice-bath. 4-Potassium tert-butoxide (35.7 mmol), was then added and the mixture was left for approximately 30 min at 0° C., followed by dropwise adition of 4-methoxy-benzylchloride (39 mmol) in DMF (25 ml). The mixture was allowed to reach room temperature and left over-night. The solvent was then evaporated and the crude product was purified by chromatography (ethyl acetate-hexane, 1:1) to give 6 g of the title compound (55%).

### b) 4-Methoxybenzyl-3,4-di-(N-CBZ-L-valyloxy)hydrocinnamate

4-Methoxybenzyl-3,4-dihydroxyhydrocinnamate (5 g, 16.5 mmol), N,N-dimethylaminopyridine (2 g, 16.5 mmol), N,N′-dicychlohexyl carbodiimide (8.5 g, 41.3 mmol) and Cbz-L-valine (10.4 g, 41.3 mmol) were dissolved in dichloromethane (50 ml). After 4 h, the the mixture was filtered and evaporated onto silica gel and purified by chromatography (hexane-EtOAc, 5:2→3:2) to give pure title product (10.1 g, 79%).

### c) 3,4-Di-(N-CBZ-L-valyloxy)hydrocinnamic acid

4-Methoxybenzyl-3,4-di-(N-CBZ-L-valyloxy)hydrocinnamate (10 g, 13 mmol) was dissolved in dichloromethane and 1,1,1 trifluoroacetic acid (30 ml) and left at ambient temperature for 3.5 h. Evaporation under reduced pressure and purification by chromatography (chloroform-methanol, 10:1) yielded 6.7 g (80%) pure title product.

¹H NMR (CDCl₃45° C.): 7.24-7.0 (m, 13H), 5.65 (br s, 1H), 5.55 (br s, 1H), 5.1 (m, 4H), 4.46 (m, 2H), 2.95 (t, 2H), 2.66 (t, 2H), 2.35 (m, 2H).

### d) Chloromethyl 3,4-di-(N-CBZ-L-valyloxy)hydrocinnamate

3,4-Di-(N-CBZ-L-valyloxy)hydrocinnamic acid (4.2 g, 6.47 mmol) was dissolved in dioxane (70 ml). Tetrabutylammonium hydroxide was added dropwise until pH=8. The solvent was then removed under reduced pressure The solid was redissolved in dioxane (30 ml) and toluene (30 ml) and evaporated. The procedure was repeated twice (for removal of water). Dichloromethane (60 ml) and chloro-iodomethane was added in one portion and the mixture was left at ambient temperature for 6 h. Evaporation of the solvent and purification by chromatography yielded 1.7 g title product (38%).

### e) Iodomethyl 3,4-di-(N-CBZ-L-valyloxy)hydrocinnamate

Chloromethyl 3,4-di-(N-CBZ-L-valyloxy)hydrocinnamate (1.9 g, 2.7 mmol) and sodium iodide (2 g, 13.3 mmol) were dissolved in acetonitrile (50 ml) and heated to 65° C. for 60 min. The solvent was removed under reduced pressure and the residue was taken up in dichloromethane and filtrated. Removal of the solvent and purification by chromatography (ethyl acetate-hexane, 2:5) gave pure title product (1.9 g, 90%).

¹H NMR (CDCl₃45° C.): 7.34-7.02 (m, 13H), 5.89 (s, 2H), 5.64 (br s, 2H), 5.14-5.02 (m, 4H), 4.47 (m, 2H), 2.96 (t, 2H), 2.64 (t, 2H), 2.33 (m, 2H), 1.08-0.99 (m, 12H)

### EXAMPLE A-I-17

### 3-(N-CBZ-L-valyloxy)phenyl iodomethyl carbonate

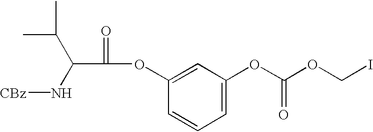

### a) 3-(N-CBz-L-valyloxy)phenol

CBz-L-valine (10 g, 40 mmol), 1,3-dihydroxybenzene (8.7 g, 79 mmol) N,N′dicychlohexylcarbodiimide (10.2 g, 44 mmol) and 4-dimethylaminopyridine (2.4 g, 20 mmol) were dissolved in DMF (50 ml) and left at ambient temperature overnight. The reaction mixture was filtered, the solvent removed under reduced pressure and the crude product was taken up in dichloromethane and filtered. Removal of the solvent followed by purification by chromatography (chloroform-methanol, 10:1) yielded pure title product (10.9 g, 79%).

### b) (N-CBZ-L-valyloxy)phenyl chloromethyl carbonate

3-(N-CBz-L-valyloxy)phenol (5.4 g, 15 7 mmol) was dissolved in dichloromethane (70 ml) and cooled in an ice-bath. Pyridine (1.2 g, 23.5 mmol was added followed by dropwise addition of 1-chloro-methylchloroformate (2.3 g, 18.8 mmol) in dichloromethane (10 ml). The mixture was left at room temperature for 4 h. Water (25 ml) was then added and the phases were separated. The organic layer was washed with 0.01 M aqueous hydrochloric acid (25 ml). Purification by chromatography (ethyl acetate-hexane, 1:1) gave the title compound (4.5 g, 65%)

### c) 3-(N-CBZ-L-valyloxy)phenyl iodomethyl carbonate

(N-CBZ-L-valyloxy)phenyl chloromethyl carbonate (1.5 g, 3.44 mmol) and sodium iodide (2 g, 13.3 mmol) were stirred at 60° C. in acetonitrile (50 ml) for 4.5 h. The mixture was filered, the solvent removed and the crude product was taken up in 100 ml hexane-ethyl acetate, 1:1, and filtered through a sintered glass funnel, packed with 2 cm silica gel. Removal of the solvent yielded pure title product (1.68 g, 92%)

¹H NMR (CDCl₃45° C.): 7.38-7.02 (m, 9H), 6.03 (s, 2H), 5.2 (br s, 1H), 5.14 (s, 2H), 4.48 (m, 1H), 2.30 (m, 1H), 1.09-1.01 (m, 6H)

### EXAMPLE A-I-18

### Iodomethyl 2-(N-CBZ-L-valyloxy)phenylacetate

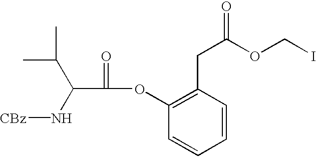

### a) 4-Methoxybenzyl 2-hydroxyphenylacetate

2-hydroxyphenylacetic acid (10 g, 66 mmol) was dissolved in N,N-dimethyl-formamide (100 ml) and cooled on ice-bath. Potassium tert-butoxide (8.85 g, 78 mmol) was added. The mixture was left for 30 min and allowed to reach room temperature. 4-Methoxy-benzylchloride (11.7 g, 72 mmol) in N,N-dimethyl-formamide (30 ml) was then added dropwise, under nitrogen atmosphere and left over-night. The solvent was evaporated under reduced pressure and the crude mixture was dissolved in ether (100 ml) and washed with water (25 ml), brine and dried over sodium sulphate. Chromatography (hexane-ethyl acetate, 2:1) followed by recrystallization (hexane-ethyl acetate) gave the title compound (7.6 g, 42%).

### b) 4-Methoxybenzyl 2-(N-CBz-L-valyloxy)phenylacetate

4-Methoxybenzyl 2-hydroxyphenylacetate 3 g, 11 mmol), N,N,-dichyclohexyl-carbodiimide (2.7 g, 13.2 mmol), dimethylaminopyridine (0.134 g, 1.1 mmol) and CBz-L-valine (3.3 g, 13.2 mmol) were dissolved in dichloromethane (50 ml). After the weekend the solid was filtered off, the solvent removed under reduced pressure and the crude product purified by chromatography (ethyl acetate, hexane, 1:2) to give the title compound (5.2 g, 93%).

### c) 2-(N-CBz-L-valyloxy)phenylacetic acid

4-Methoxybenzyl 2-(N-CBz-L-valyloxy)phenylacetate (4.25 g, 8.4 mmol), was dissolved in dichloromethane (40 ml). Triflouroacetic acid (8 ml) was added with cooling on ice. The mixture was allowed to reach room temperature and stirred for 40 min. The solvent was removed under reduced pressure and the crude product was recrystallized twice (hexan-ethyl acetate+a small amount of dichloromethane) to give the title compound (2.6 g, 80%).

¹H NMR (CDCl₃45° C.): 7.35-7.08 (m, 9H), 5.35 (br s, 1H), 5.13 (s, 2H), 4.48 (m, 1H), 3.57 (s, 2H), 2.33 (m, 1H), 1.08 (d, 3H), 1.02 (d, 3H).

### d) Chloromethyl 2-(N-CBZ-L-valyloxy)phenylacetate

This compound was prepared in poor yield from 2-(N-CBz-L-valyloxy)phenylacetic acid (5.5 g, 14.3 mmol) by an unoptimized procedure essentially as described in Example A-I-16 d). Yield: 0.265 g

¹H NMR (CDCl₃45° C.): 7.28-7.01 (m, 9H), 5.55 (s, 2H), 5.2 (br s, 1H), 5.07 (s, 2H), 4.43 (m, 1H), 3.53 (s, 2H), 2.26 (m, 1 H), 1.02 (d, 3H), 0.95 (d, 3H).

### e) Iodomethyl 2-(N-CBZ-L-valyloxy)phenylacetate

Chloromethyl 2-(N-CBZ-L-valyloxy)phenylacetate is treated with Nal and purified as described in the Examples above to yield the title compound.

### EXAMPLE A-I-19

### Iodomethyl 4-(N-CBZ-L-valyloxyxy)phenylacetate

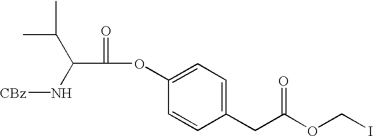

### a) 4-Methoxybenzyl 4-hydroxyphenylacetate

Prepared from 4-hydroxyphenylacetic acid (10 g, 65.7 mmol) in 70% yield by the same procedure as for Example A-I-18a) above, but wherein the solvent for the recrystallization was changed to hexane-ether.

### b) 4-Methoxybenzyl 4-(N-CBz-L-valyloxy)phenylacetate

Prepared from 4-methoxybenzyl 4-hydroxyphenylacetate (3 g, 11 mmol) by the same procedure as for Example A-I-18b) in 87% yield. Solvent for chromatography: ethyl acetate-hexane, 1:2.

### c) 4-(N-CBZ-L-valyloxy)phenylacetic acid

Prepared in 82% yield from 4-methoxybenzyl 4-(N-CBz-L-valyloxy)phenylacetate (1.6 g, 288 mmol) by the procedure described for Example A-I-18c). Solvent for recrystallization: hexane-ether and a small amount of dichloromethane.

¹H NMR (CDCl₃45° C.): 7.36-7.27 (m, 7H), 7.02 (d, 2H), 5.25 (d, 1H), 5.14 (s, 2H), 4.52 (m, 1H), 3.64 (s, 2H), 2.3 (m, 1H), 1.08 (d, 3H), 1.02 (d, 3H).

### d) Chloromethyl 4-(N-CBZ-L-valyloxy)phenylacetate

Prepared from 4-(N-CBZ-L-valyloxy)phenylacetic acid (3 g, 7.8 mmol) in 26% yield by the same procedure as described for Example A-I-18d). Solvent for chromatography: hexane-ether, 3:2.

### e) Iodomethyl 4-(N-CBZ-L-valyloxy)phenylacetate

Chloromethyl 4-(N-CBZ-L-valyloxy)phenylacetate (0.83 g, 1.9 mmol) and sodium iodide (1.15 g, 7.6 mmol) were heated in acetonitril (45 ml) for 5 h. The mixture was filtrated, the solvent removed, taken up in dichloromethane and filtrated again. Evaporation and purification by chromatography (ether-hexane, 2:3) yielded the title product (0.8 g, 80%).

¹H NMR (CDCl₃45° C.): 7.38-7.09 (m, 4H), 5.84 (s, 1H), 5.30 (brs, 1H), 5.15 (s, 2H), 4.5 (m, 1H), 3.56 (s, 2H), 2.36 (m, 1H), 1.10 (d, 3H), 1.00 (d, 3H).

### EXAMPLE A-I-20

### Iodomethyl 4-(2-N-benzyloxycarbonyl-L-valyloxyethyl)benzoate

### a) 4-(2-N-benzyloxycarbonyl-L-valyloxyethyl)-toluene

To a cooled solution of 4-methylphenylethanol-2 (5.0 g, 36.7 mmole), 4-dimethylaminopyridine (0.98 g, 8 mmole) and N-benzyloxycarbonyl-L-valine (10.05 g, 40 mmole) in dichloromethane (120 ml) was added dicyclohexyl-carbodiimide (9.1 g, 44 mmole) and the mixture was stirred overnight at room temperature. The mixture was cooled and the urethane was filtered. The solution was evaporated under reduced pressure and ethyl acetate (250 ml) was added. The organic phase was washed twice with 5% acetic acid, 5% sodium hydrogencarbonate and water. The organic phase was dried with sodium sulfate and evaporated under reduced pressure. The product was isolated by silica gel column chromatography

### b) 4-(2-N-benzyloxycarbonyl-L-valyloxyethyl)-benzoic acid

To a cooled mixture of chromic anhydride (7.55 g, 75 mmole) in acetic acid (100 ml) was added dropwise a solution of 4-(2-N-benzyloxycarbonyl-L-valyloxyethyl)-toluene (9.3 g, 25.1 mmole) in acetone (50 ml). The mixture was stirred at room temperature for 3 days and reduced to about 100 ml. 600ml 10% sodium chloride solution was added and the mixture was extracted four times with ethyl acetate. The organic phase was washed with brine and dried with sodium sulfate. The solutionwas evaporated under reduced pressure and the product was islolated by silica gel column chromatography with dichloromethane/methanol. Yield 2,1 g 21%.

¹H-NMR (CDCl₃) 0.79 (d, 3H) 0.90 (d, 3H) 2.08 (m,1H) 3.04 (t, 2H) 4.28 d, 1H)4.39 (m, 2H) 5.11 (s, 2H) 5.26 (d, 1H) 7.34 (m, 7H) 8.04 (d, 2H)

### c) Chloromethyl 4-(2-N-benzyloxycarbonyl-L-valyloxyethyl)benzoate

To a solution of 4-(2-N-benzyloxycarbonyl-L-valyloxyethyl)benzoic acid (2.0 g, 5.0 mmole) in 1,4-dioxane (20 ml)was added a 40% solution of tetrabutylammonium hydroxide (3.1 g, 4.75 mmole) and the mixture was stirred 2 hours at room temperature. The mixture was evaporated under reduced pressure and coevaporated two times with 1,4-dioxane and two times with toluene. The dried product was dissolved in dichloromethane (10 ml) and iodochloromethane (13.2 g, 75 mmole) was added The solution was stirred overnight at room temperature and evaporated under reduced pressure. About 50 ml ethyl acetate were added and the organic phase washed twice with water, dried with sodium sulfate and evaporated under reduced pressure. The product was isolated by silica gel column chromatography.Yield: 0.5 g=23%

### d) Iodomethyl 4-(2-N-benzyloxycarbonyl-L-valyloxyethyl)benzoate

To a solution of chloromethyl 4-(2-N-benzyloxycarbonyl-L-valyloxyethyl)benzoate (0.5 g, 1.11 mmole). In dry acetone (10 ml) was added sodium iodide (0.75 g, 5.0 mmole) and the mixture was stirred overnight at room temperature. The mixture was evaporated under reduced pressure and extracted with ethyl actate/water. The organic phase was washed with a 5% sodium thiosulfate solution, dried with sodium sulfate and evaporated under reduced pressure.Yield: 0.53 g=88%.

¹H-NMR (CDCl₃) 0.88 (d, 3H) 0.90 (d, 3H) 2.08 (m, 1H) 3.02 (t, 2H) 4.28 (d, d, 1H) 4.38 (m, 2H) 5.10 (s, 2H) 5.22 (d, 1H) 6.15 (s, 2H) 7.35(m, 7H) 7.98 (d, 2H )

### EXAMPLE A-I-21

### Iodomethyl 2-(N-benzyloxycarbonyl-L-isoleucyloxymethyl) 2-methyl propionate

### a) 4-methoxybenzyl 2-(N-benzyloxycarbonyl-L-isoleucyloxymethyl) -2-methyl propionate

To a cooled solution of 4-methoxybenzyl 2-(hydroxymethyl)-2-methyl propionate (6.0 g, 25 mmole), 4-dimethylaminopyridine (0.61 g, 5 mmole) and N-benzyloxycarbonyl-L-isoleucine (6.90 g, 26 mmole) in dichloromethane (100 ml) was added dicyclohexyl-carbodiimide (6.2 g, 30 mmole) and the mixture was stirred overnight at room temperature.The mixture was cooled and the urethane was filtered. The solution was evaporated and 200 ml ethyl acetate was added, The organic phase was washed twice with 5% acetic acid, 5% sodium hydrogencarbonate and water. The organic phase was dried with sodium sulfate and evaporated under reduced pressure. The product was isolated by silica gel column chromatography with toluene/acetone. Yield: 11.7 g=96%.

### 2-(N-benzyloxycarbonyl-L-isoleucyloxymethyl)-2-methyl)propionic acid

To a solution of 4-methoxybenzyl 2-(N-benzyloxycarbonyl-L-isoleucyloxymethyl)-2-methyl propionate (11.0 g, 22.6 mmole) in 100 ml dichloromethane was added trifluoroacetic acid (15 ml) and the mixture was stirred overnight at room temperature. The solution was evaporated under reduced pressure and coevaporated two times with toluene. The residue was stirred 1 hour with 100 ml ethanol and the white solid was filtered (byproduct). The solution was evaporated under reduced pressure and the product was isolated by silica gel column chromatography with hexane/ethyl acetate. Yield: 7.4 g=89%.

¹H-NMR (CDCl₃) 0.90 (m, 6H) 1.26 (m, 8H) 1.88 (m, 1H) 4.12 (d, d, 2H) 4.38 (d, d, 1H) 5.10 (s, 2H) 5.32 (d, 1H) 7.28 (m, 5H)

### c) Chloromethyl 2-(N-benzyloxycarbonyl-L-isoleucyloxy)-2-methyl propionate

To a solution of 2-(N-benzyloxycarbony-L-isoleucyloxymethyl)-2-methyl propionic acid (7.0 g, 19 mmole) in 80 ml 1,4-dioxane was added a 40% solution of tetrabutylammonium hydroxide (12.4 g, 19 mmole) and the mixture was stirred 2 hours at room temperature. The mixture was evaporated under reduced pressure and co-evaporated two times with 1,4-dioxane and two times with toluene. The dried product was dissolved in 25 ml dichloromethane and iodochloromethane (33.7 g, 190 mmole) was added. The solution was stirred overnight at room temperature and evaporated under reduced pressure. About 100 ml ethyl actate was added and the organic phase washed twice with water, dried with sodium sulfate and evaporated under reduced pressure. The product was isolated by silica gel column chromatography with toluene/acetone. Yield: 4.2=54%

### d) Iodomethyl 2-(N-benzyloxycarbonyl-L-isoleucyloxymethyl)-2-methyl propionate

To a solution of chloromethyl 2-(N-benzyloxycarbonyl-L-isoleucyloxymethyl)-2-methyl propionate (3.0 g, 7.2 mmole) in 50 ml dry acetone was added sodium iodide (4.8 g, 32 mmole) and the mixture was stirred overnight at room temperature. The mixture was evaporated under reduced pressure and extracted with ethyl actate water. The organic phase was washed with a 5% sodium thiosulfate solution, dried with sodium sulfate and evaporated under reduced pressure. Yield: 3.3 g=90%.

¹H-NMR (CDCl₃) 0.93 (m, 6H) 1.23 (m, 8H) 4.12 (m, 2H) 4.38 (d, d, 1H) 5.10 (s, 2H) 5.26 (d, 1H) 5.92 (m, 2H) 5.35 (m, 5H)

### EXAMPLE A-I-22

### Iodomethyl 4-(N-benzyloxycarbonyl-L-valyloxy)cyclohexanoate

### a) 4-Methoxybenzyl 4-hydroxycyclohexanoate

To a solution of ethyl 4-hydroxycyclohexanoate (8.61 g, 50 mmole) in 50 ml ethanol was added a solution of potassium hydroxide 85% (3.63 g, 55 mmole) and the mixture was stirred for 6 hours at 70° C. The mixture was evaporated under reduced pressure, coevaporated two times with N,N-dimethylformamide and reduced to about 100 ml. 4-Methoxybenzyl chloride (9.4 g, 60 mmole) was added and the mixture was stirred for 18 hours at 60° C. The mixture was evaporated under reduced pressure and 250 ml ethyl acetate was added. The organic phase was washed five times with water, dried with sodiun sulfate and evaporated under reduced pressure. Yield: 13.2 g=100% (crude)

### b) 4-methoxybenzyl 4-(N-benzyloxycarbonyl-L-valyloxy)-cyclohexanoate

To a cooled solution of 4-methoxybenzyl 4-hydroxycyclohexanoate (7.5 g, 28 mmole), 4-dimethylaminopyridine (0.73 g, 6 mmole) and N-benzyloxycarbonyl-L-valine (7.54 g, 30 mmole) in dichloromethane (90 ml) was added dicylohexyl-carbodiimide (6.8 g, 33 mmole) and the mixture was stirred for 2 days at room temperature. The mixture was cooled and the urethane was filtered. The solution was evaporated and 250 ml ethyl acetate was added.The organic phase was washed twice with 5% acetic acid, 5% sodium hydrogencarbonate and water. The organic phase was dried with sodium sulfate and evaporated under reduced pressure. The product was isolated by silica gel column chromatography with toluene/acetone. Yield: 13 g=93%

### c) 4-(N-benzyloxycarbonyl-L-valyloxy) cyclohexanoic acid

To a solution of 4-methoxybenzyl 4-(N-benzyloxycarbonyl-L-valyloxy)-cyclohexanoate (12 g, 24.1 mmole) in dichloromethane (100 ml) was added trifluoroacetic acid (20 ml) and the mixture was stirred for 3 hours at room temperature. The solution was evaporated under reduced pressure and coevaporated two times with toluene. The residue was stirred 1 hour with about 100 ml ethanol and the white solid was filtered (byproduct). The solution was evaporated under reduced pressure and the product was isolated by silica gel column chromatography with toluene/acetone. Yield: 6.8 g=74%.

¹H-NMR (CDCl₃) 0.91 (m, 6H) 1.52-2.54 (m, 10H) 4.28 (m, 1H) 4.82-5.08 (m, 1H) 5.11 (s, 2H) 5.28 (d, 1H) 7.36 (m, 5H)

### d) Chloromethyl 4-(N-benzyloxycarbonyl-L-valyloxy)-cyclohexanoate

To a solution of 4-(N-benzyloxycarbonyl-L-valyloxy) cyclohexanoic acid (6.6 g, 20 mmole) in 1,4-dioxane (70 ml) was added a 40% solution of tetrabutylammonium hydroxide (11.34 g, 17.5 mmole) and the mixture was stirred 2 hours at room temperature. The mixture was evaporated under reduced pressure and co-evaporated two times with 1,4-dioxane and two times with toluene. The dried product was dissolved in 60 ml dichloromethane and iodochloromethane (30.9 g, 175 mmole) was added. The solution was stirred for two days at room temperature and evaporated under reduced pressure. About 100 ml ethyl actate was added and the organic phase washed twice with water, dried with sodium sulfate and evaporated under reduced pressure. The product was isolated by silica gel column chromatography with toluene/acetone. Yield: 4.1 g=55%.

### e) Iodomethyl 4-(N-benzyloxycarbonyl-L-valyloxy)-cyclohexanoate

To a solution of chloromethyl 4-(N-benzyloxycarbonyl-L-valyloxy)-cyclohexanoate (4.0 g, 9.4 mmole) in dry acetone (50 ml) was added sodium iodide (6.3 g, 42 mmole) and the mixture was stirred overnight at room temperature. The mixture was evaporated under reduced pressure and extracted with ethyl actate water. The organic phase was washed with a 5% sodium thiosulfate solution, dried with sodium sulfate and evaporated under reduced pressure. Yield 4.5 g=93%.

¹H-NMR (CDCl₃) 0.90 (m, 6H) 1.52-2.02 (m, 8H) 2.18 (m, 1H) 2.43 (m, 1H) 4.30 (m, 1H) 4.76-5.08 (m, 1H) 5.11 (s, 2H) 5.26 (d, 1H) 5.91 (d, 2H) 7.34 (m, 5H)

### EXAMPLE A-I-23

### Iodomethyl 2-(N-benzyloxycarbonyl-L-valyloxymethyl)-2-ethyl butyrate

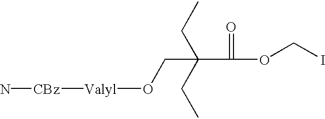

### a) 2-(N-benzyloxycarbonyl-L-valyloxymethyl)-2-ethyl butan-1-ol

To a cooled solution of 2-ethyl-2-hydroxymethyl-butan-1-ol (33.19, 250 mmole), 4-dimethylaminopyridine (1.22 g, 10 mmole) and N-benzyloxycarbonyl-L-valine (12.6 g, 50 mmole) in 350 ml dichloromethane was added dropwise a solution of dicyclohexyl-carbodiimide (12.49, 60 mmole) in 50 ml dichloromethane. The mixture was stirred 2 days at room temperature and cooled. The urethane was filtered and the solution evaporated under reduced pressure. 350 ml ethyl acetate was added and the organic phase was washed twice with 5% acetic acid, 5% sodium-hydrogencarbonate and water. The organic phase was dried with sodium sulfat and evaporated under reduced pressure. The product was isolated by silica gel column chromatography with dichloromethane/methanol. Yield: 16.4 g=90%.

### c) 2-(N-benzyloxycarbonyl-L-valyloxymethyl )-2-ethyl-butyric acid

To a cooled mixture of chromic anhydride (8.5 g, 85,2 mmole) in 100 ml acetic acid was added dropwise a solution of 2-(N-benzyloxycarbonyl-L-valyoxymethyl)-2-ethyl-butan-1-ol (10.4 g, 28.4 mmole) in 50 ml acetone and the mixture was stirred 24 hours at room temperature. The mixture was added to 1000 ml 10% sodium chloride solution and extracted four times with ethyl acetate. The organic phase was washed twice with brine, dried with sodium sulfate and evaporated under reduced pressure. The product was isolated by silica gel column chromatography with hexane/ethyl acetate. Yield: 7 g=65%.

¹H-NMR (CDCl₃) 0.88 (m, 12H) 1.67 (m, 4H) 2.14 (m, 1H) 4.26 (m, 3H) 5.10 (s, 2H) 5.30 (d, 2H) 7.34 (m, 5H)

### d) Chloromethyl 2-(N-benzyoxycarbonyl-L-valyloxymethyl -2-ethyl butyrate

To a solution of 2-(N-benzyloxycarbony-L-valyloxymethyl)-2-ethyl-butyric acid (7.2 g,18.9 mmole) in 1,4-dioxane (80 ml) was added a 40% solution of tetrabutylammonium hydroxide (12.26 g, 18.9 mmole) and the mixture was stirred 2 hours at room temperature. The mixture was evaporated under reduced pressure and co-evaporated once with 1,4-dioxane and two times with toluene. The dried product was dissolved in 30 ml dichloromethane and iodochloromethane (49.4 g, 280 mmole) was added. The solution was stirred for two days at room temperature and evaporated under reduced pressure. About 100 ml ethyl actate were added and the organic phase washed twice with water, dried with sodium sulfate and evaporated under reduced pressure. The product was isolated by silica gel column chromatography. Yield: 5.2 g=63%

### e) Iodomethyl 2-(N-benzyloxycarbonyl-L-valyloxymethyl)-2-ethyl butyrate

To a solution of chloromethyl 2-(N-benzyloxycarbonyl-L-valyloxymethyl)-2-ethyl butyrate (5.0 g, 11.7 mmole) in dry acetone (60 ml) was added sodium iodide (7.5 g, 50 mmole) and the mixture was stirred overnight at room temperature. The mixture was evaporated under reduced pressure and extracted with ethyl actate water. The organic phase was washed with a 5% sodium thiosulfate solution, dried with sodium sulfate and evaporated under reduced pressure. Yield: 5.4 g=90%.

¹H-NMR (CDCl₃) 0.92 (m, 12H) 1.65 (m, 4H) 2.18 (m, 1H) 4.28 (m, 3H) 5.10 (s, 2H) 5.22 (d, 1H) 5.92 (s, 2H) 7.36 (m, 5H)

### EXAMPLE A-I-24

### 2-(N-(iodomethoxycarbonyl)-amino)-2-methyl-1-(N-benzyloxycarbonyl-L-valyloxy)-propane

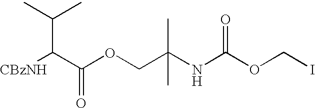

### a) 2-(N-tert.-butyloxycarbonylamino)-2-methyl-1-(N-benzyloxycarbonyl-L-valyloxy)-propane

To a cooled solution of 2-(N-(tert.-butyloxycarbonyl)-amino)-2-methylpropan-1-ol (J. Am. Chem. Soc 113 (1991) p 8883) (4.73 g, 25 mmole), 4-dimethylamino-pyridine (0.61 g, 5 mmole) and N-benzyloxycarbonyl-L-valine (6.28 g, 25 mmole) in dichloromethane (70 ml) was added dicyclohexyl-carbodiimide (6.19 g, 30 mmole) and the mixture was stirred 2 days at roommtemperature. The mixture was cooled, the urethane was filtered and the solution evaporated under reduced pressure. Ethyl acetate (200 ml) was added and the organic phase was washed twice with 5% acetic acid, 5% sodium hydrogencarbonate and water. The organic phase was dried with, sodium sulfate and evaporated under reduced pressure. The product was isolated by silica gel column chromatography with hexane/ethyl acetate. Yield: 10.2 g=96%.

### b) 2-amino-2-methyl-1-(N-benzyloxycarbonyl-L-valyloxy)-propane

To a solution of 2-(N-(tert.-butyloxycarbonyl)-amino)-2-methyl-1-(N-benzyloxycarbonyl-L-valyloxy)-propane (10 g, 23 mmole) in dichloromethane (150 ml) was added trifluoroacetic acid (30 ml) and the mixture was stirred for 1 hour at room temperature. The solution was evaporated under reduced pressure and 10% sodium carbonate solution was added. The product was extracted four times with dichloromethane, dried with sodium sulfate and evaporated under reduced pressure. The product was isolated by silica gel column chromatography with dichloromethane/methanol. Yield: 3.0 g=40% (crude)

### c) 2-(N-(chloromethoxycarbonyl)-amino)-2-methyl-1-(N-benzyloxycarbonyl-L-valyloxy)-propane

To a solution of 2-amino-2-methyl-1-(N-benzyloxycarbonyl-L-valyloxy)-propane (2.9 g, 9 mmole) and pyridine (2 ml) in dichloromethane (50 ml) was added chloromethyl chloroformate(1.55 g, 12 mmole) and the mixture was stirred for 3 hours at room temperature. The mixture was evaporated under reduced pressure and ethyl acetate was added. The organic phase was washed with water, dried with sodium sulfate and evaporated under reduced pressure. The product was isolated by silica gel column chromatography with hexane/ethyl acetate. Yield: 1.1 g=29%.

### d) 2-(N-(iodomethoxycarbonyl)-amino)-2-methyl-1-(N-benzyloxycarbonyl-L-valyloxy)-propane

To a solution of 2-(N-(chloromethoxycarbonyl)-amino)-2-methyl-1-(N-benzyloxycarbonyl-L-valyloxy)propane (1.05 g, 2.53 mmole) in dry acetone (20 ml) was added sodium iodide (1.8 g, 12 mmole) and the mixture was stirred for 36 hours at room temperature. The mixture was evaporated under reduced pressure and ethyl acetate and water were added. The organic phase was washed with 10% sodium thiosulfate solution and water. The organic phase was dried with sodium sulfate and evaporated under reduced pressure. Yield: 1.04 g=81%.

¹H-NMR (CDCl₃) 0.92 (m, 6H) 1.35 (s, 6H) 2.10 (m,1H) 3.88 (m, 1H) 4.35 (m, 2H) 5.11 (s, 2H) 5.32 (d, 1H) 5.82 (s, 1H) 5.91 (s, 2H) 7.35 (m, 5H)

### EXAMPLE A-I-25

### 1-(2-N-CBz-L-valyloxyethyl)-6-oxo-1,6-dihydro-pyridine-3-carboxylic acid iodomethyl ester

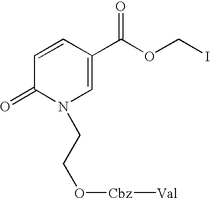

### a) 6-oxo-1,6-dihydro-pyridine-3-carboxylic acid 4-methoxybenzyl ester

To a solution of 6-hydroxynicotinic acid (4.87 g, 35 mmol) in DMF (100 mL) at room temperature, was added potassium tert-butoxide (3.93 g, 35 mmol). The reaction mixture was stirred at 60° C. for 1 h. 4-Methoxybenzylchloride (8.30 g, 53 mmol) was added and the reaction mixture was stirred at 60° C. for 4 h. The DMF was evaporated under vacuum, the residue was dissolved in ether (200 mL) and washed with water (3×100 mL). The organic phase was dried with Na₂SO₄ and evaporated to give 4.41 g of 6-oxo-1,6-dihydro-pyridine-3-carboxylic acid 4-methoxybenzyl ester.

### b) 1-(2-Hydroxyethyl)-6-oxo-1,6-dihydro-pyridine-3-carboxylic acid 4-methoxybenzyl ester

To a solution of 6-oxo-1,6-dihydro-pyridine-3-carboxylic acid 4-methoxybenzyl ester (4.41 g, 17 mmol) and K₂CO₃ (2.58 g, 18.7 mmol) in DMF (100 mL) at room temperature, was added 2-bromoethanol (2.02 g, 16.2 mmol). The reaction mixture was stirred at 80° C. for 30 h, whereupon the DMF was evaporated under vacuum. The crude product was column chromatographed (silica gel, 2→5% MeOH in CH₂Cl₂), to give 3.91 g of 1-(2-hydroxyethyl)-6-oxo-1,6-dihydro-pyridine-3-carboxylic acid 4-methoxybenzyl ester.

### c) 1-(2-N-CBz-L-valyloxyethyl)-6-oxo-1,6-dihydro-pyridine-3-carboxylic acid 4-methoxybenzyl ester

To a mixture of DCC (5.06 g, 24.5 mmol), DMAP (318 mg, 2.6 mmol) and N-CBz-L-valine (6.48 g, 25.8 mmol) in CH₂Cl₂ (200 mL) at 0° C., was added dropwise a solution of 1-(2-hydroxyethyl)-6-oxo-1,6-dihydro-pyridine-3-carboxylic acid 4-methoxybenzyl ester (6.40 g, 24 mmol) in CH₂Cl₂ (200 mL). After 1 h at 0° C., the temperature of the reaction mixture was allowed to assume room temperature and then the mixture was stirred for 5 h at room temperature. The mixture was filtered through a glass filter and the solvent was removed under reduced pressure. The crude product was column chromatographed (silica gel, 2→5% MeOH in CH₂Cl₂), to give 6.81 g 1-(2-N-CBz-L-valyloxyethyl)-6-oxo-1,6-dihydro-pyridine-3-carboxylic acid 4-methoxybenzyl ester.

### d) 1-(2-N-CBz-L-valyloxyethyl)-2-pyridone-5-carboxylic acid

To a solution of 1-(2-N-CBz-L-valyloxyethyl)-6-oxo-1,6-dihydro-pyridine-3-carboxylic acid 4-methoxybenzyl ester (6.46 g, 12 mmol) in CH₂Cl₂ (85 mL) at room temperature, was added trifluoroacetic acid (15 mL). After 1 h at room temperature, the reaction mixture was concentrated under reduced pressure. The crude product was column chromatographed (silica gel, 3→6% MeOH in CH₂Cl₂), to give 4.91 g 1-(2-N-CBz-L-valyloxyethyl)-2-pyridone-5-carboxylic acid.

¹H-NMR (CDCl₃): 12.15 (br s, 1H), 8.29 (d, J 2.2 Hz, 1H), 7.93 (dd, J=9.5, 2.2 Hz, 1H), 7.31 (m, 5H), 6.69 (d, J 9.5 Hz, 1H), 5.53 (d, 1H), 5.07 (s, 2H), 4.52-4.05 (m, 5H), 2.20-2.00 (m, 1H), 0.90 (d, 3H), 0.81 (d, 3H).

### e) 1-(2-N-CBz-L-valyloxyethyl)-6-oxo-1,6-dihydro-pyridine-3-carboxylic acid chloromethyl ester

To a solution of 1-(2-N-CBz-L-valyloxyethyl)-2-pyridone-5-carboxylic acid (4.91 g, 11.8 mmol) in dioxane (200 mL), was added dropwise a 40% aqueous solution of tetrabutylammonium hydroxide (7.65 g). After stirring for 5 min, the solution was evaporated to dryness through co-evaporation with dioxane and toluene. The residue was dissolved in dichloromethane (200 mL) and then chloroiodomethane (8.74 mL, 120 mmol) was added and the solution was stirred for 12 h at room temperature. The solution was concentrated under reduced pressure and the residue was shaken with hexane/ethyl acetate (1:1 v/v, 200 mL). The yellow crystalline solid was filtered off and the filtrate was washed with aqueous solution of sodium thiosulfate (0.1 M) and the filtered through anhydrous sodium sulfate and evaporated to dryness. The residue was column chromatographed (silica gel, 2-4% MeOH in CH₂Cl₂), to give 1.80 g of 1-(2-N-CBz-L-valyloxyethyl )-6-oxo-1,6-dihydro-pyridine-3-carboxylic acid chloromethyl ester.

### f) 1-(2-N-CBz-L-valyloxyethyl)-6-oxo-1,6-dihydro-pyridine-3-carboxylic acid iodomethyl ester

To a solution of 1-(2-N-CBz-L-valyloxyethyl)-6-oxo-1,6-dihydro-pyridine-3-carboxylic acid chloromethyl ester (1.80 g, 3.87 mmol) in acetonitrile (30 mL), was added sodium iodide (2.32 g, 15.5 mmol). The solution was stirred for4 h at 60° C. The resulting suspension was filtered and the filtrate was evaporated. The residue was dissolved in CH₂Cl₂ and washed with aqueous sodium thiosulfate (0.1 M). The organic phase was dried (Na₂SO₄) and concentrated under reduced pressure. The crude product was column chromatographed (silica gel, 1% MeOH in CH₂Cl₂), to give 2.04 g 1-(2-N-CBz-L-valyloxyethyl)-6-oxo-1,6-dihydro-pyridine-3-carboxylic acid iodomethyl ester.

¹H-NMR (CDCl₃): 8.19 (d, J=2.5 Hz, 1H), 7.79 (dd, J=9.6, 2.5 Hz, 1H), 7.32 (m, 5H), 6.52 (d, J=9.6 Hz, 1H), 6.04 (s, 2H), 5.38 (d, 1H), 5.07 (s, 2H), 4.54-4.06 (m, 5H), 2.20-2.00 (m, 1H), 0.91 (d, 3H), 0.81 (d, 3H).

### EXAMPLE A-I-26

### Iodomethyl 5-[(N-benzyloxycarbonyl-L-valyloxy)methyl]-2-furoate

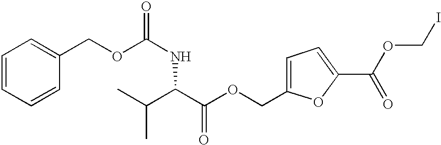

### (a) 5-[(N-Benzyloxycarbonyl-L-valyloxy)methyl]-2-furaldehyde

A solution of 5-(hydroxymethyl)-2-furaldehyde (1.00 g, 7.69 mmol) in 5 mL dry CH₂Cl₂ was added to a mixture of N-benzyloxycarbonyl-L-valine (2.40 g, 9.57 mmol), N,N′-dicyclohexylcarbodiimide (2.00 g, 9.69 mmol), and 4-dimethylaminopyridine (117 mg, 0.96 mmol) in 45 mL CH₂Cl₂. After stirring overnight, the reaction slurry was filtered, concentrated under vacuum, and subjected to flash column chromatography (silica, 2/1 petroleum ether-ethyl acetate to give the valine ester (quantitative yield).

### (b) 5-[(N-Benzyloxycarbonyl-L-valyloxy)methyl]-2-furoic acid

A solution of NaClO₂ (2.8 mmol) in 3 mL water was added dropwise to a stirred solution of 5-[(N-benzyloxycarbonyl-L-valyloxy)methyl]-2-furaldehyde (798 mg, 2.22 mmol) from step (a) in 3 mL MeCN, with cooling in an ice bath. After 2.5 h, the ice bath was removed, 2 mL more MeCN was added, and the two-phase liquid reaction mixture was stirred at room temperature for 25 h. The reaction mixture was diluted with water, made basic with saturated NaHCO₃, and extracted with ethyl acetate (3×50 mL). The separated aqueous solution was acidified to pH 2 with 5% aqueous HCl and extracted with ethyl acetate (3×50 mL). This second ethyl acetate solution was washed with brine, dried over anhydrous Na₂SO₄, and evaporated to dryness under vacuum to give the carboxylic acid (287 mg, 34%) which was used in the next step without further purification.

¹H NMR (250 MHz, CDCl₃) 0.84 and 0.93 (2d, 3H each, J=6.8 Hz), 2.15 (m, 1H), 4.35 (dd, 1H, J=9.0, 4.7 Hz), 5.10-5.24 (m, 4H), 5.44 (d, 1H, J=9.0 Hz), 6.54 (d, 1H, J=3.3 Hz), 7.23 (d, 1H, J=3.3 Hz), 7.33 (s, 5H), 11.05 (br s, 1H).

### (c) Chloromethyl 5-[(N-benzyloxycarbonyl-L-valyloxy)methyl]-2-furoate

Tetrabutylammonium hydroxide (40 wt. % solution in water, 0.55 mL, 0.84 mmol) was added to the carboxylic acid (286 mg, 0.76 mmol) from step (b) in 5 mL dioxane. The yellow solution was concentrated under vacuum, coevaporating several times with dioxane, toluene, and, lastly, CH₂Cl₂. The residue was charged with 10 mL dry CH₂Cl₂ and chloroiodomethane (0.55 mL, 7.55 mmol) was added. After stirring for 20.5 h, the reaction mixture was concentrated and subjected to flash column chromatography (silica, 2/1 petroleum ether-ethyl acetate) to give the chloromethyl ester (137 mg, 42%).

### (d) Iodomethyl 5-[(N-benzyloxycarbonyl-L-valyloxy)methyl]-2-furoate

All of the chloromethyl ester (137 mg, 0.32 mmol) from step c) was refluxed with Nal (195 mg, 1.3 mmol) in 3.2 mL dry MeCN at 70° C. for 4 h. The solvent was removed under vacuum and the residue was subjected to flash column chromatography (silica, 311 petroleum ether-ethyl acetate) to give the iodomethyl ester (1 52 mg, 92%).

¹H NMR (250 MHz, CDCl₃) 0.84and 0.93 (2d, 3H each, J=6.8 Hz), 2.16 (m, 1H), 4.33 (dd, 1H, J=9.1, 4.7 Hz), 5.09-5.21 (m, 4H), 5.36 (d, 1H, J=9.1 Hz), 6.08 (s, 2H), 6.52 (d, 1H, J=3.4 Hz), 7.19 (d, 1H, J=3.5 Hz), 7.33 (s, 5H).

### EXAMPLE A-I-27

### 4-(2-N-benzyloxycarbonyl-L-valyloxyethyl) benzoic acid

### a) 4-Methoxybenzyl 4-(2-hydroxyethoxy)benzoate

To a solution of 4-methoxybenzyl 4-hydroxybenzoate (7.0 g, 27 mmole) in dry N,N-dimethylformamide (50 ml) was added potassium carbonate (4.15 g, 30 mmole) and 2-bromoethanol. The mixture was stirred 48 hours at 80° C., evaporated under reduced pressure and ethyl acetate and water were added. The organic phase was washed five times with water and dried with sodium sulfate. The solution was evaporated under reduced pressure and the product was isolated by silica gel column chromatography with hexane/ethyl acetate. Yield: 6.8 g=83%.

### b) 4-methoxybenzyl 4-(2-N-benzyloxycarbonyl-L-valyloxyethoxy)benzoate

To a solution of 4-methoxybenzyl 4-(2-hydroxyethoxy)benzoate (6.6 g, 21.8 mmole), 4-dimethylaminopyridine (0.61 g, 5 mmole) and N-benzyloxycarbonyl-L-valine (6.3 g, 25 mmole) in dichloromethane (80 ml) was added dicyclohexyl-carbodiimide (5.2 g, 25 mmole) and the mixture was stirred overnight at room temperature. The mixture was cooled and the urethane was filtered. The solution was evaporated and ethyl acetate (200 ml) was added. The organic phase was washed twice with 5% acetic acid, 5% sodium hydrogencarbonate and water. The organic phase was dried with sodium sulfate and evaporated under reduced pressure. The product was isolated by silica gel column chromatography with dichloromethane/methanol. Yield: 10.6 g=90%.

### c) 4-(2-N-benzyloxycarbonyl-L-valyloxyethoxy)-benzoic acid

To a solution of 4-methoxybenzyl 4-(2-N-benzyloxycarbonyl-L-valyloxyethoxy) benzoate (10.2 g, 19.04 mmole) in dichloromethane (100 ml) was added trifluoroacetic acid (20 ml) and the mixture was stirred 3 hours at room temperature. The solution was evaporated under reduced pressure and co-evaporated two times with toluene. The product was isolated by silica gel column chromatography. Yield: 6.9 g=87%. The product may be activated and esterified direct to a drug or converted to iodomethyl 4-(2-N-benzyloxycarbonyl-L-valyloxyethoxy)-benzoic acid as described above, that is by treatment with a base, chloroiodomethane, separation and then treatment with Nal.

¹H-NMR (CDCl₃) 0.94 (m, 6H) 2.18 (m, 1H) 4.22-4.68 (m, 5H) 5.10 (s, 2H) 6.94 (d, 2H) 7.35 (m, 5H) 8.05 (d, 2H)

Preparation of Compounds of the Invention

### EXAMPLE 1

### (1S, 2S)-N-{cis-2-[6-fluoro-2-(L-isoleucyloxymethyloxy)-3-propionylphenyl]cyclopropyl}-N′-[2-(5-cyanopyridyl)]urea

### a) (1S, 2S)-N-{cis-2-[6-fluoro-2-(N-BOC-L-isoleucyloxymethyloxy)-3-propionylphenyl]cyclopropyl}-N′-[2-(5-cyanopyridyl)]urea.

To a solution of (1S, 2S)-N-{cis-2-[6-fluoro-2-hydroxy-3-propionylphenyl]cyclopropyl}-N′-[2-(5-cyanopyridyl)]urea (2.03 g, 5.5 mmol) in THF (50 mL) at 20° C., was added NaH (60%, 220 mg, 5.5 mmol). After the mixture was stirred 1.5 h at 20° C., N-BOC-L-isoleucine iodomethyl ester (16.5 g, 16.5 mmol) was added. The solution was stirred for 6 h at room temperature and then concentrated under reduced pressure. The crude product was column chromatographed (aluminium oxide 90, 1% MeOH in CH₂Cl₂), to give 1.76 g of the title product.

¹H-NMR (CDCl₃): 9.75 (br s, 1H), 9.15 (br s, 1H), 8.16 (s, 1H), 7.71 (dd, 1H), 7.52 (dd, 1H), 7.00-6.87 (m, 2H), 5.81 (d, 1H), 5.68 (d, 1H), 5.00 (d, 1H), 4.21 (dd, 1H), 3.40-3.25 (m, 1H), 2.99-2.72 (m, 2H), 2.10 (dd, 1H), 1.85-1.68 (m, 1H), 1.60-1.47 (m, 1H), 1.41 (s, 9H), 1.32-1.05 (m, 3H), 1.13 (t, 3H), 0.88-0.78 (m, 6H).

### b) (1S, 2S)-N-{cis-2-[6-fluoro-2-(L-isoleucyloxymethyloxy)-3-propionylphenyl]cyclopropyl}-N′-[2-(5-cyanopyridyl)]urea

To TFA (30 mL) at 0° C., was added (1S, 2S)-N-{cis-2-[6-fluoro-2-(N-BOC-L-isoleucyloxymethyloxy)-3-propionylphenyl]cyclopropyl}-N′-[2-(5-cyanopyridyl)]urea(1.81 g, 2.96 mmol). The reaction mixture was stirred at 0° C. for 30 min and then concentrated under reduced pressure at 0° C. The crude product was column chromatographed (silica gel, 10% MeOH in CH₂Cl₂), to give 1.48 g of the title compound as the TFA-salt.

¹H-NMR (CDCl₃): 9.50 (br s, 1H), 9.42 (br s, 1H), 8.34 (s, 1H), 7.73 (dd, 1H), 7.27 (m, 1H), 7.10 (d, 1H), 6.81 (dd, 1H), 6.16 (d, 1H), 5.73 (d, 1H), 3.87 (d, 1H), 3.39 (m, 1H), 3.05-2.68 (m, 2H), 2.29 (dd, 1H), 2.10-1.88 (m, 2H), 1.57-1.21 (m, 3H), 1.09 (t, 3H), 1.02 (d, 3H), 0.91 (t, 3H).

### EXAMPLE 2

### (1S, 2S)-N-{cis-2[6-fluoro-2-(L-valyloxymethyloxy)-3-propionylphenyl)]cyclopropyl}-N′-[2-(5-cyanopyridyl)]urea

### a) (1S, 2S)-N-{cis-2-[6-fluoro-2-(N-CBz-L-valyloxymethyloxy)-3-propionylphenyl)]cyclopropyl}-N′-[2-(5-cyanopyridyl)]urea

To a solution of (1S, 2S)-N-{cis-2-[6fluoro-2-hydroxy-3-propionylphenyl]cyclopropyl}-N′-[2-(5-cyanopyridyl)]urea (368 mg, 1 mmole) in THF (5 ml) was added sodium hydride in paraffin (60%, 38 mg, 0.95 mmole). After 1.5 hour, N-CBz-L-valyloxymethyl iodide (1.09 g, 2.8 mmole) prepared analogously to the N-BOC-L-isoleucyloxymethyl iodide described above was added to the solution and reaction was kept 18 hours. The mixture was filtered through Celite and poured into sodium hydrogen carbonate aqueous solution, and extracted with methylene chloride. The organic phase was dried and the product was isolated with silica gel column chromatography to yield 210 mg.

¹H-NMR (CDCl₃): 8.16 (s, 1H), 7.70 (dd, 1H), 7.49 (t, 1H), 7.35 (m, 5H), 6.93 (m, 2H), 5.78 (dd, 2H), 5.27 (d, 1H), 5.11 (s, 2H), 4.28 (m, 1H), 3.34 (m, 1H), 2.84 (m, 2H), 2.09 (m, 2H), 1.54 (m, 1H), 1.34 (m, 1H), 1.10 (t, 3H), 0.87 (dd, 6H).

### b) (1S, 2S)-N-{cis-2-[-fluoro-2-(L-valyloxymethyloxy)-3-propionylphenyl)]cyclopropyl}-N′-[2-(5-cyanopyridyl)]urea

(1S, 2S)-N-{cis-2-[6-fluoro-2-(N-CBz-L-valyloxymethyloxy)-3-propionylphenyl)]cyclopropyl}-N′-[2-(5-cyanopyridyl)]urea (200 mg, 0.32 mmole) was dissolved in a mixed solvent of methanol (5 ml), ethylacetate (2 ml) and acetic acid (1 ml). To the solution was added palladium black (35 mg). It was kept under hydrogen at atmospheric pressure for two hours. After filtration, the solution was evaporated and the product was purified by silica gel column chromatography yielding 66 mg.

¹H-NMR (CDCl₃) 8.20 (d, 1H), 7.73 (dd, 1H), 7.44 (dd, 1H), 6.94 (m, 2H), 5.80 (dd, 2H), 3.37 (1H), 2.88 (m, 2H), 2.10 (m, 2H), 1.60 (m, 1H), 1.46 (m, 1H), 1.08 (t, 3H), 0.94 (m, 6H).

### EXAMPLE 3

### (1S, 2S)-N-{cis-2-[6-fluoro-2-(2,2-dimethyl-3-(L-valyloxy)-propionyloxymethyloxy) -3-propionylphenyl-cyclopropyl}-N′-[2-(5-cyanopyridyl)]urea

### a) (1S, 2S)-N-{cis-2-[6-fluoro-2-(2,2-dimethyl-3-(N-Boc-L-valyloxy)propionyloxymethyloxy) -3-propionylphenyl]cyclopropyl}-N′-[2-(5-cyanopyridyl)]urea.

To a solution of (1S, 2S)-N-[cis-2-(6-fluoro-2-hydroxy-3-propionylphenyl)cyclopropyl]-N′-[2-(5-cyanopyridyl)]urea (368 mg, 1 mmole) in THF (5 ml) was added sodium hydride in paraffin (60%, 38 mg, 0.95 mmole). After one hour, 2,2-dimethyl-3-(N-Boc-L-valyloxy)propionic acid iodomethyl ester (1.35 g, 3 mmole) was added to the solution. After 5 hr at room temperature, it was then raised to 50° C. and reaction was kept 18 hours. The reaction mixture was poured into sodium hydrogen carbonate aqueous solution and extracted with methylene chloride. The organic phase was dried and the product was isolated with alumina column chromatography. 140 mg.

¹H-NMR (CDCl₃): 8.39-6.70 (m, 5H) 5.77 (m, 2H ) 5.15 (d, 1H) 4.00 (m, 3H) 3.40 (m, 1H) 2.90 (m, 2H) 2.30 (m, 1H) 2.20 (m, 1H) 1.70 (m, 1H) 1.42 (s, 9H) 1.16 (d, 6H) 0.92 (m, 9H)

### b) (1S, 2S)-N-{cis-2-[6-fluoro-2-(2,2-dimethyl-3-(L-valyloxy)propionyl-oxymethyloxy)-3-propionylphenyl]-cyclopropyl}-N′-[2-(5-cyanopyridyl)]urea

(1S, 2S )-N-{cis-2-[6-fluoro-2-(2,2-dimethyl-3-(N-Boc-L-valyloxy)-propionyloxymethyloxy)-3-propionylphenyl)]cyclopropyl}-N′-[2-(5-cyanopyridyl)]urea (120 mg) was treated with trifluoroacetic acid at 0° C. for 20 min. The solution was evaporated and coevaporated with toluene and methanol succesively, giving the titled product in quantitative yield.

¹H-NMR (CDCl₃): 8.33 (d, 1H) 7.89 (d, 1H) 7.48 (t, 1H) 7.16 (m, 1H) 6.96 (t, 1H) 5.70 (dd, 2H) 4.18 (dd, 2H) 4.01 (m, 1H) 3.38 (m, 1H) 2.88 (m, 2H) 2.16 (m, 1H) 1.58 (m, 2H) 1.25 (d, 6H) 1.04 (m, 9H).

### EXAMPLE 4

### (1S,2S)-N-{cis-2-[6-fluoro-2-(3,3-bis-(L-valyloxymethyl)propionyloxymethyloxy)-3-propionylphenyl]cyclopropyl}-N′-[2-(5-cyanopyridyl)]urea

### a) (1S, 2S)-N-{cis-2-[6-fluoro-2-(3,3-bis(N-CBz-L-valyloxymethyl)propionyloxymethyloxy)-3-propionylphenyl)]cyclopropyl}-N′-[2-(5-cyanopyridyl)]urea

To a solution of (1S, 2S)-N-{cis-2-[6-fluoro-2-hydroxy-3-propionylphenyl]cyclopropyl}-N′-[2-(5-cyanopyridyl)]urea (331 mg, 1 mmole) in THF (5 ml) was added sodium hydride in paraffin (60%, 32 mg, 0.81 mmole). After one hour, 3,3-bis-(N-CBz-L-valyloxymethyl) propionic acid iodomethyl ester (1.3 g, 1.8 mmole) was added to the solution. After 5 hr at room temperature, it was then raised to 50° C. and reaction was kept 18 hours. The mixture was poured into sodium hydrogen carbonate aqueous solution, and extracted with methylene chloride. The organic phase was dried and the product was isolated with alumina column chromatography. 185 mg.

¹H-NMR (CDCl₃): 8.19 (s, 1H) 7.89 (dd, 1H) 7.32 (m, 11H) 7.10 (m, 1H) 6.90 (t, 1H) 5.79 (dd, 2H) 5.09 (s, 2H) 4.31 (m, 2H) 4.08 (m, 4H) 2.95 (m, 2H) 2.50 (m, 3H) 2.17 (m, 3H) 1.55 (m, 1H) 1.07 (t, 3H) 0.88 (dd, 12H).

### b) (1S, 2S)-N-{cis-2-[6-fluoro-2-(3,3-bis(L-valyloxymethyl)propionyloxymethyloxy)-3-propionylphenyl]cyclopropyl}-N′-[2-(5-cyanopyridyl)]urea

(1S, 2S)-N-{cis-2-[6-fluoro-2-(3,3-bis(N-CBz-L-valyloxymethyl)propionyloxymethyloxy)-3-propionylphenyl]cyclopropyl}-N′-[2-(5-cyanopyridyl)]urea (170 mg, 0.17 mmole) was dissolved in a mixed solvent of methanol (5 ml), ethyl acetate (2 ml) and acetic acid (1 ml). To the solution was added palladium black (30 mg). It was kept under hydrogen at atmospheric pressure for four hours. After filtration, the solution was evaporated and the product was purified by silica gel column chromatography. 80 mg.

¹H-NMR (DMSO-d6): 8.38 (d, 1H) 8.02 (d, 1H) 7.42 (m, 2H) 7.12 (t, 1H ) 5.70 (dd, 2H) 4.00 (s, 4H) 3.16 (m, 1H) 3.08 (d, 2H) 2.80 (m, 1H) 2.40 (m, 2H), 2.11 (m, 1H) 1.52 (m, 1H) 0.95 (t, 3H) 0.98 (dd, 12H).

### EXAMPLE 5

### (1S,2S)-N-{cis-2-[6-fluoro-2-(2-(L-valyloxy)-ethoxycarbonyloxymethyloxy)-3-propionylphenyl)]cyclopropyl}-N′-[2-(5-cyanopyridyl)]urea

### a) (1S, 2S)-N-{cis-2-[6-fluoro-2-(2-(N-CBz-L-valyloxy)-ethoxycarbonyloxymethyloxy)-3-propionylphenyl)]cyclopropyl}-N′-[2-(5-cyanopyridyl)]urea.

To a solution of (1S, 2S)-N-{cis-2[6-fluoro-2-hydroxy-3-propionylphenyl]cyclopropyl}-N′-[2-(5-cyanopyridyl)]urea (368 mg, 1 mmole) in THF (5 ml) was added sodium hydride in paraffin (60%, 38 mg, 0.95 mmole). After 1.5 hr, 2-(N-CBz-L-valyloxy)ethoxycarbonyloxymethyl iodide (864 mg, 1.7 mmole) was added to the solution. The reaction was kept for 48 hours. The mixture was poured into sodium hydrogen carbonate aqueous solution, and extracted with methylene chloride. The organic phase was dried and the product was isolated with silica gel column chromatography. 210 mg.

¹H-NMR (CDCl₃): 8.21 (d, 1H) 7.72 (d, 1H) 7.28 (m, 6H) 6.90 (m, 2H) 5.75 (dd, 2H) 5.09 (s, 2H) 4.35 (m, 4H) 2.85 (m, 2H) 2.50 (m, 2H) 2.16 (m, 1H), 1.65 (m, 1H) 1.11 (t, 3H) 0.93 (dd, 6H).

### b) 1S, 2S)-N-{cis-2-[6-fluoro-2-(2-(L-valyloxy)-ethoxycarbonyloxymethyloxy)-3-propionylphenyl)]cyclopropyl}-N′-[2-(5-cyanopyridyl)]urea.

(1S, 2S)-N-{cis-2-[6-fluoro-2-(2-(N-CBz-L-valyloxy)-ethoxycarbonyloxymethyloxy)-3-propionylphenyl)]cyclopropyl}-N′-[2-(5-cyanopyridyl)]urea is deprotected by conventional techniques such as palladium black in a mixed solvent of methanol, ethyl acetate and acetic acid under hydrogen at atmospheric pressure followed by conventional work up such as filtration, evaporation and silica gel column chromatography.

### EXAMPLE 6

### (1S,2S)-N-[cis-2-(6-fluoro-2-(1,3-bis-L-valyloxy-2-(propoxycarbonyloxy-methyloxy)-3-propionylphenyl)cyclopropyl]-N′-[2-(5-cyanopyridyl)]urea

### a) (1S,2S)-N-[cis-2-(6-fluoro-2-(1,3-bis-(N-BOC-L-valyloxy-2-(propoxycarbonyloxymethyloxy)-3-propionylphenyl)cyclopropyl]-N′-[2-(5-cyanopyridyl)]urea

NaH (121 mg, 60% w/w in mineral oil, 3.0 mmol) was added to a mixture of (1S,2S)-N-[cis-2-(6-fluoro-2-hydroxy-3-propionylphenyl)cyclopropyl]-N′-[2-(5-cyanopyridyl)]urea (1.05 g, 2.85 mmol) in 15 mL dry THF under N₂. After 1 h, the solution was concentrated to dryness and redissolved in 10 mL DMF. 2-O-iodomethoxycarbonyl-1,3-di-O-(N-tert-butoxycarbonyl-L-valyl)glycerol (2.96 g, 4.39 mmol) in 15 mL DMF was added and the reaction mixture was stirred for 20 h. Removal of solvent under vacuum followed by flash column chromatography (silica gel, 2/1 ethyl acetate-petroleum ether) gave 1.46 g (56%) of the title product as a white solid.

¹H NMR (250 MHz, CD₃OD) 0.94 and 0.97 (2d overlap, 12H), 1.1 (t, 3H), 1.23 (m,1H), 1.46 (s, 18H), 1.64 (m, 1H), 2.07-2.24 (m, 3H), 2.90 (m, 2H), 3.32 (m, 1H), 4.06 (d, 2H), 4.28-4.52 (m, 4H), 5.13 (m, 1H), 5.78 and 5.88 (AB q, 2H), 7.07-7.19 (m, 2H), 7.62 (dd, 1H), 7.92 (dd, 1H), 8.31 (d, 1H).

### b) (1S,2S)-N-[cis-2-(6-fluoro-2-(1,3-bis-L-valyloxy-2-(propoxycarbonyloxymethyloxy)-3-propionylphenyl)cyclopropyl]-N′-[2-(5-cyanopyridyl)]urea.

Ice-cold trifluoroacetic acid (30 mL) was added to the intermediate of step a (1.69 g, 1.85 mmol) in an ice bath, under N₂. After 7 min, the reaction mixture was concentrated under vacuum, coevaporating several times with, initially, toluene and, finally, CH₂Cl₂. The oily residue was chromatographed immediately on a silica gel column with 10-20% methanol in CH₂Cl₂ to give 1.37 g of the product as a trifluoroacetate salt.

¹H NMR (250 MHz, CD₃OD) 1.07-1.12 (m, 15H), 1.26 (m, 1H), 1.63 (m, 1H), 2.19 (m, 1H), 2.35 (m, 2H), 2.89 (m, 2H), 4.08 (m, 2H), 4.44-4.71 (m, 4H), 5.26 (m, 1H), 5.79 and 5.91 (AB q, 2H), 7.10-7.18 (m, 2H), 7.59 (dd, 1H), 7.93 (dd, 1H), 8.30 (d, 1H).

¹⁹F NMR (235 MHz, CD₃OD) −103.5, −73.5.

### EXAMPLE 7

### (1S,2S)-N-[cis-2-(6-fluoro-2-(L-valyloxy)methoxycarbonyloxy-3-propionylphenyl)cyclopropyl]-N′-[2-(5-cyanopyridyl)]urea

### a) (1S,2S)-N-[cis-2-(6-fluoro-2-chloromethoxycarbonyloxy-3-propionylphenyl)cyclopropyl]-N′-[2-(5-cyanopyridyl)]urea

Chloromethyl chloroformate (2.3 mL, 25 mmol) was added by syringe to a mixture of (1S,2S)-N-[cis-2-(6-fluoro-2-hydroxy-3-propionylphenyl)cyclopropyl]-N′-[2-(5-cyanopyridyl)]urea (4.695 g, 12.7 mmol) and pyridine (6.1 mL, 76 mmol) in 65 mL dry CH₂Cl₂ with cooling in an ice bath, under N₂. After 10 min, the ice bath was removed and the mixture was stirred at room temperature for 1 h 40 min. The mixture was diluted with 100 mL CH₂Cl₂ and washed with 50 mL H₂O. The aqueous phase was reextracted with 25 mL CH₂Cl₂. The combined organic phases were washed with 50 mL saturated NaHCO₃, followed by 2×50 mL brine. Drying over Na₂SO₄ and concentration under vacuum gave a crude material that was subjected to flash column chromatography (silica gel, 1/1 ethyl acetate-petroleum ether) to give 4.05 g (69%) title product.

¹NMR (250 MHz, CDCl₃) 1.15 (t, 3H), 1.30 (m, 1H), 1.59 (m, 1H), 2.02 (m, 1H), 2.87 (q, 2H), 3.29 (m, 1H), 5.87 (s, 2H), 6.97 (d, 1H), 7.09 (m, 1H), 7.72 (dd, 1H), 7.76 (dd, 1H), 8.10 (dd, 1H), 9.26 (br s, 1H), 10.09 (brs, 1H).

### b) (1S,2S)-N-[cis-2-(6-fluoro-2-iodomethoxycarbonyloxy-3-propionylphenyl)cyclopropyl]-N′-[2-(5-cyanopyridyl)]urea

(1S,2S)-N-[cis-2-(6-fluoro-2-chloromethoxycarbonyloxy-3-propionylphenyl)cyclopropyl]-N′-[2-(5-cyanopyridyl)]urea (3.97 g, 8.6 mmol) and Nal (5.17 g, 34.5 mmol) in 85 mL dry acetonitrile were refluxed at 70° C. for 4 h under N₂. The solvent was removed in vacuo, the residue was partitioned between 100 mL CH₂Cl₂ and 25 mL H₂O, the aqueous phase was reextracted with 10 mL CH₂Cl₂, and the organic phases were combined, washed successively with 2×25 mL 5% Na₂S₂O₃ and 2×25 mL brine, and dried over Na₂SO₄. Flash column chromatography (silica gel, 2/1 ethyl acetate-petroleum ether) of the crude product obtained after concentration in vacuo gave 4.15 g material containing 92% of the title compound and traces of the starting material.

¹H NMR (250 MHz, CDCl₃) 1.18 (t, 3H), 1.34 (m, 1H), 1.62 (m, 1H), 2.03 (m, 1H), 2.86 (q, 2H), 3.32 (m, 1H), 6.08 (s, 2H), 6.97 (d, 1H), 7.08 (m, 1H), 7.70-7.76 (m, 2H), 8.13 (d, 1H), 8.90 (br s, 1H), 9.30 (br s, 1H).

### c) (1S,2S)-N-[cis-2-(6-fluoro-2-(N-BOC-L-valyloxy)methoxycarbonyloxy-3-propionylphenyl)cyclopropyl]-N′-[2-(5-cyanopyridyl)]urea

Tetrabutylammonium hydroxide (40 wt % solution in water, 6.4 mL, 9.8 mmol) was added to Boc-L-valine (2.54 g, 11.7 mmol) in 30 mL dioxane. The solution was concentrated in vacuo, coevaporating several times with dioxane, toluene, and CH₂Cl₂, and dried under vacuum overnight. The resulting Q salt was dissolved in 30 mL dry CH₂Cl₂ and (1S,2S)-N-[cis-2-(6-fluoro-2-(iodomethoxycarbonyloxy)-3-propionylphenyl)cyclopropyl]-N′-[2-(5-cyanopyridyl)]urea (7.1 mmol) in 65 mL dry CH₂Cl₂ was added. After stirring under N₂ for 18 h, the reaction mixture was washed with 3×50 mL H₂O, 1×50 mL 5% Na₂S₂O₃, and 2×50 mL H₂O. The organic phase was dried over Na₂SO₄, concentrated, and submitted to flash column chromatography (silica gel, 3/1 ethyl acetate-petroleum ether) to give 2.21 g (49%) product.

¹H NMR (250 MHz, CD₃OD) 0.98 (d, 3H), 1.02 (d, 3H), 1.17 (t, 3H), 1.24 (m, 1H), 1.47 (s, 9H), 1.59 (m, 1H), 2.06 (m, 1H), 2.24 (m, 1H), 2.96 (q, 2H), 3.24 (m. 1H), 4.15 (d, 1H), 5.94 and 6.02 (AB q, 2H), 7.12 (d, 1H), 7.26 (m, 1H), 7.91 (dd, 1H), 7.94 (dd, 1H), 8.23 (dd, 1H).

### d) (1S,2S)-N-[cis-2-(6-fluoro2-(L-valyloxy)methoxycarbonyloxy-3-propionylphenyl)cyclopropyl]-N′-[2-(5-cyanopyridyl)]urea

Cold trifluoroacetic acid (40 mL) was added to (1S,2S)-N-[cis-2-(6-fluoro-2-(N-BOC-L-valyloxymethoxycarbonyloxy)-3-propionylphenyl )cyclopropyl]-N′-[2-(5-cyanopyridyl)]urea (1.94 g, 3.02 mmol) with cooling in an ice bath, under N₂. After 5 min, the solution was concentrated in vacuo, coevaporating several times with toluene, and then CH₂Cl₂, and dried under vacuum for several hours to give the compound as a trifluoroacetate salt in quantitative yield.

¹H NMR (250 MHz, CD₃OD) δ1.12-1.18 (m, 9H), 1.25 (m, 1H), 1.59 (m, 1H), 2.07 (m, 1H), 2.47 (m, 1H), 2.97 (q, 2H), 3.26 (m, 1H), 4.16 (d, 1H), 6.01 and 6.37 (AB q, 2H), 7.11 (d, 1H), 7.29 (m, 1H), 7.92 (dd, 1H), 7.99 (dd, 1H), 8.22 (d, 1H).

¹⁹F NMR (235 MHz, CD₃OD) δ−102.7, −74.0.

### EXAMPLE 8

### (1S, 2S )-N-{cis-2[6-fluoro-2-(3-carboxylpropionyloxymethyloxy)-3propionylphenyl]cyclopropyl}-N′-[2-(5-cyanopyridyl)]urea

### a) (1S, 2S)-N-{cis-2-[6-fluoro-2-(3-benzyloxycarbonylpropionyloxymethyloxy)-3-propionylphenyl]cyclopropyl}-N′-[2-(5-cyanopyridyl)]urea

3-Benzyloxycarbonylpropionic acid iodomethyl ester (522 mg, 1.5 mmole) was added to a solution of (1S, 2S)-N-{cis-2-[6-fluoro-2-hydroxy-3-propionylphenyl]cyclopropyl}-N′-[2-(5-cyanopyridyl)]urea (185 mg, 0.5 mmole) in THF (5 ml) which had been treated with sodium hydride in paraffin (60%, 20 mg, 0.5 mmole) for 30 min. After 18 hr at room temperature, the reaction mixture was poured into sodium hydrogen carbonate aqueous solution, and extracted with methylene chloride. The organic phase was dried and the product was isolated with alumina column chromatography. 115 mg.

¹H-NMR (CDCl₃): 8.20 (d, 1H) 7.72 (dd, 1H) 7.49 (dd, 1H) 7.35 (m, 5H) 6.97 (m, 2H) 5.73 (dd, 2H) 5.17 (s, 2H) 3,35 (m, 1H) 2.88 (m, 2H) 2.60 (m, 4H) 2.12 (m, 1H) 1.58 (m, 1H) 1.11 (t, 3H).

### b) (1S, 2S)-N-{cis-2-[6-fluoro-2-(3-carboxylpropionyloxymethyloxy)-3-propionylphenyl]cyclopropyl}-N′-2-(5-cyanopyridyl)]urea

(1S,2S )-N-{cis-2-[6-fluoro-2-(3-carboxylpropionyloxymethyloxy)-3-propionylphenyl)]cyclopropyl}-N′-[2-(5cyanopyridyl)]urea (100 mg, 0.17 mmole) was dissolved in a mixed solvent of ethylacetate (3 ml) and acetic acid (1 ml). To the solution was added palladium black (30 mg). It was kept under hydrogen at atmospheric pressure for three hours. After filtration, the solution was evaporated and the product was purified by silica gel column chromatography. 81 mg. An Rx bearing intermediate linking group such as 1,3-bis-N-Boc-valyloxyglycerol (preparation described in WO9909031) can be esterified to the free carboxyl group using conventional esterification comditions as described in WO9909031.

¹H-NMR (CDCl₃): 8.21 (s, 1H) 7.75 (d, 1H) 7.49 (dd, 1H) 7.08 (d, 5H) 6.97 (t, 1H) 5.73 (dd, 2H) 5.17 (s, 2H) 3,26 (m, 1H) 2.87 (m, 2H) 2.60 (m, 4H) 2.09 (m, 1H) 1.58 (m, 1H) 1.11 (t, 3H)

### EXAMPLE 9

### (1S, 2S)-N-[cis-2-(6-fluoro-2-O-(4-L-valyloxybenzoyl)-3propionylphenyl)-cyclopropyl]-N′-(5-cyanopyrid-2-yl) urea

### a) 4-benzyloxybenzoic acid

To a solution of 4-hydroxybenzoic acid (6.9 g, 50 mmole) in 150 ml DMF was added potassium tert.-butoxide (12.34 g, 110 mmole) and the mixture was stirred at room temperature for one hour. Benzyl bromide (20.5 g, 120 mmole) was added and the mixture was stirred for two days at room temperature. The mixture was evaporated under reduced pressure and 100 ml 1,4-dioxane and a solution of sodium hydroxide (6.0 g, 150 mmole)in 50 ml water was added. The mixture was refluxed for two hours, cooled and evaporated under reduced pressure. Water was added and the mixture was acidified with acetic acid. The product was filtered, washed with cold water and dried. Yield: 10.2 g=89%.

### b) 4-benzyloxybenzoyl chloride

To a mixture of 4-benzyloxybenzoic acid (2.28 g, 10 mmole) in 20 ml dried dichloromethane were added five drops of DMF and 2.5 ml thionyl chloride. The mixture was refluxed for three hours and evaporated under reduced pressure. Yield: 2.45 g=100%

### c) (1S, 2H)-N-[cis-2-(6-fluoro-2-O-(4-benzyloxybenzoyl)-3-propionylphenyl)cyclopropyl]-N′-[2-(5-cyanopyrid-2-yl) urea

To a solution of (1S, 2S)-N-[cis-2-(6-fluoro-2-hydroxy-3-propionylphenyl) cyclopropyl]-N′-(5-cyanopyrid-2-yl)urea (184 mg, 0.5 mmole) in 3 ml DMF was added potassium tert. butoxide (78.5 mg, 0.7 mmole) and the mixture was stirred for one hour at room temperature. A solution of 4-benzyloxybenzoylchloride (185 mg, 0.75 mmole) in 1 ml DMF was added and the mixture was stirred overnight at room temperature. 40 ml ethyl acetate were added and the organic phase was washed four times with water. The solution was dried with sodium sulfate and evaporated under reduced pressure. The product was isolated by silica gel column chromatography. Yield: 180 mg=62%.

¹H-NMR (DMSO δ-6) 0.92 (m, 4H) 1.31(m, 1H) 1.85 (m, 1H) 2.82 (m, 2H) 3.06 (m, 1H) 5.26 (s, 2H) 7.20 (m 2H) 7.38-8.12 (m, 11H) 8.38 (m, 1H)

### d) (1S, 2S)-N-[cis-2-(6-fluoro-2-O(4-hydroxybenzoyl)-3-propionylphenyl)cyclopropyl]-N′-(5-cyanopyrid-2-yl)]urea-O-4-hydroxybenzoate

A solution of the intermediate of step c) (170 mg, 0.29 mmole) in 15 ml ethyl acetate and 15 ml methanol was hydrogenated with 10% palladium on charcoal (30 mg) three times at room temperature and normal pressure. The catalyst was filtered and washed with ethyl acetate and methanol and the solution was evaporated under reduced pressure. The product was isolated by silica gel column chromatography. Yield: 100 mg=70%.

¹H-NMR (DMSO δ-6) 0.93 (m, 4H) 1.32 (m, 1H) 1.88 (m, 1H) 2.85 (m, 2H) 3.05 (m, 1H) 6.92 (m, 2H) 7.38 (m, 2H) 8.00 (m, 4H) 8.38 (m, 1H)

### e) (1S, 2S)-N-[cis-2-(6-fluoro-2-O(4-L-valyloxybenzoyl)-3-propionylphenyl)-cyclopropyl]-N′-(5-cyanopyrid-2-yl) urea

An R₂ group, such as N-protected L-valyl is acylated to the exposed ring hydroxy group using conventional acylation conditions as described herein and deprotected to yield a compound of the invention.

### EXAMPLE 10

### (1S, 2S)-N-[cis-2-(6-fluoro-2-O((4-isoleucyloxybenzoyloxymethy)-3-propionylphenyl)-cyclopropyl]-N′-[2-(5-cyanopyridyl)]urea-O-methylene-4-hydroxybenzoate-O-L-isoleucyl ester

### a) Methyl-4-(4-methoxybenzyloxy)benzoate

To a solution of methyl 4-hydoxybenzoate (6.85 g, 45 mmole) in 80 ml DMF was added potassium tert.butoxide (5.6 g, 51 mmole) and the mixture was stirred at room temperature for one hour. 4-Methoxybenzyl chloride (8.3 g, 52 mmole) was added and the mixture was stirred overnight at room temperature. The mixture was evaporated under reduced pressure and 200 ml ethyl acetate was added. The organic phase was washed four times with water, dried with sodium sulfate and evaporated under reduced pressure. Yield: 12.3 g=100%

¹H-NMR (CDCl₃) 3.82 (s, 3H) 3.88 (s, 3H) 5.03 (s, 2H) 6.96 (m, 4H) 7.36 (d, 2H) 7.98 (d, 2H)

### b) 4-(4-methoxybenzyloxy) benzoic acid

To a solution of methyl 4-(4-methoxybenzyloxy)benzoate (12.2 g, 44.8 mmole) in 50 ml 1,4-dioxane was added a solution of lithium hydroxide (2.15 g, 89,6 mmole) and the mixture was stirred overnight at 60° C. The mixture was evaporated under reduced pressure and 5% acetic acid was added. The product was filtered, washed with water and dried. Yield: 10.1 g=87%

¹H-NMR (DMSO δ-6) 3.74 (s, 3H) 5.08 (s, 2H) 6.92 (d, 2H) 7.06 (d, 2H) 7.36 (d, 2H) 7.90 (d, 2H)

### c) Chloromethyl 4-(4-methoxybenzyloxy)benzoate

To a solution of 4-(4-methoxybenzyloxy) benzoic acid (5.16 g, 20 mmole) in 100 ml 1,4-dioxane was added a 40% solution of tetrabutylammonium hydroxide (14.27 g, 22 mmole) and the mixture was stirred 2 hours at room temperature. The mixture was evaporated under reduced pressure and co-evaporated two times with 1,4-dioxane and two times with toluene. The dried product was dissolved in 60 ml dichloromethane and iodochloromethane (35.3 g 200 mmole) was added. The solution was stirred for two days at room temperature and evaporated under reduced pressure. About 100 ml ethyl actate was added and the organic phase washed twice with water, dried with sodium sulfate and evaporated under reduced pressure. The product was isolated by silica gel column chromatography. Yield: 4.48 g=73%

¹H-NMR (CDCl₃) 3.83 (s, 3H) 5.06 (s, 2H) 5.94 (s, 2H) 7.00 (m, 4H) 7.36 (d, 2H) 8.05 (d, 2H)

### d) Iodomethyl 4-(4-methoxybenzyloxy)benzoate

To a solution of chloromethyl 4-(4-methoxybenzyloxy) benzoate (0.77 g, 2.5 mmole) in 15 ml dry acetone was added sodium iodide (1.87 g, 12.5 mmole) and the mixture was stirred overnight at room temperature. The mixture was evaporated under reduced pressure and extracted with ethyl actate/water. The organic phase was washed with a 5% sodium thiosulfate solution, dried with sodium sulfate and evaporated under reduced pressure. Yield 0.86 g=86%

¹H-NMR (CDCl₃) 3.84 (s, 3H) 5.05 (s, 2H) 6.14 (s, 2H) 6.98 (m, 4H) 7.36 (d, 2H) 8.00 (d, 2H)

### e) (1S, 2S)-N-[cis-2-(6-fluoro-2-O-(4(4-methoxybenzyloxy)-benzoyloxymethyl)-3-propionylphenyl (cyclopropyl]-N′-[2-(5-cyanopyridyl)urea.

To a solution of (1S, 2S)-N-[cis-2-(6-fluoro-2-hydroxy-3-propionylphenyl)cyclopropyl]-N′-[2-(5-cyanopyridyl)]urea (368 mg, 1 mmole) in 5 ml DMF was added a suspension of 60% sodium hydride in mineral oil (44 mg, 1.1 mmole) and the mixture was stirred for one hour at room temperature. A solution of iodomethyl-4-(4-methoxybenzyloxy) benzoate (0.84 g, 2.1 mmole) in 2 ml THF was added and the mixture was stirred overnight at room temperature. 50 ml ethyl acetate were added and the organic phase was washed four times with water, dried with sodium sulfate and evaporated under reduced pressure. The product was isolated by silica gel column chromatography. Yield: 525 mg=82%

¹H-NMR (CDCl₃) 0.91 (m, 3H) 1.32 (m, 1H) 1.60 (m, 1H) 2.04 (m, 1H) 2.90 (m,2H) 3.20 (m, 1H) 3.82 (s, 3H) 5.04 (s, 2H) 5.84-6.06 (m, 2H) 6.91-8.18 (m, 1 3H)

### f) (1S, 2S)-N-[cis-2-(6-fluoro-2-O(4-hydroxybenzoyloxymethyl)-3-propionylphenyl)cyclopropyl]-N′-[2-(5-cyanopyridyl)]urea-O-methylene-4-hydroxybenzoate

To a solution of the intermediate of step e) (100 mg, 0.156 mmole) in 4 ml dichloromethane was added TFA (0.5 ml) and the solution was stirred for one hour at room temperature. The solution was evaporated under reduced pressure and the product was isolated by silica gel column chromatography. Yield: 45 mg=55%.

¹H-NMR (DMSO δ-6) 0.84 (m, 3H) 1.10 (m, 1H) 1.48 (m, 1H) 2.12 (m, 1H) 2.80 (m, 2H) 3.19 (m, 1H) 5.85-6.02 (m, 2H)6.84 (m, 2H) 7.18 (m, 1H) 7.46 (m, 2H) 7.74 (m, 2H) 8.04 (m, 2H) 8.38 (m, 1H)

### g) (1S, 2S)-N-[cis-2-(6-fluoro-2-O (4-isoleucyloxybenzoyloxymethyl)-3-propionylphenyl)-cyclopropyl]-N′-[2-(5-cyanopyridyl)]urea-O-methylene4-hydroxybenzoate-O-L-isoleucyl ester

An R₂ group, such as N-protected L-isoleucine is acylated to the exposed hydroxy group using conventional acylation conditions as described herein and deprotected to yield a compound of the invention.

### Biological Example 1

Bioavailability

The release of a compound of Formula P-2 from orally administered prodrugs of Formulae P3 to P8 were monitored in rats. The compounds of Examples 1 to 6 were made up in a propylene glycol vehicle and orally administered to paired fasted male Sprague Dawley rats at a dose corresponding to 0.027 mmol/kg. At 30, 60, 120, 240 & 360 minutes, 0.2 ml blood were collected, centrifuged and frozen for later analysis. The released drug of Formula P-2, (1S, 2S )-N-[cis-2-(6-fluoro-2-hydroxy-3-propionylphenyl)cyclopropyl]-N′-[2-(5-cyanopyridyl)]urea was assayed by HPLC. Aliquots comprising 40-100 μl of each plasma sample are mixed with an equal volume of acetonitrile (10 seconds, Vibrofex). The sample is centrifuged (2 min, 14000 RPM) and 30 μl the supernatant is injected into an HPLC system, as follows.

| | |
|---|---|
| Pre column: | RP-18, 7 μm, 15 × 3.2 mm |
| Column: | YMC basic, 3 μm, 150 × 3 mm |
| Mobile phase: | 60% acetonitrile in 3 mM ammonium acetate, pH 6.4 |
| Flow rate: | 0.4 ml/min |
| Detection: | UV, 250 nm |

TABLE P-1

| Example | Bioavailability_{0-6 hours} | Cₘₐₓ μM |
|---|---|---|
| 1 | 34% | 0.78 |
| 2 | 18% | 0.51 |
| 3 | 27% | 0.64 |
| 4 | 18% | 0.43 |
| 6 | 50% | 1.06 |
| 7 | 70% | 1.5 |

The above bioavailabilities correspond to sustained plasma levels of the active metabolite well above the ED₅₀ for HIV-1. The HIV-1 activity of the mother compound of formula P2 is described and quantitated in PCT/SE99/00194.

Although the invention has been illustrated by reference to examples employing particular mother NNRTIs, particular amino acids and particular linking groups it will be appreciated that the invention is not limited to these values but extends throughout the spirit and scope of the following claims.

1. A compound of the formula P1: 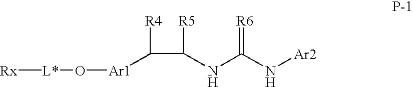
wherein:
Ar1 is an unsaturated, optionally substituted, mono or bicyclic ring structure comprising 0 to 3 hetero atoms selected from S, O and N;
Ar2 is an aromatic, optionally substituted, monocyclic ring structure comprising at least one nitrogen hetero atom and zero to two further hetero atoms selected from S, O and N;
R4 and R5 are independently H or C₃-C₈ cycloalkyl, C₁-C₆ alkyl, C₂-C₆ alkenyl, C₂-C₈ alkynyl, C₁-C₅ alkoxy, C₁-C₄ alkanoyloxy, C₁-C₄ alkylthio, amino, carboxy, carbamoyl, cyano, halo, hydroxy, aminomethyl, hydroxymethyl, carboxymethyl, or halo substituted C₁-C₆ alkyl mercapto, nitro;
or R4 and R5 join to form a 3-6 membered, optionally substituted ring structure;
R6 is O or S;
L* is a linker moiety which is ether-, carbonate- or ester-bound to the adjacent oxygen and ester linked to Rx;
Rx is the residue of a natural or unnatural amino acid;
and pharmaceutically acceptable salts thereof.

2. A compound according to claim 1, wherein L* comprises a C₁-C₈ alkyloxy group with an ether linkage to said hydroxy residue.

3. A compound according to claim 2, wherein the alkyloxy group has the formula: 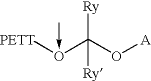
wherein the arrowed oxygen together with PETT is the hydroxy residue of formula I, Ry and Ry′ are independently H or C₁-C₃ alkyl and A is the Rx residue or an ester or carbonate bonded intermediate linking moiety to which Rx is esterified.

4. A compound according to claim 3, wherein Ry and Ry′ are both hydrogen.

5. A compound according to claim 2 wherein Rx is esterified to the alkyloxy group.

6. A compound according to claim 2 wherein L* further comprises an intermediate linking moiety esterified or carbonate bonded to the alkyloxy group, wherein the intermediate linking moiety possesses an hydroxy group to which Rx is esterified.

7. A compound according to claim 1, wherein L* comprises a carbonyl linkage to said hydroxy residue.

8. A compound according to claim 7, wherein L*′ further comprises an intermediate linking moiety possessing an hydroxy group to which Rx is esterified.

9. A compound according to claim 8 wherein the intermediate linking moiety is an optionally branched, optionally monounsaturated alkylenoxy group having 1-3 chain carbons.

10. A compound according to claim 9 wherein L*-Rx has the configuration: 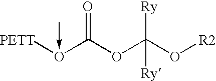
wherein Ry and Ry′ are independently H or C₁-C₃ alkyl and PETT together with the arrowed oxygen represents the hydroxy residue of the compound of claim 1.

11. A compound according to claim 10, wherein Ry and Ry′ are both hydrogen.

12. A compound according to claim 1, wherein Rx is derived from an aliphatic amino acid selected from alanine, leucine, isoleucine, tertiary leucine and valine.

13. A compound according to claim 12 wherein Rx is a derivative of an L-amino acid.

14. A compound according to claim 13 wherein Rx is a derivative of L-valine or L-isoleucine.

15. A compound according to claim 1, with the formula P2 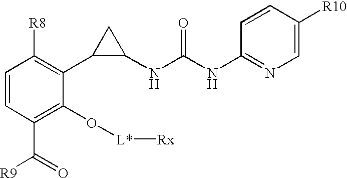
wherein
R8 is halo;
R9 is C₁-C₃ alkyl;
R10 is halo or cyano.

16. A compound according to claim 15, wherein R8 is fluoro.

17. A compound according to claim 15, wherein R9 is ethyl.

18. A compound according to claim 15, wherein R10 is cyano.

19. A compound according to claim 15, comprising at least 90% enantiomerically pure 1S 2S cis cyclopropyl configuration.

20. A compound according to claim 15, with the formula P3 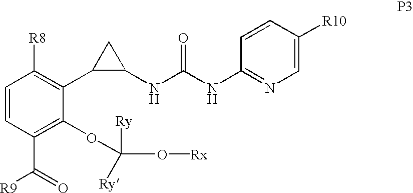
wherein
Ry and Ry′ are independently H or C₁₋₃ alkyl.

21. A compound according to claim 20, denoted:
(1S, 2S)-N-[cis-2-(6-fluoro, 2-(L-valyloxymethyloxy), 3-propionylphenyl)-cyclopropyl]-N′-(5-cyanopyrid-2-yl)-urea or (1S, 2S)-N-[cis-2-(6-fluoro, 2-(L-isoleucyloxymethyloxy), 3-propionylphenyl)-cyclopropyl]-N′-(5-bromopyrid-2-yl)-urea.

22. A compound according to claim 15 with the formula: 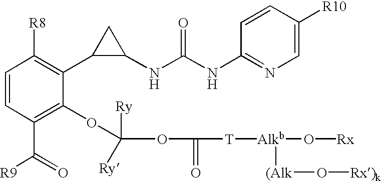
wherein
Alk^{b} is an optionally branched, optionally unsaturated C₁₋₆ alkyl,
Alk is C₁₋₆ alkyl,
T is a bond or —O—
Rx′ is the ester residue of a natural or unnatural amino acid, which may be the same or different to Rx;
and k is 0 or 1.

23. A compound according to claim 22, wherein Ry and Ry′ are each H.

24. A compound according to claim 22, wherein R₂ is derived from L-valyl or L-isoleucyl.

25. A compound according to claim 22, wherein T is —O—.

26. A compound according to claim 22, wherein T is a bond.

27. A compound according to claim 22, wherein Alk^{b} is 1,1 dimethylethylene, 2,2-dimethylethylene, propylene or butylene.

28. A compound according to claim 22 wherein k is 0.

29. A compound according to claim 22 denoted
(1S,2S)-N-[cis-2-(6-fluoro-2-(1,3-bis-L-valyloxy-2-(oxycarbonylmethoxy)propyl)-3-propionylphenyl)cyclopropyl]-N′-[2-(5-cyanopyridyl)]urea,
(1S,2S)-N-[cis-2-(6-fluoro-2-(1,3-bis-L-isoleucyloxy-2-(oxycarbonylmethoxy)propyl)-3-propionylphenyl)cyclopropyl]-N′-[2-(5-cyanopyridyl)]urea,
(1S,2S)-N-[cis-2-(6-fluoro-2-(1,3-bis-L-valyloxy-2-(oxycarbonylmethoxy)propyl)-3-propionylphenyl)cyclopropyl]-N′-[2-(5-bromopyridyl)]urea,
(1S,2S)-N-[cis-2-(6-fluoro-2-(1,3-bis-L-isoleucyloxy-2-(oxycarbonylmethoxy)propyl)-3-propionylphenyl)cyclopropyl]-N′-[2-(5-bromopyridyl)]urea,
(1S, 2S)-N-{cis-2-[6-fluoro-2-(2-(L-valyloxy)-ethoxycarbonyloxymethyloxy)-3-propionylphenyl)]cyclopropyl}-N′-[2-(5-cyanopyridyl)]urea,
(1S, 2S)-N-{cis-2-[6-fluoro-2-(2-(L-isoleucyloxy)-ethoxycarbonyloxymethyloxy)-3-propionylphenyl)]cyclopropyl}-N′-[2-(5-cyanopyridyl)]urea,
(1S, 2S)-N-{cis-2-[6-fluoro-2-(2-(L-valyloxy)-ethoxycarbonyloxymethyloxy)-3-propionylphenyl)]cyclopropyl}-N′-[2-(5-bromopyridyl)]urea,
(1S, 2S)-N-{cis-2-[6-fluoro-2-(2-(L-isoleucyloxy)-ethoxycarbonyloxymethyloxy)-3-propionylphenyl)]cyclopropyl}-N′-[2-(5-bromocyanopyridyl)]urea,

30. A compound according to claim 10 with the formula 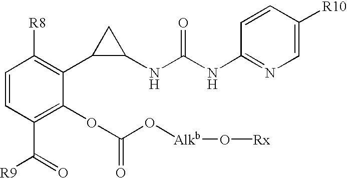
wherein Alk^{b} is optionally branched, optionally monounsaturated C₁-C₆ alkyl.

31. A compound according to claim 30, wherein Alk^{b} is methylene or, ethylene.

32. A compound according to claim 30 denoted
(1S,2S)-N-[cis-2-(6-fluoro-2-(L-valyloxymethoxycarbonyloxy)-3-propionylphenyl)cyclopropyl]-N′-[2-(5-cyanopyridyl)]urea,
(1S,2S)-N-[cis-2-(6-fluoro-2-(L-isoleucyloxymethoxycarbonyloxy)-3-propionylphenyl)cyclopropyl]-N′-[2-(5-cyanopyridyl)]urea,
(1S,2S)-N-[cis-2-(6-fluoro-2-(L-valyloxymethoxycarbonyloxy)-3-propionylphenyl)cyclopropyl]-N′-[2-(5-bromopyridyl)]urea,
(1S,2S)-N-[cis-2-(6-fluoro-2-(L-isoleucyloxymethoxycarbonyloxy)-3-propionylphenyl)cyclopropyl]-N′-[2-(5-bromopyridyl)]urea,
(1S,2S)-N-[cis-2-(6-fluoro-2-(2-(L-valyloxy)ethoxycarbonyloxy)-3-propionylphenyl)cyclopropyl]-N′-[2-(5-cyanopyridyl)]urea,
(1S,2S)-N-[cis-2-(6-fluoro-2-(2-(L-isoleucyloxy)ethoxycarbonyloxy)-3-propionylphenyl)cyclopropyl]-N′-[2-(5-cyanopyridyl)]urea,
(1S,2S)-N-[cis-2-(6-fluoro-2-(2-(L-valyloxy)ethoxycarbonyloxy)-3-propionylphenyl)cyclopropyl]-N′-[2-(5-bromopyridyl)]urea,
(1S,2S)-N-[cis-2-(6-fluoro-2-(2-L-isoleucyloxy)ethoxycarbonyloxy)-3-propionylphenyl)cyclopropyl]-N′-[2-(5-bromopyridyl)]urea,
(1S,2S)-N-[cis-2-(6-fluoro-2-(3-(L-valyloxy)propoxycarbonyloxy)-3-propionylphenyl)cyclopropyl]-N′-[2-(5-cyanopyridyl)]urea,
(1S,2S)-N-[cis-2-(6-fluoro-2-(3-(L-isoleucyloxy)propoxycarbonyloxy)-3-propionylphenyl)cyclopropyl]-N′-[2-(5-cyanopyridyl)]urea,
(1S,2S)-N-[cis-2-(6-fluoro-2-(3-(L-valyloxy)propoxycarbonyloxy)-3-propionylphenyl)cyclopropyl]-N′-[2-(5-bromopyridyl)]urea,
(1S,2S)-N-[cis-2-(6-fluoro-2-(3-(L-isoleucyloxy)propoxycarbonyloxy)-3-propionylphenyl)cyclopropyl]-N′-[2-(5-bromopyridyl)]urea,
(1S,2S)-N-[cis-2-(6-fluoro-2-(4-(L-valyloxy)butoxycarbonyloxy)-3-propionylphenyl)cyclopropyl]-N′-[2-(5-cyanopyridyl)]urea,
(1S,2S)-N-[cis-2-(6-fluoro-2-(4-(L-isoleucyloxy)butoxycarbonyloxy)-3-propionylphenyl)cyclopropyl]-N′-[2-(5-cyanopyridyl)]urea,
(1S,2S)-N-[cis-2-(6-fluoro-2-(4-(L-valyloxy)butoxycarbonyloxy)-3-propionylphenyl)cyclopropyl]-N′-[2-(5-bromopyridyl)]urea,
(1S,2S)-N-[cis-2-(6-fluoro-2-(4-(L-isoleucyloxy)butoxycarbonyloxy)-3-propionylphenyl)cyclopropyl]-N′-[2-(5-bromopyridyl)]urea,
or a pharmaceutically acceptable salt thereof.

33. A compound according to claim 32 denoted (1S,2S)-N-[cis-2-(6-fluoro-2-(L-valyloxymethoxycarbonyloxy)-3-propionylphenyl)cyclopropyl]-N′-[2-(5-cyanopyridyl)]urea or a pharmaceutically acceptable salt thereof.

34. A pharmaceutical composition comprising a compound as claimed in claim 1 and a pharmaceutically acceptable carrier therefor.

35. A pharmaceutical composition according to claim 34, further comprising one to three additional antivirals selected from AZT, ddI, ddC, D4T, 3TC, DAPD, abacavir, adefovir, adefovir dipivoxil, bis-POC-PMPA, emtricitabine, tamociclovir (H2G), valtamociclovir stearate (MIV-606), hydroxyurea, Hoechst-Bayer HBY 097, foscarnet (PFA), efavirenz, trovirdine, nevirapine, delaviridine, emivirine, DMP-450, loviride, ritonavir, ABT 378, saquinavir, indinavir, amprenavir (Vertex VX 478) and nelfinavir.

36. A compound as claimed in claim 1, for use in therapy.

37. Use of a compound as claimed in claim 1 in the preparation of a medicament for the treatment or prophylaxis of HIV infection.

38. A method for the treatment or prophylaxis of HIV in a subject in need thereof comprising the administration of an effective amount of a compound as defined in claim 1 to the subject.
